(19) United States
(12) Patent Application Publication
(10) Pub. No.: US 2026/0224670 A1
(43) Pub. Date: Aug. 6, 2026

(54) TREATMENT FOR CDKL5 DEFICIENCY DISORDER

(71) Applicant: The Francis Crick Institute Limited, London (GB)

(72) Inventors: Sila Ultanir, London (GB); Margaux Silvestre, London (GB)

(21) Appl. No.: 19/155,064

(22) PCT Filed: Feb. 9, 2024
(86) PCT No.: PCT/EP2024/053332

§ 371 Date: Aug. 8, 2025

(30) Foreign Application Priority Data
Feb. 10, 2023 (GB) 2301948.2
Jun. 12, 2023 (GB) 2308771.1

(51) Int. Cl. A61K 38/45; A61K 31/7088; A61K 45/06; A61K 48/00

(52) U.S. Cl. CPC A61K 38/45; A61K 31/7088; A61K 45/06; A61K 48/0033; A61K 48/0058

(57) ABSTRACT

Disclosed herein are methods and agents for the treatment and/or prevention of cyclin dependent kinase like 5 (CD-KL5) deficiency disorder. In particular, disclosed herein are compounds or compositions for increasing cyclin dependent kinase like 2 (CDKL2) in a subject, such as in the brain of a subject, and the use of those compounds and compositions in methods of treating CDKL5 deficiency disorder

The present invention relates to the treatment and/or prevention of CDKL5 deficiency disorder with compounds or compositions that increase cyclin dependent kinase like 2 (CDKL2) expression or activity.

### BACKGROUND

Brain development is an incredibly complex and intricate process, and any disturbance within it can have disastrous consequences. When such an issue arises, the resulting condition is labelled a neurodevelopment disorder (NDD), and can lead to an inability to reach cognitive, emotional and motor developmental milestones. As a result, patients can be permanently impaired in their motor functions, learning, communication, and more [1]. To date, they have been shown to affect more than 3% of children in the world, with few efficient treatments being available [2].

Clearly, those disorders are a real concern in our society, and developing a better understanding of them should be a priority. Finding therapies for those diseases has however been challenging, due to various reasons such as delayed diagnosis, complexity and heterogeneity of the diseases or lack of animal models. Despite those challenges, some successful treatments have been developed thanks to the persistent efforts of researchers and clinicians. One of the most significant accomplishments in recent years has been the gene therapy Zolgensma® that became a life-changing treatment for spinal muscular atrophy, a rare and often fatal genetic disease affecting the survival of motor neurones [3]. Other treatments for spinal muscular atrophy include an antisense oligonucleotide, nusinersen (Spinraza™), and a small molecule therapy, risdiplam (Evrysdi)

It is the hope that other diseases from that group can be met with similar progress. One of those conditions that is yet to be successfully tackled is CDKL5 deficiency disorder (CDD), which is characterised by early onset seizures and severe neurodevelopmental impairments, and caused by a mutation in a serine/threonine kinase called Cyclin-dependent kinase-like 5 (CDKL5).

As opposed to more common NDDs such as Autism spectrum disorder (ASD), which was first described in 1911, CDD was discovered relatively recently [4]. It was only in 2003 that a CDKL5 mutation observed in two girls was connected to infantile spasms and intellectual disabilities [5]. Before that, CDD was classified either as an early-onset seizure variant or Hanefeld variant of the Rett syndrome [6].

Despite increasing attention for CDKL5 and the growing evidence that CDKL5 mutations were linked to the symptoms, it was only in 2013 that CDD was considered as a distinct clinical entity. This only occurred because the clinical features of CDD and Rett syndrome patients were compared and a distinct clinical profile for CDD was found [7].

In 2018, the first phase-3 clinical trial including CDD patients began, when they tested the effect of an antiepileptic compound named ganaxolone. In 2022, ganaxolone became the only FDA-approved treatment indicated specifically for CDD. CDD was finally granted a World Health Organisation classification in 2020 that made the disorder more visible for clinical research and helped patient carers to have access to insurance.

The prevalence of CDD is now estimated at approximatively 1 in 40,000 to 60,000 births, but due to how recent CDD still is, new cases are still being identified at a rapid pace [8, 9, 10]. CDD patients are predominantly females as male patients represent only 1:4 of the affected population. Moreover, male patients tend to be more severely impacted, suggesting that CDKL5 mutation is mostly lethal in males during the foetus development [11, 12, 10].

NDDs are often caused by a combination of various factors, both environmental (i.e. infections, malnutrition, social deprivation or physical trauma) and genetic. However, CDD is one of the rare NDDs that is exclusively caused by a genetic mutation. CDD is the result of de novo mutations in the CDKL5 gene, which is located on the short arm of the X chromosome (Xp22). One familial case of CDKL5 mutation was reported in two siblings, which was likely due to maternal germline mosaicism [13].

More than 265 pathogenic mutations on CDKL5 were reported, with half of them being point mutations. Thirty eight percent of those point mutations are missense mutations, which represents the most common mutations in CDD patients [14]. Moreover, these missense mutations are all (except in one case) located on the catalytic domain of CDKL5, and patients show more severe symptoms. This increased severity demonstrates the importance of the CDKL5 activity in the disease [15, 16, 10]. In addition, 13% of CDD patients have frameshift mutations due to point insertion or deletion [17,12]. Several cases of partial or complete deletion of CDKL5 were also reported and even an instance of partial deletion of the short arm of the X chromosome has been identified [18, 19, 20, 21].

Duplication of CDKL5 was also reported in several patients and all of them show autistic features, developmental delays, language impairments and hyperactivity but were missing epilepsy, which is the key clinical manifestation of CDD [22]. Those cases suggest that CDKL5 might be dosage sensitive and misregulation of CDKL5 expression has significant consequences on brain development. Finally, two genetically identical twin siblings were diagnosed with CDD in 2015 with the same mutation but their phenotype was very different [23], which could be explained by X chromosome inactivation. However, this last example could also be explained by environmental or epigenetic factors, opening the possibility for a broader explanation for CDD, which would go beyond a purely genetic cause.

### SUMMARY OF THE INVENTION

The present invention provides a compound or composition for increasing cyclin dependent kinase like 2 (CDKL2) expression or activity.

In particular, compounds or compositions as described herein are suitable for increasing CDKL2 expression or activity in the brain.

In some embodiments, the compound or composition comprises a nucleic acid molecule, for example a small interfering RNA (siRNA), a guide RNA (gRNA), or a short hairpin RNA (shRNA). In some embodiments, the compound or composition comprises a nucleic acid molecule encoding a CDKL2 polypeptide. In some embodiments, the nucleic acid molecule encoding CDKL2 encodes a polypeptide having at least 80%, at least 85%, at least 90%, at least 95% or at least 99% identity to a polypeptide of SEQ ID NO:1. In some embodiments, the nucleic acid molecule encoding CDKL2 encodes a polypeptide comprising the sequence of SEQ ID NO:1. In some embodiments, the nucleic acid molecule encoding CDKL2 encodes a polypeptide consisting of the sequence of SEQ ID NO: 1.

In some embodiments, the nucleic acid molecule encoding CDKL2 comprises a nucleotide sequence having at least 80%, at least 85%, at least 90%, at least 95% or at least 99% identity to a nucleotide sequence selected from the list consisting of SEQ ID NO:2 and SEQ ID NO:3. In some embodiments, the nucleic acid molecule encoding CDKL2 comprises a nucleotide sequence selected from the list consisting of SEQ ID NO:2 and SEQ ID NO:3. In some embodiments, the nucleic acid molecule encoding CDKL2 consists of a nucleotide sequence selected from the list consisting of SEQ ID NO: 2 and SEQ ID NO:3.

In some embodiments, the compound or composition comprises a viral vector. In some embodiments, the viral vector comprises one or more of an adenoviral vector, an adeno-associated viral vector, a lentiviral vector or a retroviral vector. In some embodiments, the nucleic acid molecule further comprises a promoter.

In some embodiments, the compound or composition comprises one or more of Clustered Regularly Interspaced Short Palindromic Repeat (CRISPR) associated nucleases, Transcription activator-like effector nucleases (TALEN) or Zinc finger nucleases (ZNF). In some embodiments, the compound or composition comprises a CRISPR-associated protein (Cas protein) and a guide RNA (gRNA) that hybridizes within a CDKL2 genomic sequence.

In some embodiments, the CDKL2 genomic sequence is defined by Ensembl reference ENSG00000138769 (SEQ ID NO:8).

In some embodiments, the compound or composition comprises an isolated CDKL2 polypeptide. In some embodiments, the compound or composition comprises a polypeptide having at least 80%, at least 85%, at least 90%, at least 95% or at least 99% to a polypeptide of SEQ ID NO: 1. In some embodiments, the compound or composition comprises a polypeptide of SEQ ID NO:1. In some embodiments, the compound or composition consists of a polypeptide of SEQ ID NO:1.

In some embodiments, the compound or composition comprises or further comprises a small molecule activator of CDKL2 expression or activity. In some embodiments, the compound or composition comprises a small molecule activator that specifically binds to the protein structure defined in PDB database reference 4BBM. In some embodiments, the compound or composition comprises or further comprises a polypeptide that increases CDKL2 expression. In some embodiments, the compound or composition comprises a transcription factor or an enhancer that increases CDKL2 expression. In some embodiments, the compound or composition comprises or further comprises an antibody. In some embodiments, the compound or composition comprises a fusion protein comprising a CDKL2 polypeptide operatively linked to a second polypeptide sequence. In some embodiments, the second polypeptide sequence comprises a nuclear localization signal (NLS), a nuclear export signal (NES), a leader signal polypeptide or a cell-penetrating polypeptide.

In some embodiments, the compound or composition increases CDKL2 activity by at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450% or at least 500% relative to the CDKL2 activity measured in the absence of the compound or composition. In some embodiments, CDKL2 activity is measured by the level of phosphorylation of a CDKL2 substrate and/or the level of CDKL2 autophosphorylation.

In some embodiments, CDKL2 activity is measured by the level of phosphorylation of microtubule-associated protein RP/EB family member 2 (MAPRE2/EB2), microtubule-associated protein 1S (MAP1S), or voltage-dependent R-type calcium channel subunit alpha-1E (CACNA1E). In some embodiments, the microtubule-associated protein RP/EB family member 2 (MAPRE2/EB2) comprises or consists of the amino acid sequence of SEQ ID NO:5, the microtubule-associated protein 1S (MAP1S) comprises or consists of the amino acid sequence of SEQ ID NO:6, or the voltage-dependent R-type calcium channel subunit alpha-1E (CACNA1E) comprises or consists of the amino acid sequence of SEQ ID NO:7.

In some embodiments, CDKL2 activity is measured by the level of phosphorylation of microtubule-associated protein RP/EB family member 2 (MAPRE2/EB2), optionally wherein the microtubule-associated protein RP/EB family member 2 (MAPRE2/EB2) comprises or consists of the amino acid sequence of SEQ ID NO:5.

In some embodiments, the compound or composition increases CDKL2 expression by at least 10%, at least 20%, at least 30%, at least 40%, at least 50%, at least 60%, at least 70%, at least 80%, at least 90% or at least 100%, at least 150%, at least 200%, at least 250%, at least 300%, at least 350%, at least 400%, at least 450% or at least 500% relative to the CDKL2 expression measured in the absence of the compound or composition. In some embodiments, CDKL2 expression is measured by western blot, quantitative real-time PCR (qRT-PCR), advanced RNA sequencing, microarray, mass spectrometry, ELISA or Phos-tag gel electrophoresis.

In some embodiments, the compound or composition further comprises a pharmaceutically acceptable carrier or excipient. In some embodiments, the compound or composition further comprises a second therapeutic agent useful for the treatment of CDKL5 deficiency disorder. In some embodiments, the second therapeutic agent is selected from the list consisting of: clobazam, topiramate, prednisolone/prednisone, adrenocorticotropic hormone and ganaxolone.

The present invention also provides a method of increasing cyclin dependent kinase like 2 (CDKL2) expression or activity in a subject in need thereof comprising administering a compound or composition according to the invention to the subject. The present invention also provides a method of treating or preventing a disease or disorder in a subject, the method comprising administering a compound or composition according to the invention to the subject. The present invention also provides a method of treating or preventing a neuronal disease or disorder in a subject, the method comprising administering a compound or composition according to the invention to the subject. The present invention also provides a method of treating or preventing a cyclin dependent kinase like 5 (CDKL5) deficiency disorder in a subject, the method comprising administering a compound or composition according to the invention to the subject.

The present invention also provides a compound or composition according to the invention for use in a method of increasing cyclin dependent kinase like 2 (CDKL2) expression or activity in a subject in need thereof. The present invention also provides a compound or composition as described herein for use in a method of increasing cyclin dependent kinase like 2 (CDKL2) expression or activity in the brain of a subject. The present invention also provides a compound or composition according to the invention for use in a method of treating or preventing a disease or disorder in a subject. The present invention also provides a compound or composition according to the invention for use in a method of treating or preventing a neuronal disease or disorder in a subject. The present invention also provides a compound or composition according to the invention for use in a method of treating or preventing a cyclin dependent kinase like 5 (CDKL5) deficiency disorder in a subject. The present invention also provides a compound or composition as described herein for use in a method of treating or preventing a cyclin dependent kinase like 5 (CDKL5) deficiency disorder in a subject, wherein the compound or composition is capable of increasing cyclin dependent kinase like 2 (CDKL2) expression or activity in the brain of the subject.

The present invention also provides a use of a compound or composition according to the invention for increasing cyclin dependent kinase like 2 (CDKL2) expression or activity in a subject in need thereof. The present invention also provides a use of a compound or composition according to the invention for treating or preventing a disease or disorder in a subject. The present invention also provides a use of a compound or composition according to the invention for treating or preventing a neuronal disease or disorder in a subject. The present invention also provides a use of a compound or composition according to the invention for treating or preventing a cyclin dependent kinase like 5 (CDKL5) deficiency disorder in a subject.

In some embodiments, the compound or composition is administered intrathecally, intravenously, intracistnerally, intracerebroventrically or intraparenchymally. In some embodiments, the subject is a mammal. In some embodiments, the subject is a human. In some embodiments, the compound or composition increases cyclin dependent kinase like 2 (CDKL2) expression or activity in the subject.

The application also provides a compound or composition according to the invention for increasing cyclin dependent kinase like 2 (CDKL2) expression or activity in a subject.

The application also provides a composition comprising a cyclin dependent kinase like 2 (CDKL2) activator. In some embodiments, the composition further comprises a pharmaceutically acceptable carrier or excipient.

### BRIEF DESCRIPTION OF THE DRAWINGS

Embodiments of the present invention will now be described, by way of example only, with reference to the accompanying drawings, in which:

FIG. 1—CDKL5 activity is present in excitatory and inhibitory neurons. (A) Schematic representation of different Cre lines used to delete CDKL5 in specific brain cell types. (B) Residual pS222 EB2 is observed in CDKL5 KO (−Y) male mice when compared to wild type littermates (+/Y), indicating that a different kinase or kinases can phosphorylate EB2 at Ser222 in addition to CDKL5.

FIG. 2—Some microtubule-associated protein RP/EB family member 2 (MAPRE2/EB2) phosphorylation remains in CDKL5 KO cells. (A) and (B) show the reduction in CDKL5 levels (A) and pSer222 EB2 phosphorylation (B) when CDKL5 is knocked out in full body (KO), or cell type specific drivers. See FIG. 1A for colour coding of Cre drivers. The data shows that CDKL5 and its activity (pEB2) is present in excitatory (green) and inhibitory (yellow & orange) neurons. CDKL5 activity was not observed in astrocytes (purple).

FIG. 3—phospho-Ser222 is present at low levels in CDKL5 KO. Remaining pS222 EB2 levels in P8 cortex in full body CDKL5 knockout, indicating that another kinase/kinases can phosphorylate EB2. Lack of pS222 signal in EB2 phosphomutant mouse cortex confirms that no background is observed with the pS222 antibody used.

FIG. 4—Rat primary cortical neurons phospho-Ser222 is increased upon okadaic acid (OA) and reduced with N-methyl-d-aspartate (NMDA). These data show that in rat primary neurons, phosphatase inhibitor treatment (okadaic acid/OA) for 1 hour causes increase in pS222 EB2, indicating that this site is dephosphorylated by PP1/PP2A phosphatases under normal conditions.

FIG. 5—phospho-Ser222 can be increased with okadaic acid (OA) and reduced with N-methyl-d-aspartate (NMDA) in cultured neurons from CDKL5 KO mice. In primary cortical cultures made from CDKL5 KO embryos, OA treatment causes an increase in phosphor-S222, indicating that a kinase other than CDKL5 can phosphorylate this site. NMDA treatment reduces the pS222 signal indicating that NMDA downregulates this phosphorylation and that the signal is not background. (B) is quantification of the blots shown in (A).

FIG. 6—phospho-Ser222 can be increased with okadaic acid (OA) and reduced with N-methyl-d-aspartate (NMDA) in cultured neurons from CDKL5 KO mice. Primary cultures from male wild type littermate control embryos is shown compared to CDKL5 KO cultures. Side by side display of wild type and CDKL5 knockout littermate cultures from cortex, pS222 EB2 is much lower in CDKL5 KOs, yet, the remaining pS222 can still be upregulated by OA and downregulated by NMDA, indicating that some pS222 EB2 remains in CDKL5 KOs and can be regulated. (B) is quantification of the blots shown in (A). (C) shows that in each culture there is a consistent increase of pSer222 EB2 by OA.

FIG. 7—ICK and CDKL2 phosphorylate microtubule-associated protein RP/EB family member 2 (MAPRE2/EB2) in HEK293 cells. These data show a screen testing if other kinases can phosphorylate pS222 EB2. Kinases were expressed in HEK293 cells. Only CDKL5, ICK and CDKL2 can phosphorylate EB2. (A) shows a tree of kinases that could potentially have overlapping substrates with CDKL5. (B) shows the ability of different recombinantly expressed kinases to phosphorylate EB2. (C) shows the ratio of pS222 EB2: total EB2 for each of the kinases, indicating that significant phosphorylation is seen only with CDKL5, ICK and CDKL2.

FIG. 8—ICK and CDKL2 phosphorylate microtubule-associated protein RP/EB family member 2 (MAPRE2/EB2) in HEK293 cells. (A) Western blots and (B) quantification of expression of ICK, CDKL2 and CDKL5 wild type and kinase dead (KD) forms in HEK293 cells (ICK left panel, CDKL2 central panel, CDKL5 right panel). These data show that all three kinases can phosphorylate S222 on EB2.

FIG. 9—CDKL5 KO primary cortical neurons transfected with FLAG tagged CDKL2, ICK or CDKL5 show increased phospho-Ser222 in transfected neurons. These data show expression of kinases CDKL5, ICK and CDKL2 in primary cortical neurons. pS222 EB2 is specifically increased in the neurons that express these kinases indicating that CDKL2, ICK and CDKL5 can phosphorylate EB2 in neurons.

FIG. 10—CDKL5 KO primary neurons infected with CDKL2 shRNA have reduced phospho-microtubule-associated protein RP/EB family member 2 (MAPRE2/EB2) upon treatment with okadaic acid. Primary neurons from CDKL5 KO mouse cortices were used to knock down CDKL2 by lentiviral expression of shRNAs targeting CDKL2. Both shRNAs caused a reduction of the OA dependent increase of pEB2/EB2. Data indicates that CDKL2 is the other kinase responsible for EB2 phosphorylation in CDKL5 KO neurons.

FIG. 11—ICK expression pattern is different than CDKL5 whereas CDKL2 expression overlaps with CDKL5 expression. Using the public Allen Brain Institute database, single cell mRNA expression of the kinases of interest were analysed in adult human brain cells. ICK and CDKL4 expression is very low in human neurons. Therefore, CDKL2 is the most likely candidate for phosphorylating CDKL5 substrates, such as EB2, in neurons.

FIG. 12—Exemplary CDKL2 amino acid and RNA sequences. Disclosed in this figure are examplary amino acid and mRNA sequences encoding CDKL2. SEQ ID NO 1 represents an amino acid sequence a known isoform of CDKL2 (Uniprot reference: Q92772). SEQ ID NOs 2 and 3 represent exemplary mRNA sequences encoding CDKL2 (RefSeq references NM_003948 and NM_001330724).

FIG. 13—Sequence alignment of human CDKL and RCK kinases. The protein sequences of the eight kinases were aligned using ICM software. The red squares highlight amino acids that are common between CDKL5, ICK and CDKL2.

FIGS. 14—3D structure alignment of human CDKL and RCK kinases. A. Superimposition of the protein 3D structures of the kinase domain of CDKL5 (orange), ICK (purple) and CDKL2 (pink) using ICM software. Gatekeeper residue is shown as sticks. B. Alignment of the protein 2D structures of the kinase domain of human CDKL and RCK kinases using ICM software.

FIG. 15—Exemplary sequences of microtubule-associated protein RP/EB family member 2 (MAPRE2/EB2), derived from UniProt reference Q15555-1 (SEQ ID NO:5), microtubule_associated protein 1S (MAP1S), derived from UniProt reference Q66K74-1 (SEQ ID NO: 6) and voltage-dependent R type calcium channel subunit alpha-1E (CACNA1E), derived from UniProt reference Q15878-1 (SEQ ID NO: 7).

FIG. 16—A depiction of the cut sites in genomic DNA which excise Exon 4 of CDKL2, generating a CDKL2 knockout mouse. Exon 4 contains essential coding sequence for the ATP binding pocket of CDKL2, without it no functional kinase can be formed.

FIG. 17—Western blot analysis of knockout mouse brain lysates. All mice are CDKL5 knockouts (KO). CDKL2 is either wild type (wt), heterozygous knockout (+/−) or homozygous knockout (−/−). A. Two different ages have been analysed postnatal day 10 (P10) and P16. EB2 phospho Ser222 levels were used as a measure of CDKL5 signaling. pS222 EB2 is reduced in +/− mice when compared to wts and is further reduced in −/− at P10. At P16 WT and −/− were compared and there is a robust reduction in pS222 EB2 in −/− mice. B. Phospho Ser222 EB2/total EB2 was reduced in CDKL2 homozygous (hom) deletion when compared to wild type (wt) mice. CDKL2 deletion reduces the remaining EB2 phosphorylation in CDKL5 KOs to approximately 15% at P10. Upper and lower bands represent EB2 splice isoforms. Upper band was quantified for the graph in panel B.

FIG. 18—Spontaneous alternation in the Y-Maze is reduced in CDKL5/2 double knockout mice compared to wild-type mice. Male and female wild-type (n=3 males, n=3 females), CDKL5 knockout (n=3 males, n=4 females) and CDKL5/2 double knockout (n=3 males, n=3 females) had their performance in the Y-maze compared. A one-way ANOVA found a significant effect of genotype on Y-maze performance [F (2, 16)=5.661, p=0.0138], with a moderate effect size (R²) of 0.4144. Tukey's multiple comparisons test showed that, compared to wild-type mice, CDKL5/2 double knockout mice demonstrated a significantly reduced proportion of alternations [p=0.0119]. Significant (p<0.05) differences are indicated with *. Error bars are standard deviations.

### DETAILED DESCRIPTION OF THE INVENTION

In order for the present invention to be more readily understood, certain terms are first defined below. Additional definitions for the following terms and other terms are set forth throughout the specification.

As used herein, the terms “cyclin-dependent kinase-like 2”, “cyclin dependent kinase like 2” or “CDKL2” refer to an amino acid sequence encoded by the CDKL2 gene (HGNC ref 1782). The gene is also known as KKIAMRE or P56. The gene is encoded by the genomic sequence identified by Ensembl reference ENSG00000138769. Transcripts ENST00000307465.9 and ENST00000429927.6 can also be used to define the CDKL2 sequence. In some embodiments, CDKL2 may comprise an amino acid sequence of SEQ ID NO: 1 (Uniprot reference: Q92772). In some embodiments, CDKL2 may consist of an amino acid sequence of SEQ ID NO: 1 (Uniprot references: Q92772). In some embodiments, CDKL2 may be encoded by an mRNA sequence comprising a sequence selected from any one of SEQ ID NO:2 and SEQ ID NO:3 (RefSeq references NM_003948 and NM_001330724). In some embodiments, CDKL2 may be encoded by an mRNA sequence consisting of a sequence selected from any one of SEQ ID NO:2 and SEQ ID NO:3 (RefSeq references NM_003948 and NM_001330724). In some embodiments, the CDKL2 protein may have a structure as defined in PDB database reference 4BBM [24]. In a preferred embodiment CDKL2 comprises an amino acid sequence of SEQ ID NO: 1 (Uniprot reference: Q92772).

As used herein, the term “compound or composition for increasing cyclin dependent kinase like 2 (CDKL2) expression or activity” or “cyclin dependent kinase like 2 (CDKL2) activator” refers to any agent that increases expression or activity of cyclin dependent kinase like 2 (CDKL2) polypeptide or protein. In particular, the compounds or compositions aim to increase CDKL2 expression or activity in the brain. The increase in expression or activity can be measured by any standard means known in the art, including measuring an increase in the level of CDKL2-encoding polynucleotide (such as CDKL2 mRNA) and/or CDKL2 polypeptide or protein. Methods for quantifying the amount of one or more CDKL2 polynucleotides include fluorescent in situ hybridization (FISH), spectroscopy, quantitative sequencing, quantitative PCR (qPCR), RT-PCR, ddPCR, Southern blotting or northern blotting. Methods for quantifying the amount of one or more CDKL2 polypeptides or protein include immunohistochemistry (IHC), spectroscopy, colorimetric detection, Western blotting, mass spectrometry or ELISA. The level of CDKL2 activation or increased activity provided by an agent that increases expression or activity of CDKL2 may be at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450% or at least 500% of the expression or activity of CDKL2 when measured in the absence of the agent that increases expression or activity of CDKL2. Ideally, levels of CDKL2 activity or expression are compared in the same model system (e.g. cell line or tissue) in the presence and in the absence of the agent that increases expression or activity of CDKL2.

In some embodiments, an increase in expression or activity of cyclin dependent kinase like 2 (CDKL2) polypeptide or protein may be measured by an increase in the level of phosphorylation of a CDKL2 substrate. In some embodiments, the CDKL2 substrate is microtubule-associated protein RP/EB family member 2 (MAPRE2/EB2), microtubule-associated protein 1S (MAP1S) or voltage-dependent R-type calcium channel subunit alpha-1E (CACNA1E). In some embodiments, the CDKL2 substrate is microtubule-associated protein RP/EB family member 2 (MAPRE2/EB2). In some embodiments, the sequence of microtubule-associated protein RP/EB family member 2 (MAPRE2/EB2) is the sequence set out in UniProt reference Q15555-1 (disclosed herein as SEQ ID NO: 5). In some embodiments, the sequence of microtubule-associated protein 1S (MAP1S) is the sequence set out in UniProt reference Q66K74-1 (disclosed herein as SEQ ID NO: 6). In some embodiments, the sequence of voltage-dependent R-type calcium channel subunit alpha-1E (CACNA1E) is the sequence set out in UniProt reference Q15878-1 (disclosed herein as SEQ ID NO: 7). The level of increased CDKL2 substrate phosphorylation provided by an agent that increases expression or activity of CDKL2 may be at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450% or at least 500% of the CDKL2 substrate phosphorylation when measured in the absence of the agent that increases expression or activity of CDKL2. Ideally, levels of CDKL2 substrate phosphorylation is compared in the same model system (e.g. cell line or tissue) in the presence and in the absence of the agent that increases expression or activity of CDKL2. In some embodiments, the level of microtubule-associated protein RP/EB family member 2 (MAPRE2/EB2) phosphorylation in the presence of an agent that increases expression or activity of CDKL2 may be at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450% or at least 500% of the CDKL2 substrate phosphorylation when measured in the absence of the agent that increases expression or activity of CDKL2. Ideally, levels of CDKL2 substrate phosphorylation is compared in the same model system (e.g. cell line or tissue) in the presence and in the absence of the agent that increases expression or activity of CDKL2.

As used herein, the term “CDKL5 deficiency disorder” or “CDD” refers to a clinical manifestation of phenotypes associated with a deficiency in CDKL5 expression or activity. In some embodiments, the manifestation of CDD may be defined by parameters set out in the prior art, for example in [10]. In some embodiments, the manifestation of CDD may be defined by parameters set out in the prior art, for example those defined by the Online Mendelian Inheritance in Man (OMIM) database reference 300672 [25]. In some embodiments, the manifestation of CDD may be defined by parameters set out in the prior art, for example for example those defined by the International Classification of Diseases (ICD) database (10^{th} edition) reference G40.42 [26].

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%), or less in either direction (greater than or less than) of the stated reference value unless otherwise stated or otherwise evident from the context (except where such number would exceed 100% of a possible value).

As used herein, the term “prevention” means delaying or forestalling the onset, development or progression of a disease, disorder, or condition for a period of time from minutes to indefinitely. “Prevention” also includes reducing the risk of developing a disease, disorder, or condition. “Prevention” includes, but does not require, complete avoidance of a disease condition.

As used herein, the term “treatment” (also “treat” or “treating”) refers to any administration of a therapeutic agent according to a therapeutic regimen that achieves a desired effect in that it partially or completely alleviates, ameliorates, relieves, reduces severity of and/or reduces incidence of one or more symptoms or features of a particular disease, disorder, and/or condition. In some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. In a preferred embodiment of the invention the disease, disorder, and/or condition is CDKL5 deficiency disorder.

The term “comparable”, as used herein, refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

The term “correlates”, as used herein, has its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

As used herein, the terms “improve,” “increase” or “reduce,” or grammatical equivalents, indicate values that are relative to a reference (e.g., baseline) measurement, such as a measurement taken under comparable conditions (e.g., in the same individual prior to initiation of treatment described herein, or a measurement in a control individual (or multiple control individuals) in the absence of treatment) described herein.

As used herein, a “polypeptide” is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides may optionally include “non-natural” amino acids or other entities that nonetheless are capable of integrating into a polypeptide chain.

As used herein, the term “protein” refers to a molecule comprising a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

As used herein, the term “subject”, “individual”, or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.). In a preferred embodiment of the invention the subject is a human.

As used herein, the terms “target cell” or “target tissue” refers to any cell, tissue, or organism. In preferred embodiments, the target cell or target tissue is a liver cell such as a hepatocyte or liver tissue.

As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include administration of one or more doses, optionally spaced apart by regular or varied time intervals. In some embodiments, a therapeutic regimen is one whose performance is designed to achieve and/or is correlated with achievement of (e.g., across a relevant population of cells, tissues, or organisms) a particular effect, e.g., reduction or elimination of a detrimental condition or disease. In some embodiments, treatment includes administration of one or more therapeutic agents either simultaneously, sequentially or at different times, for the same or different amounts of time. In some embodiments, a “treatment regimen” includes genetic methods such as gene therapy, gene ablation or other methods known to induce or reduce expression (e.g. transcription, processing, and/or translation of a particular gene product, such as a primary transcript or mRNA).

As used herein, the term “therapeutically effective amount” refers to an amount of a therapeutic agent which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of or feels an effect). In some embodiments, “therapeutically effective amount” refers to an amount of a therapeutic agent or composition effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts.

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “transcription” or “transcribed” refers to the first of several steps of DNA based gene expression in which a target sequence of DNA is copied into RNA (especially mRNA) by the enzyme RNA polymerase. During transcription, a DNA sequence is read by an RNA polymerase, which produces a complementary, antiparallel RNA sequence called a primary transcript.

As used herein, “target sequence” means a sequence to which a compound is intended to hybridize to result in a desired activity with respect to CDKL2 expression. Oligonucleotides have sufficient complementarity to their target sequences to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In both DNA and RNA, guanine (G) is complementary to cytosine (C). In some embodiments, complementary nucleobase means a nucleobase of an oligomeric compound that is capable of base pairing with a nucleobase of its target sequence. For example, if a nucleobase at a certain position of an oligomeric compound is capable of hydrogen bonding with a nucleobase at a certain position of a target sequence, then the position of hydrogen bonding between the oligomeric compound and the target sequence is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another. As used herein, “complementary” in reference to oligomeric compounds (e.g., linked nucleosides, oligonucleotides) means the capacity of such oligomeric compounds or regions thereof to hybridize to a target sequence, or to a region of the oligomeric compound itself, through nucleobase complementarity.

As used herein, “fully complementary” in reference to a nucleic acid sequence, amino acid sequence or region thereof means that each nucleobase of the oligomeric compound or region thereof is capable of pairing with a nucleobase of a complementary nucleic acid target sequence or a self-complementary region of the oligomeric compound. Thus, a fully complementary oligomeric compound or region thereof comprises no mismatches or unhybridized nucleobases with respect to its target sequence or a self-complementary region of the oligomeric compound.

As used herein, “percent complementarity” means the percentage of nucleobases or amino acids of a nucleic acid sequence or amino acid sequence that are complementary to an equal-length portion of a target nucleic acid sequence or amino acid sequence. Percent complementarity is calculated by dividing the number of nucleobases or amino acids of the nucleic acid sequence or amino acid sequence that are complementary to nucleobases or amino acids at corresponding positions in the target nucleic acid sequence or amino acid sequence by the total length of the nucleic acid sequence or amino acid sequence.

As used herein, “percent identity” means the number of nucleobases or amino acids in a first nucleic acid sequence or amino acid sequence that are the same type (independent of chemical modification) as nucleobases or amino acids at corresponding positions in a second nucleic acid sequence or amino acid sequence, divided by the total number of nucleobases or amino acids in the first nucleic acid sequence or amino acid sequence.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression.

As used herein, “region” or “regions”, or “portion” or “portions”, mean a plurality of linked nucleobases or amino acids that have a function or character as defined herein, in particular with reference to the claims and definitions as provided herein. Typically, such regions or portions comprise at least 10, at least 11, at least 12 or at least 13 linked nucleobases or amino acids. For example, such regions can comprise 13 to 20 linked nucleobases or amino acids, such as 13 to 16 or 18 to 20 linked nucleobases or amino acids.

As used herein, “pharmaceutically acceptable carrier or diluent” means any substance suitable for use in administering to an animal. In some embodiments, a pharmaceutically acceptable carrier or diluent is sterile saline. In some embodiments, such sterile saline is pharmaceutical grade saline.

As used herein, “amino” includes primary, secondary and tertiary amino groups. As used herein, “halo” and “halogen,” mean an atom selected from fluorine, chlorine, bromine and iodine.

The term “comprising” is used herein to mean including the method steps or elements identified, but that such steps or elements do not comprise an exclusive list and as such there may be present additional steps or elements.

Further, to the extent that the term “includes” is used in either the detailed description or the claims, such term is intended to be inclusive in a manner similar to the term “comprising” as “comprising” is interpreted when employed as a transitional word in a claim.

### Clinical Definitions of CDD

Key clinical features of CDD were defined in 2019 to help with diagnosis. CDD is defined by a pathogenic or likely pathogenic variant in the CDKL5 gene, motor and cognitive development delays and epilepsy with onset in the first year of life [10].

Epilepsy is the most prominent clinical feature of CDD, with 90% of the patients showing epileptic episodes by the age of three months. Those episodes occur daily in 8% of cases, and weekly in 20% of cases. Notably, 43.6% of CDD patients exhibit a honeymoon period lasting between 2.5 months and 6 years during which epilepsy stops. CDD patients undergo three stages of epilepsy during their life: (1) Infantile spasms, (2) epileptic encephalopathy, and (3) refractory epilepsy. Infantile spasm is the most common stage of epilepsy in CDD patients, as it is the first stage in 23% of patients, and occurs at some point in the life of 81% of patients.

Another key clinical manifestation of CDD is hypotonia which is now almost considered as a universal feature. CDD patients also commonly show cortical visual impairment in 75% of the cases, with reports of poor eye contact and lack of visual tracking. In addition, CDD patients show severe developmental delays and intellectual disabilities, as they tend to reach key milestones at a slower pace or never reach them at all. For example, only 49% of the female patients can walk independently by the age of five, and only 7.5% of all female patients can form full sentences.

In addition to key features, part of the affected population also exhibits accompanying symptoms, which are thought to be indirect consequences of the disease. The most notable ones are autistic features, hand stereotypies, gastrointestinal symptoms, orthopaedic complications respiratory and cardiac abnormalities or sleep disorder. Gastrointestinal symptoms such as constipation, reflux and air swallowing are the most common accompanying symptoms, occurring in 86.5% of individuals. Hand stereotypies is another common symptom that affect 80% of CDD patients. 68.5% of the patients have scoliosis by the age of ten, due to the hypotonia and rehabilitation, orthopaedic corsets or surgical treatment are often necessary. Carers have also reported abnormal sleep duration and an increase of night waking in 63% of CDD patients. Finally, a substantial portion of CDD patients were reported to suffer breathing abnormalities and cardiac arrhythmia.

Some variability was reported in the severity of the symptoms in CDD patients, as is the case for independent walking, where 23% of patients are able to do so but 77% cannot. Another example of that is the fact that 33.5% of patients can communicate using words or body language, whereas the remaining patients are not able to express themselves. This variability could be explained by the localisation or the type of mutation on CDKL5 but the correlation seems to be limited. It seems however that patients with mutations in, or upstream of, the catalytic domain demonstrate an increased severity of their symptoms [15,27]. In addition, 8.8% of CDD patients were reported to have somatic mosaicism [28], and the ratio of mosaicism could explain the observed variability.

However, the possibility of a link between mosaicism and the variability of symptoms remains to be established. Inactivation of the X chromosome in female patients could be another reason for the variability. Approximately half of their cells express a non-functional CDKL5 and, depending on the cell types that express the mutant allele, the severity of symptoms could vary. Similar to mosaicism, no correlation was found yet.

### Pharmaceutical Compositions of the Agent

As used herein “pharmaceutical composition” means a mixture of substances suitable for administering to an individual. For example, a pharmaceutical composition may comprise one or more active pharmaceutical agents and a sterile aqueous solution.

As used herein “pharmaceutically acceptable salts” means physiologically and pharmaceutically acceptable salts of compounds, i.e., salts that retain the desired biological activity of the parent compound and do not impart undesired toxicological effects thereto.

Other aspects of the present invention also relate to a medicinal product or a diagnostic aid comprising a compound or composition for increasing cyclin dependent kinase like 2 (CDKL2) expression or activity according to the invention or a cell according to the invention and, where appropriate, suitable excipients and additives, such as, for example, a physiological saline solution, stabilizers or proteinase inhibitors.

### Kits

Any pharmaceutical composition described herein can be provided in a kit. In some instances, the kit includes (a) a container that contains a pharmaceutical composition described herein and, optionally (b) informational material. The informational material can be descriptive, instructional, marketing or other material that relates to the methods described herein and/or the use of an agent, e.g., for therapeutic benefit.

The informational material of the kits is not limited in its form. In some instances, the informational material can include information about production of a compound or composition for increasing cyclin dependent kinase like 2 (CDKL2) expression or activity according to the invention, concentration, date of expiration, batch or production site information, and so forth. In other situations, the informational material relates to methods of administering a compound or composition for increasing cyclin dependent kinase like 2 (CDKL2) expression or activity according to the invention, e.g., in a suitable amount, manner, or mode of administration (e.g., a dose, dosage form, or mode of administration described herein).

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about a therapeutic agent therein and/or their use in the methods described herein. The informational material can also be provided in any combination of formats.

In addition to a pharmaceutical composition of the invention, the kit can include other ingredients, such as a solvent or buffer, a stabilizer, or a preservative. The kit can also include further agents, e.g., a second or third agent, e.g., other therapeutic agents or other therapeutic compounds or compositions. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for a pharmaceutical composition or other agents. In some cases, the kit contains separate containers, dividers or compartments for a therapeutic agent and informational material. For example, a therapeutic agent can be contained in a bottle, vial, or syringe, and the informational material can be contained in a plastic sleeve or packet. In other situations, the separate elements of the kit are contained within a single, undivided container. For example a compound or composition for increasing cyclin dependent kinase like 2 (CDKL2) expression or activity according to the invention can be contained in a bottle, vial or syringe that has attached thereto the informational material in the form of a label. In some cases, the kit can include a plurality (e.g., a pack) of individual containers, each containing one or more unit dosage forms (e.g., a dosage form described herein) of a compound or composition for increasing cyclin dependent kinase like 2 (CDKL2) expression or activity according to the invention. The containers can include a unit dosage, e.g., a unit that includes a therapeutic agent. For example, the kit can include a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit can optionally include a device suitable for administration of a compound or composition for increasing cyclin dependent kinase like 2 (CDKL2) expression or activity according to the invention, e.g., a syringe or other suitable delivery device. The device can be provided preloaded with a compound or composition for increasing cyclin dependent kinase like 2 (CDKL2) expression or activity according to the invention, e.g., in a unit dose, or can be empty, but suitable for loading.

### Vectors

The present invention also provides vectors comprising any one or more of the nucleic acid molecules disclosed herein, such as vectors encoding a CDKL2 DNA or mRNA coding sequence. In some embodiments, the invention provides a nucleic acid sequence encoding the amino acid sequence of SEQ ID NO:1. Vectors according to the invention may comprise a nucleic acid sequence encoding the amino acid sequence of SEQ ID NO:1. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, lentiviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Vectors as described herein may comprise adeno-associated virus (AAV) particles that are capable of crossing the blood-brain barrier. Such AAVs may also be capable of transducing cells of the central nervous system (CNS), such as neurons. AAVs for this purpose are known in the art, and may comprise mutated or variant capsid proteins that confer the ability to cross the blood-brain barrier, The compound or composition that increases CDKL2 as described herein may be delivered as part of a payload in combination with the AAVs, particularly when the compound or composition is a nucleic acid molecule. Accordingly, described herein is a recombinant adeno-associated virus (AAV) particle comprising an AAV capsid protein and a payload, wherein the payload comprises a compound or composition for increasing CDKL2 expression or activity, and wherein the recombinant AAV is capable of crossing the blood-brain barrier.

Described herein is a recombinant adeno-associated virus (AAV) particle comprising an AAV capsid protein and a nucleic acid payload, wherein the nucleic acid payload encodes a polypeptide having at least 80%, at least 85%, at least 90%, at least 95% or at least 99% identity to a polypeptide of SEQ ID NO:1, and wherein the recombinant AAV is capable of crossing the blood-brain barrier.

Described herein is a recombinant adeno-associated virus (AAV) particle comprising an AAV capsid protein and a nucleic acid payload, wherein the nucleic acid payload encodes a polypeptide of SEQ ID NO: 1, and wherein the recombinant AAV is capable of crossing the blood-brain barrier.

Described herein is a recombinant adeno-associated virus (AAV) particle comprising an AAV capsid protein and a nucleic acid payload, wherein the nucleic acid payload comprises a nucleotide sequence having at least 80%, at least 85%, at least 90%, at least 95% or at least 99% identity to a nucleotide sequence selected from the list consisting of SEQ ID NO:2 and SEQ ID NO:3, and wherein the recombinant AAV is capable of crossing the blood-brain barrier.

Described herein is a recombinant adeno-associated virus (AAV) particle comprising an AAV capsid protein and a nucleic acid payload, wherein the nucleic acid payload comprises a nucleotide sequence selected from the list consisting of SEQ ID NO:2 and SEQ ID NO:3, and wherein the recombinant AAV is capable of crossing the blood-brain barrier.

### CRISPR

As used herein, “CRISPR nuclease system” refers collectively to transcripts and other elements involved in the expression of or directing the activity of CRISPR-associated (“Cas”) genes, including sequences encoding a Cas gene, a guide sequence (also referred to as a “spacer” in the context of an endogenous CRISPR system), or other sequences and transcripts from a CRISPR locus.

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. A CRISPR system is characterized by elements that promote the formation of a CRISPR complex at the site of a target sequence.

In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. in some embodiments, the target sequence may be within an organelle of a eukaryotic cell, for example, mitochondrion or chloroplast.

When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell. In some embodiments, a vector comprises a regulatory element operably linked to an enzyme-coding sequence encoding a CRISPR enzyme, such as a Cas protein, also called Cas enzyme.

Non-limiting examples of Cas proteins (or Cas enzymes) include Cas1, Cas1.13, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm.5, Csm6, Cmr1, Cmr3, Cmr4, Cmr5, Crnr6, Csb1, Csb2, Csb3, Csx17, Csx14, Csx10, Csx16, CsaX, Csx3, Csx1, Csx15, Csf1, Csf2, Csf3, Csf4, hornologs thereof, or modified versions thereof.

These enzymes are known; for example, the amino acid sequence of S. pyogenes Cas9 protein may be found in the SwissProt database under accession number Q99ZW2. In some embodiments, the CRISPR enzyme has DNA cleavage activity, such as Cas9. In some embodiments the CRISPR enzyme is Cas9, and may be Cas9 from S. pyogenes or S. pneumoniae.

In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

### Alternative Modalities

In some embodiments, the compound or composition for increasing cyclin dependent kinase like 2 (CDKL2) expression or activity comprises a small molecule. In some embodiments, the compound or composition for increasing cyclin dependent kinase like 2 (CDKL2) expression or activity comprises an activating peptide. In some embodiments, the compound or composition for increasing cyclin dependent kinase like 2 (CDKL2) expression or activity comprises an antibody.

### Combination Therapies

In some embodiments, the invention features a composition (e.g., one or more compositions, formulations or dosage formulations) or a pharmaceutical combination, comprising a compound or composition for increasing cyclin dependent kinase like 2 (CDKL2) expression or activity according to the invention and a second therapeutic agent. In some embodiments, the second therapeutic agent is chosen from one or more agents selected from the list consisting of clobazam, topiramate, prednisolone/prednisone, adrenocorticotropic hormone and ganaxolone.

In some embodiments, the composition comprises a pharmaceutically acceptable carrier. In some embodiments, the compound or composition for increasing cyclin dependent kinase like 2 (CDKL2) expression or activity according to the invention and the second agent can be present in a single composition or as two or more different compositions. The compound or composition for increasing cyclin dependent kinase like 2 (CDKL2) expression or activity according to the invention and the second agent can be administered via the same administration route or via different administration routes. The compound or composition for increasing cyclin dependent kinase like 2 (CDKL2) expression or activity according to the invention and the second agent can be administered simultaneously or sequentially. In some embodiments, the pharmaceutical combination comprises the compound or composition for increasing cyclin dependent kinase like 2 (CDKL2) expression or activity according to the invention and the second agent separately or together.

### Routes of Administration

The compound or composition for increasing cyclin dependent kinase like 2 (CDKL2) expression or activity according to the invention or pharmaceutical composition according to the invention can be administered by different routes including orally, parenterally, sublingually, transdermally, rectally, transmucosally, topically, via inhalation, via buccal administration, intrapleurally, intravenously, intraarterially, intraperitoneally, subcutaneously, intramuscularly, intranasal, intrathecally, and/or intraarticularly, intracerebroventricular injection, or combinations thereof. In some embodiments the agent or pharmaceutical composition is administered orally. In some embodiments the agent or pharmaceutical composition is administered intravenously. In some embodiments the agent or pharmaceutical composition is administered by intracerebroventricular injection. In some embodiments the compound or composition for increasing cyclin dependent kinase like 2 (CDKL2) expression or activity according to the invention or pharmaceutical composition is administered subcutaneously. Preferably, the compound or composition is administered in a manner that facilitates delivery to the brain of a subject. Preferably, the compound or composition is administered such that CDKL2 expression or activity is increased in the brain of a subject.

As described herein, the compounds and compositions of the invention preferably increase CDKL2 expression or activity in the brain of a subject. In order to facilitate such an increase, the compounds or compositions may be formulated or delivered in a way that facilitates delivery to the brain. For example, the compounds or compositions described herein may be formulated or delivered such that they cross the blood-brain barrier. The compounds or compositions described herein may be formulated or delivered such that they target cells of the CNS. One suitable approach utilises modified AAV capsid proteins which cross the blood-brain barrier and target CNS cells. For example, when the compound that increases CDKL2 is a nucleic acid molecule, the nucleic acid molecule may be delivered as a payload as part of a recombinant AAV particle. An alternative approach to achieve brain or CNS-specific delivery is to conjugate a compound or composition as described herein to a targeting agent, such as an agent that targets the brain. The targeting agent may be an agent that crosses the blood brain barrier. Such agents (or conjugates) allow targeted drug delivery to the brain, and are described in (for example) EP2308514. Such agents (or conjugates) may be particularly useful where the compound or composition that increases CDKL2 is a peptide, a protein, or a small molecule. The compounds or compositions described herein may be conjugated to the targeting ligand via a linker.

### EXAMPLES

### Example 1

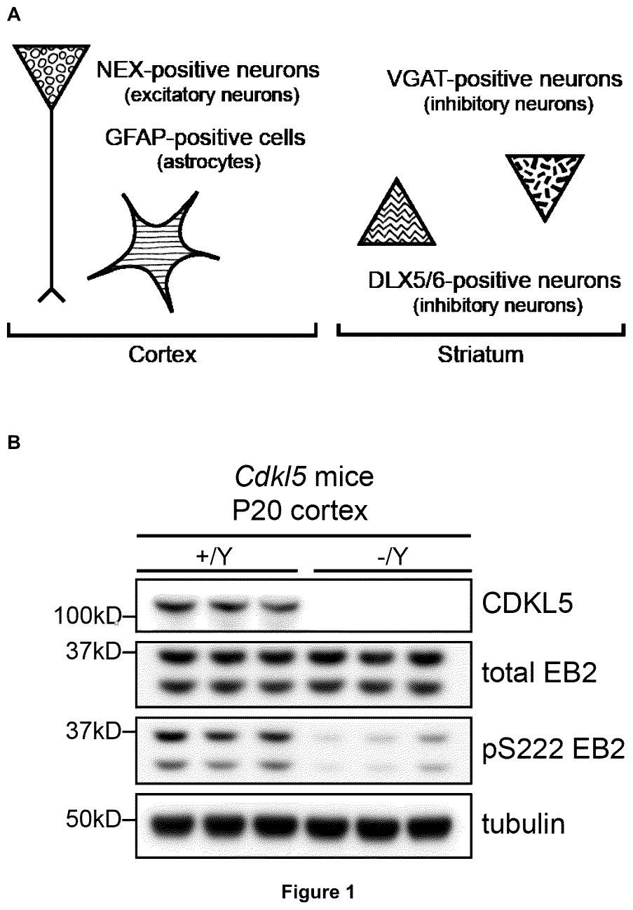

FIGS. 1-10 relate to Rabbit anti-microtubule-associated protein RP/EB family member 2 (MAPRE2/EB2) phospho-specific pS222 antibody which was generated using the same method as described in document [29].

### Example 2

FIGS. 1, 2 and 3 relate to animal experiments. The mice were bred and handled according to the regulation of the animal (Scientific Procedures) Act 1986 of the United Kingdom, and approved by the Francis Crick Institute ethical committee. The mice were housed and maintained on a 12 hours light/dark cycle and provided with food and water ad libidum. Each mouse strain was backcrossed into C57BL/6J genetic background.

Cdkl5 full KO mice were a generous gift from Cornelius Gross at the European Molecular Biology Laboratory in Rome [30]. Heterozygous females and wild-type males were used to maintain Cdkl5 full KO mouse line and only the brains of male offspring were harvested to ensure Cdkl5 WT and hemizygous KO littermates.

Residual pS222 EB2 is observed in CDKL5 KO (−Y) male mice when compared to wild type littermates (+/Y), indicating that a different kinase or kinases can phosphorylate EB2 at Ser222 in addition to CDKL5 (FIG. 1).

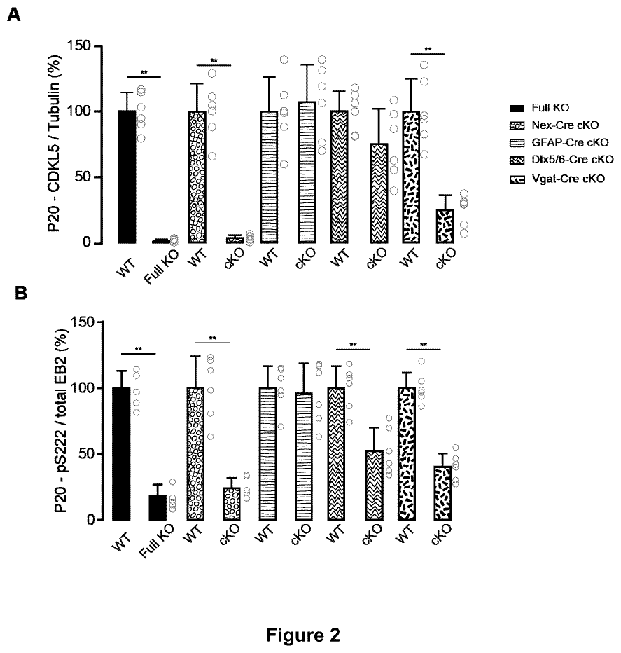

There is a reduction in CDKL5 levels (A) and pSer222 EB2 phosphorylation (B) when CDKL5 is knocked out in full body (KO), or cell type specific drivers. See FIG. 1A for colour coding of Cre drivers. The data shows that CDKL5 and its activity (pEB2) is present in excitatory (green) and inhibitory (yellow & orange) neurons. CDKL5 activity was not observed in astrocytes (purple) (FIG. 2).

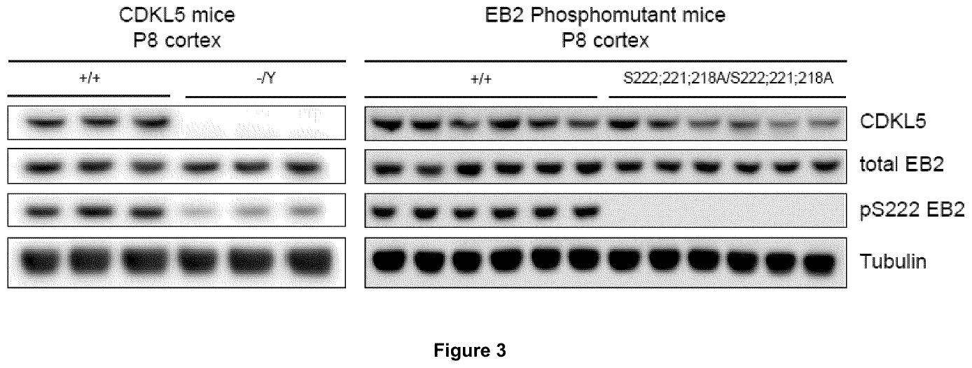

Remaining pS222 EB2 levels in P8 cortex in full body CDKL5 knockout, indicating that another kinase/kinases can phosphorylate EB2. Lack of pS222 signal in EB2 phosphomutant mouse cortex confirms that no background is observed with the pS222 antibody used (FIG. 3).

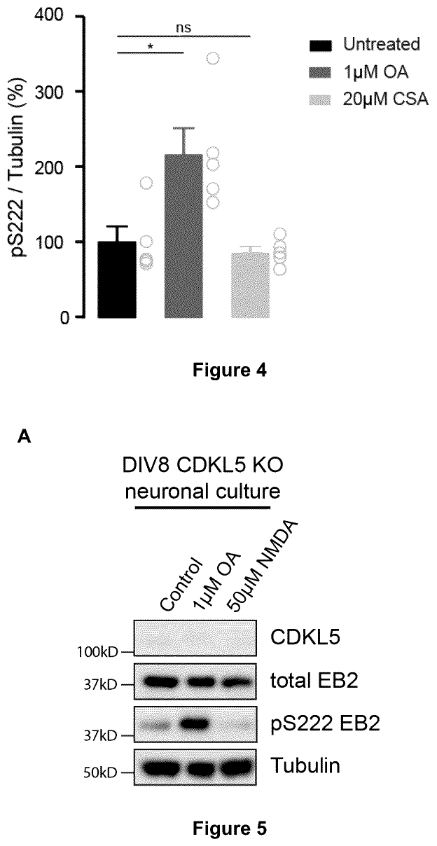

In rat primary neurons, phosphatase inhibitor treatment (okadaic acid/OA) for 1 hour causes increase in pS222 EB2, indicating that this site is dephosphorylated by PP1/PP2A phosphatases under normal conditions (FIG. 4).

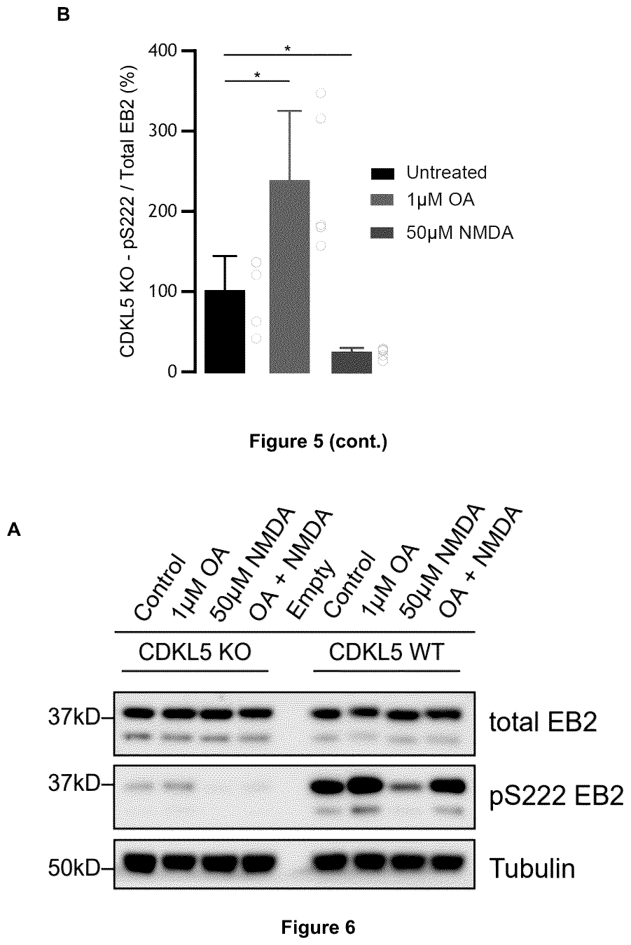

In primary cortical cultures made from CDKL5 KO embryos, OA treatment causes an increase in phosphor-S222, indicating that a kinase other than CDKL5 can phosphorylate this site. NMDA treatment reduces the pS222 signal indicating that NMDA downregulates this phosphorylation and that the signal is not background (FIG. 5).

### Example 3

In order to delete CDKL5 in specific cell types and generate Cdkl5 conditional KO (cKO) mouse models, homozygous Cdkl5 floxed females were crossed with Cdkl5 heterozygous males Nex-Cre for excitatory neurones, Dlx5/6-Cre or Vgat-Cre for inhibitory neurones and Gfap-Cre for astrocytes. Brains were harvested at P20 from Cdkl5 hemizygous floxed males expressing Cre and Cre-negative littermates as controls (FIG. 2). Cdkl5 floxed females were also a gift from Cornelius Gross [30]. Nex-Cre mouse model (Neurod6tm1(cre) Kan, MGI: 2668659) was a generous gift from Dr. Klaus Nave. Dlx5/6-Cre mouse model (Tg(ml56i-cre, EGFP)1Kc, MGI: 3609985) was a generous gift from Dr. Francois Guillermot at the Francis Crick Institute. Gfap-Cre mouse model (B6.Cg-Tg (Gfap-cre) 77.6 Mv; Stock Number: 012887) was purchased from Jackson Laboratories. Vgat-Cre (Slc32 atm2(cre)Lowl/J, Stock Number: 016962) was a generous gift from Dr. Andreas Schaefer at the Francis Crick Institute.

### Example 4

Microtubule-associated protein RP/EB family member 2 (MAPRE2/EB2) phosphomutant mice were generated by the Genetic Modification Service at the Francis Crick Institute using CRISPR/Cas9 system. C57BL/6J superovulated females were mated with C57BL/6J males and embryos were then microinjected with 25 ng/μL of guide RNA (gRNA), 100 ng of WT Cas9 mRNA and 100 ng/μL of repair template. gRNA was generated using in vitro transcription (IVT). In short, annealed gRNA primers were ligated to a cloning vector, then IVT was performed on the gRNA plasmid using the MEGAshortscript kit (Ambion) and gRNA was finally purified using the MEGAclear kit (Ambion).

Two pairs of gRNA primers were designed and tested (Table 1) and either set of gRNA sequences are suitable for generating the mutant mice. WT Cas9 mRNA was purchased from Tebubio. The repair template was ordered from Integrated DNA Technologies and had the following sequence:

(SEQ ID NO: 4)
5′-TTC TGC TTG CTT CTA GGC GCG GCT AAG TCA AGT CCT GCA TCT AAG CCA GGA TCC ACA
CCT GCT CGC CCC GCG GCT GCC AAA AGG GCT TCA TCC AGT GGC TCT GCA TCC AGA TCT
GAC AAA GAT TT A GAA ACG CAG GTC AT A CAG CTC AAC GAG CAG GTA ATT CAT CGC CTC
CAC CCT TGT-3′.

| Primers | Sequence (5′-3′) |
|---|---|
| Forward gRNA primer 1 | AGGGTGAAGCCCTTTTGGCAGACG (SEQ ID NO: 9) |
| Reverse gRNA primer 1 | AAACCGTCTGCCAAAAGGGCTTCA (SEQ ID NO: 10) |
| Forward gRNA primer 2 | AGGGAGACGAGGGGCGAGAAGGTG (SEQ ID NO: 11) |
| Reverse gRNA primer 2 | AAACCACCTTCTCGCCCCTCGTCT (SEQ ID NO: 12) |
| Forward genotyping primer | TCGTCGGCAGCGTCAGATGTGTATAAGAGACAG |
| | (SEQ ID NO: 13) |
| Reverse genotyping primer | GTCTCGTGGGCTCGGAGATGTGTATAAGAGACAG |
| | (SEQ ID NO: 14) |

### Table 1—Primers for the Generation of the Eb2 Phosphomutant Mouse Model

The genotype of the modified pups was checked by extracting genomic DNA from the ear clip and amplifying the gRNA target sites with PCR using the primers indicating in Table 2.1. The obtained PCR products were cleaned using AMPure XP beads (Beckman Coulter) and sequenced using MiSeq (Illumina).

### Example 5

Harvesting brains (FIGS. 1-3). For Western blot. Fresh brains were taken promptly from mice after cervical dislocation and decapitation. When desired, brain regions were dissected, and tissue was flash frozen in liquid nitrogen.

### Example 6—Cdkl5 WT and KO Primary Culture

Brains from individual male E16.5 embryos from heterozygous Cdkl5 mothers were collected and placed in hibernate buffer (Gibco) while the Cdkl5 wildtype (WT) and KO genotype was determined (FIGS. 4, 5, 6, 9, 10). An equal number of both genotypes was selected, and cortices and hippocampi from those embryos were then dissected and individually washed three times with Hank's balanced salt solution (HBSS). An incubation with 0.25% trypsin for 15 minutes at 37° C. was followed by four times washing with HBSS. Cells were dissociated and then counted using a haemocytometer. Neurones were plated on 18 mm glass coverslips (Fisher) or directly on the plastic well of 12-well culture plates at a density between 150,000 and 300,000 cells. The coverslips and the wells were coated with 0.1 M borate buffer containing 60 μg/mL poly-D-lysine and 2.5 μg/mL laminin and placed in the incubator overnight. Neurones were plated with minimum essential medium (MEM) containing 10% foetal bovine serum (FBS), 0.5% dextrose, 0.11 mg/mL sodium pyruvate, 2 mM glutamine and penicillin/streptomycin. After 4 hours, cultures were transferred to neurobasal medium containing 1 mL of B27 (Gibco), 0.5 mM GlutaMAX, 0.5 mM glutamine, 12.5 μM glutamate and penicillin/streptomycin. Primary neuronal cultures were kept at 37° C. and 5% CO2. Every 3-4 days, 20-30% of the maintenance media was refreshed.

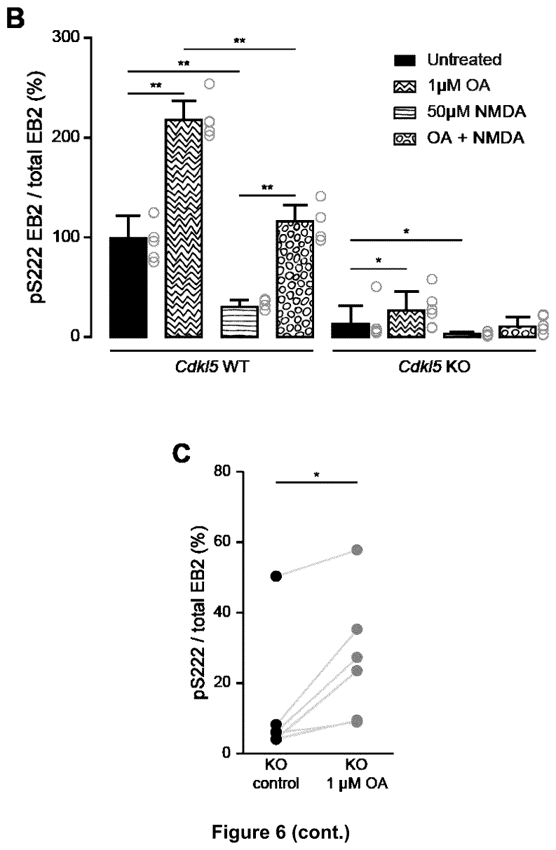

Primary cultures from male wild type littermate control embryos compared to CDKL5 KO cultures show that pS222 EB2 is much lower in CDKL5 KOs, yet, the remaining pS222 can still be upregulated by OA and downregulated by NMDA, indicating that some pS222 EB2 remains in CDKL5 KOs and can be regulated (FIG. 6).

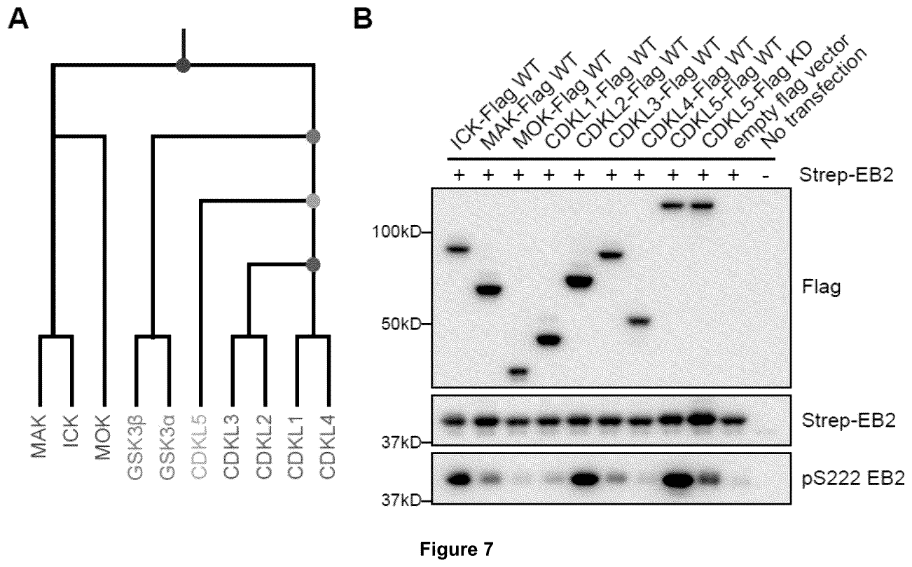

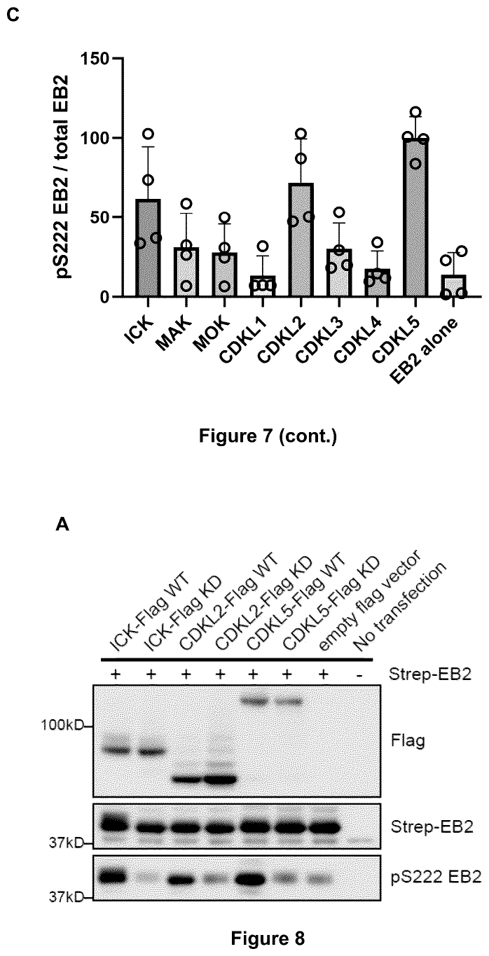

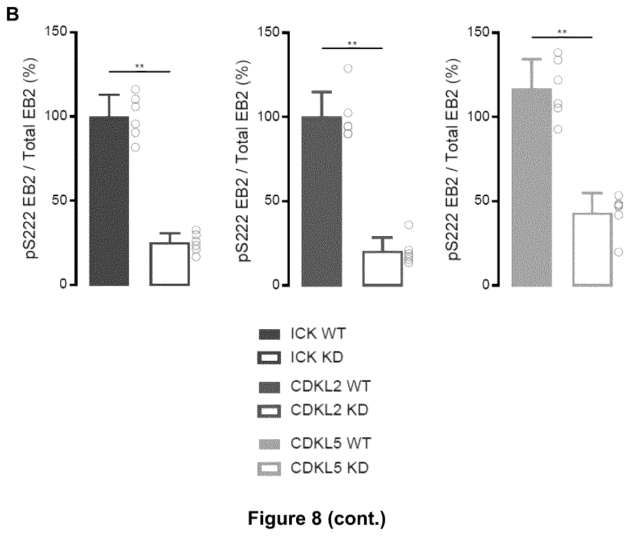

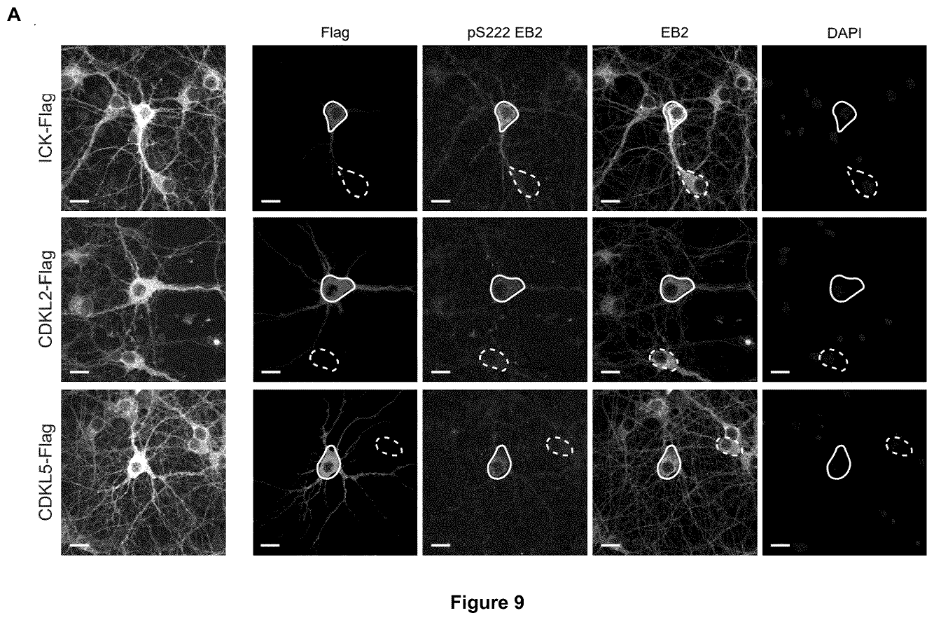

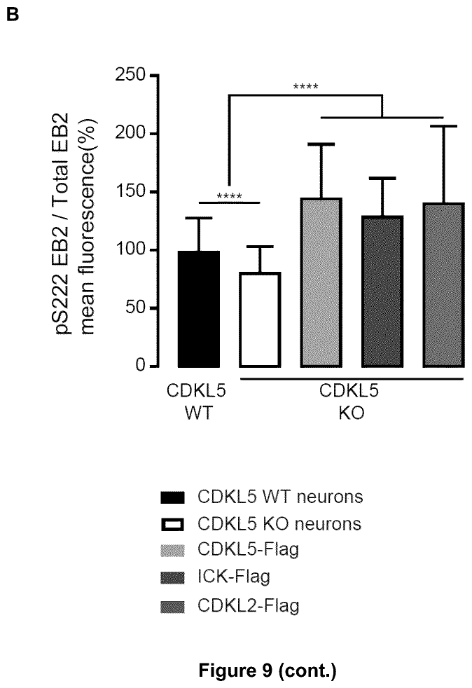

A screen for other related kinases that may phosphorylate pS222 EB2 showed that only CDKL5, ICK and CDKL2 can phosphorylate EB2 (FIG. 7). Further data showed that all three kinases can phosphorylate S222 on known CDKL5 substrate EB2 in HEK293 cells (FIG. 8) and primary cortical neurons (FIG. 9).

Pregnant Long Evans rats were ordered from Jackson. E18.5 rat embryos were removed from the uterus and the brains were taken out. Cortices were dissected out, pooled from multiple animals and washed three times with HBSS. The rest of the protocol is identical to the one used for the mouse primary neurones (see Example 9).

### Example 7—HEK293T Cells

HEK293T cells were grown in Dulbecco's modified Eagle medium (DMEM) supplemented with FBS and penicillin/streptomycin at 37° C. with 5% CO2 for maximum 25 passages (FIGS. 7-9). Each passage consisted in dissociating the cells using 0.25% trypsin-EDTA for 3 minutes at 37° C. and adding 1:10 of the cells into a new T75 flask. The remaining cells could be used to be plated for future experiments. This step was repeated every 3-4 days. For all the experiment, cells were plated onto 12-well plates and, 24 hours after passage, cells were transfected.

### Example 8

pLVExp-CMV-EGFP-rELAVI4 WT plasmid was purchased from VectorBuilder. rELAVI4 phosphomutant (SA) plasmid was generating by introducing the mutation S131A using site-directed mutagenesis (FIGS. 7-10). pRK5-HA-CDKL51-352 (kinase domain) WT and kinase dead (KD) vectors as well as pTriex6-StrepII-EB2 vector were already described in [29]. hCDKL1, hCDKL2, hCDKL3, hMAK and hMOK were purchased in a pDONR223 vector from Addgene (respectively #23389, #23421, #23628, 23624 and #23645) and were transferred into a pCAG-DEST-3×FLAG-TwinStrep vector using the Gateway cloning method (Invitrogen). hCDKL4 (NM_001346911.1) and hICK (NM_014920.4) were purchased in a pcDNA3.1+/C-(K)-DYK vector from Genscript and were transferred into a pCAG-DEST-3×FLAG-TwinStrep vector using the Gateway cloning method (Invitrogen). In order to generate kinase dead plasmids, K33 34 36R for ICK, K33R for CDKL2 and K42R D153A for CDKL5 mutations were generated using site-directed mutagenesis. The final plasmids were respectively called ICK-FLAG KD, CDKL2-FLAG KD and CDKL5-FLAG KD.

### Example 9—Neuronal Culture Treatment

Cortical primary rat neurones or Cdkl5 cortical primary neurones were plated onto 12-well plates and treated by adding directly the treatment to the media (FIGS. 4-6). The neurones were then incubated at 37° C. before being lysed in 300 μL of 1× sample buffer (Invitrogen) containing 0.1 M DTT. The final concentration and the length of the treatment depend of the assay but are indicated in the result sections. The zero-time point is a control condition where water or DMSO was added to the well for the longest time point of the assay. For the NMDA treatment in absence of calcium, the medium of the neurones was changed to artificial cerebrospinal fluid (ACSF) buffer (125 mM NaCl, 2.5 mM KCl, 26 mM NaHCO₃, 1.25 mM NaH2CO3, 25 mM glucose, 2 mM CaCl2) and 1 mM MgCl2) or to Ca2+-free ACSF buffer (125 mM NaCl, 2.5 mM KCl, 26 mM NaHCO₃, 1.25 mM NaH2CO3, 25 mM glucose, 10 mM EGTA and 1 mM MgCl2). The neurones were then treated and lysed the same way. For the inhibition of synaptic NMDA receptors, the neurones were pre-incubated for 10 minutes with 10 UM MK-801 (Tocris) and 50 UM bicuculline (Tocris), then washed with fresh new media, treated with NMDA and finally lysed.

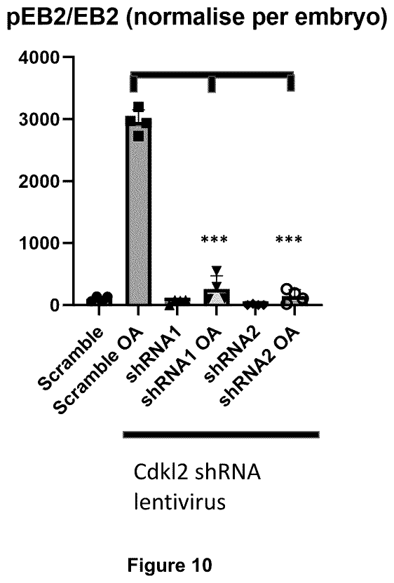

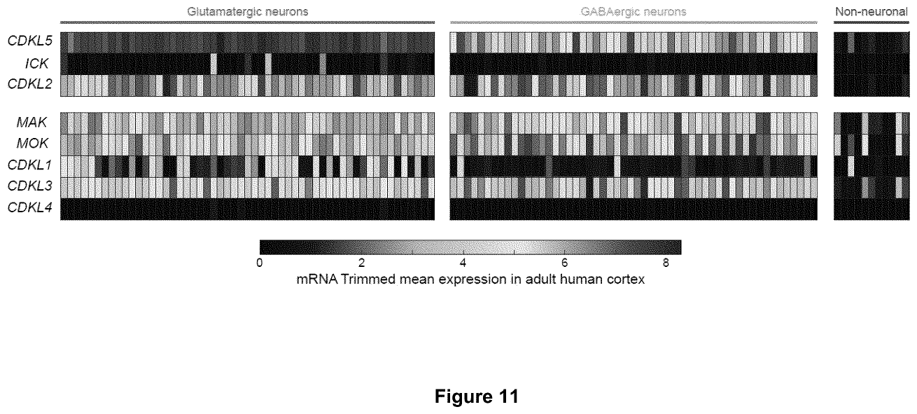

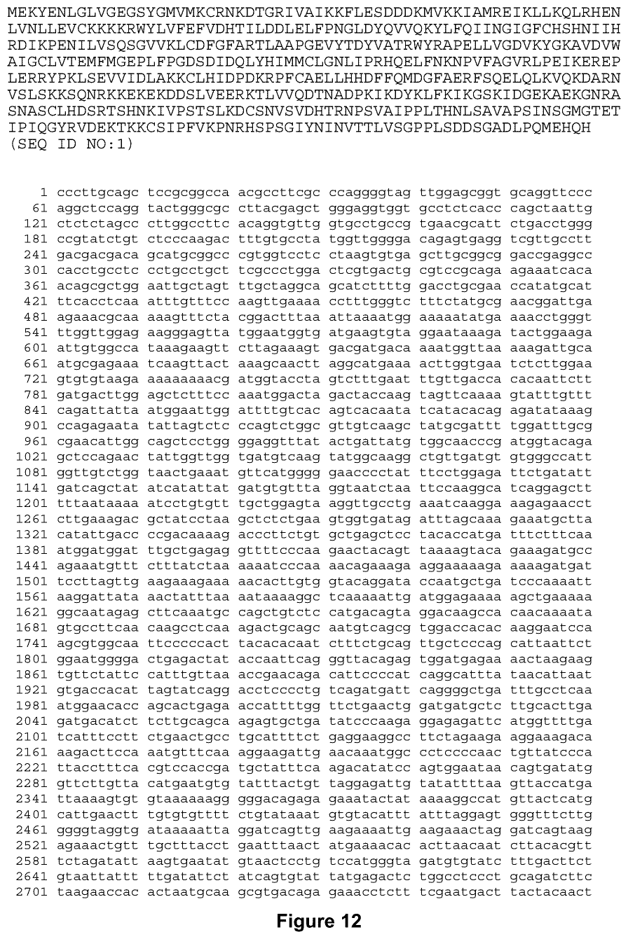

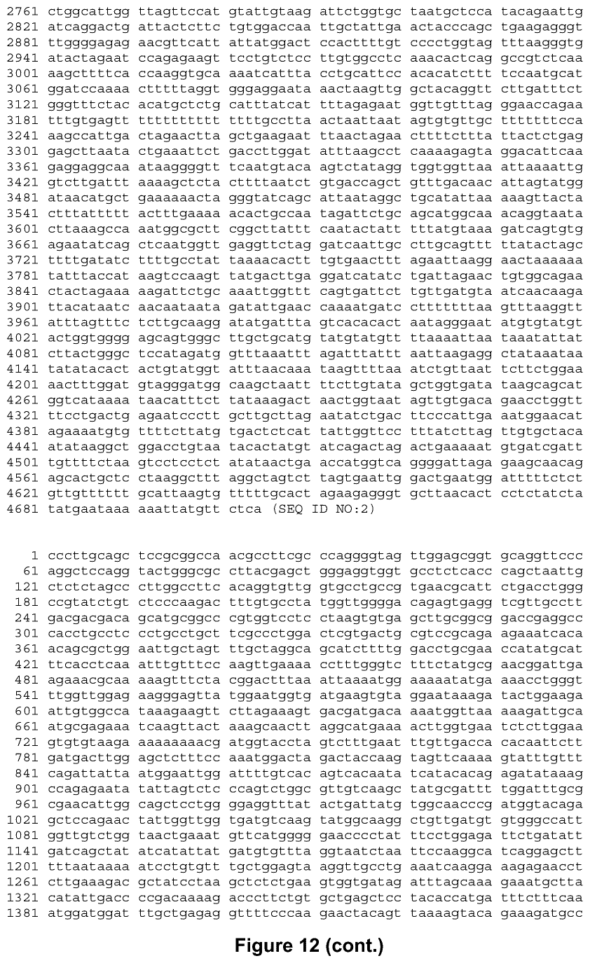

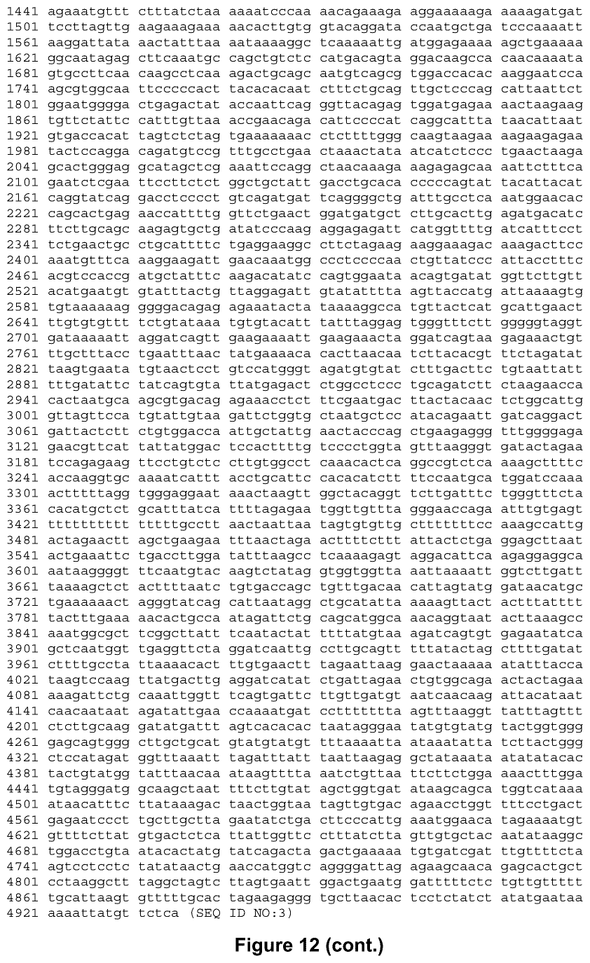

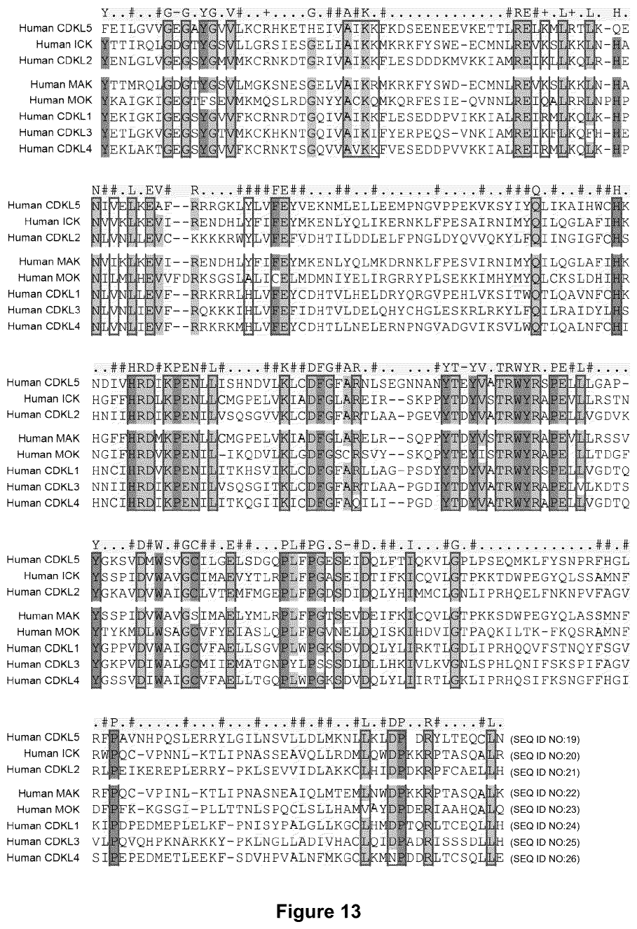

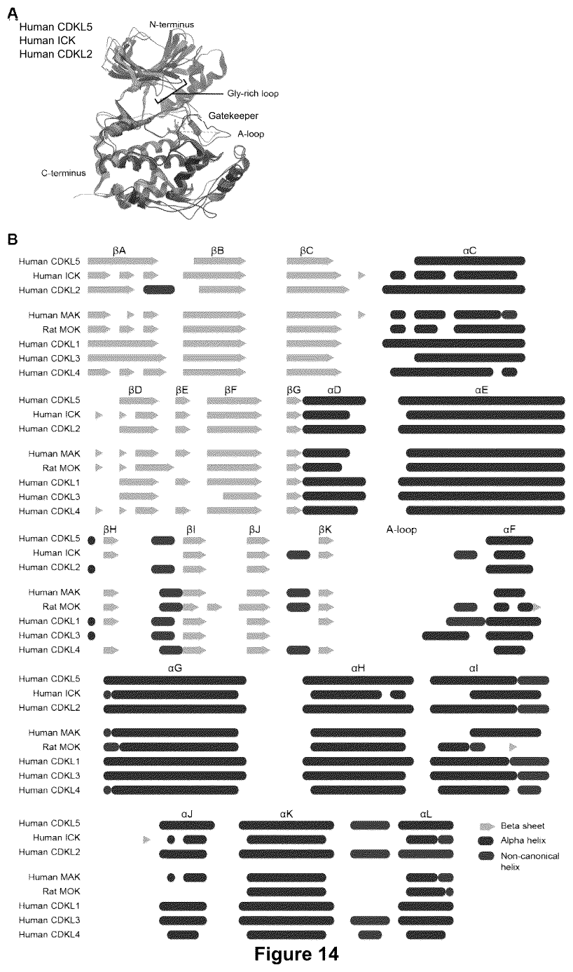

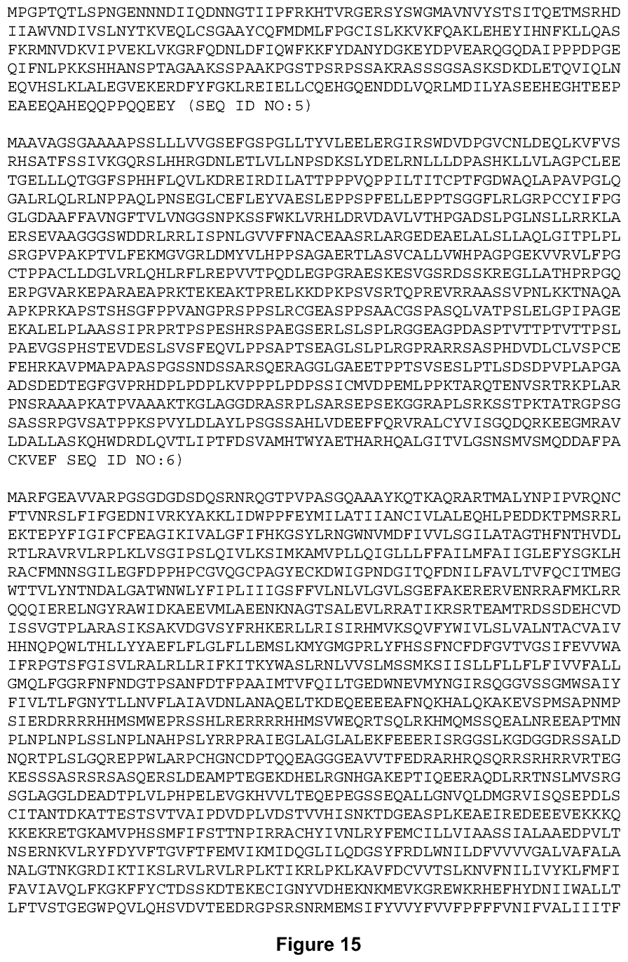

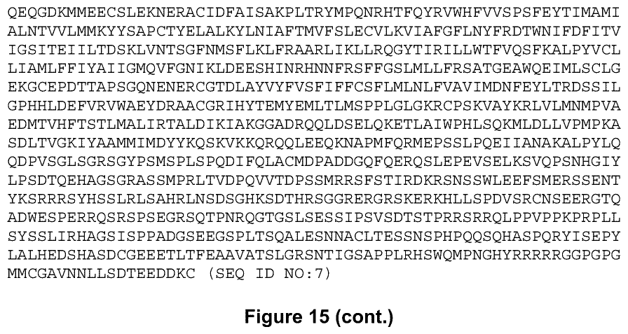

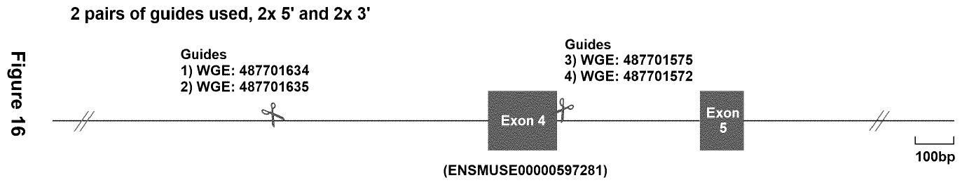

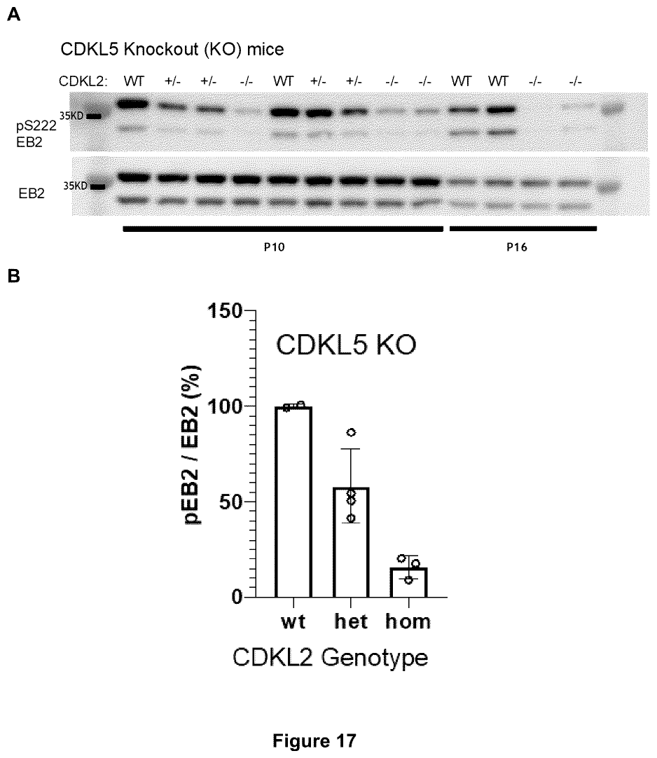

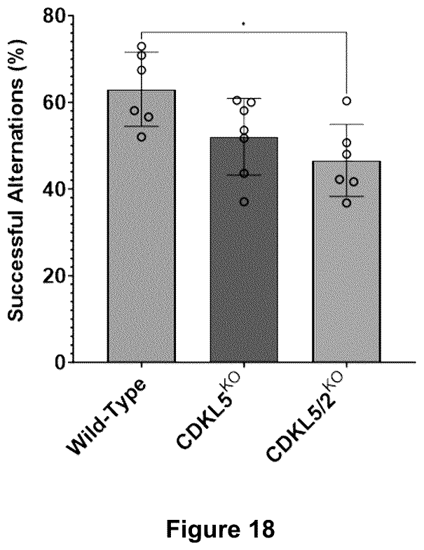

Primary neurons from CDKL5 KO mouse cortices were used to knock down CDKL2 by lentiviral expression of shRNAs targeting CDKL2. Both shRNAs caused a reduction of the OA dependent increase of pEB2/EB2. Data indicates that CDKL2 is the other kinase responsible for EB2 phosphorylation in CDKL5 KO neurons (FIG. 10).

Using the public Allen Brain Institute database, single cell mRNA expression of the kinases of interest were analysed in adult human brain cells. ICK and CDKL4 expression is very low in human neurons. Therefore, CDKL2 is the most likely candidate for phosphorylating CDKL5 substrates, such as EB2, in neurons (FIG. 11).

### Example 10—Knockout Mice

CDKL5/CDKL2 dual knockout mice have been created using Crispr-Cas9 technology. Two different CDKL2 deletions were obtained, both of which delete essential exon 4.

Two pairs of guides were used:
5′ guides: WGE ID: 487701634 Guide 1-CACATAGCTTGAGAGCGTGC AGG (SEQ ID NO: 15), and WGE ID: 487701635 Guide 2-ACATAGCTTGAGAGCGTGCA GGG (SEQ ID NO: 16) and 3′ guides: WGE ID: 487701575 Guide 3-ACGTCACCGACCAGCAGTTC TGG (SEQ ID NO: 17) and WGE ID: 487701572 Guide 4-AAGTATGGCAAGTAAGCGGC AGG (SEQ ID NO: 18). Genomic deletions were confirmed by Sanger sequencing in founder the mouse.

We have tested in vivo complementation between CDKL5 and CDKL2 in mice. We have assessed functional compensation by measuring pSer222 EB2 phosphorylation in proportion to total EB2. It was reasoned that if CDKL2 phosphorylates CDKL5 targets in vivo, then remaining EB2 phosphorylation would be reduced in CDKL5/CDKL2 double knockouts when compared to CDKL5 single knockouts.

CDKL5 activity is prominent in the whole brain, therefore measurements were done using whole brain lysates. CDKL5 homozygous knockout and CDKL2 heterozygous knockout mice were crossed to each other obtain different CDKL2 genotypes in the background of CDKL5 knockout mice. We observed that there is a CDKL2 gene dose dependent reduction in EB2 pSer222. These results indicate that in vivo in mouse brain, CDKL2 is the primary compensating kinase for CDKL5 activity, as measured by phosphorylation of EB2.

CDKL5 deficiency disorder patients are expected to have functional CDKL2 genes. It is however not sufficient to compensate for the lack of CDKL5 in humans. These data further support that increasing functional CDKL2 expression or activity in human neurons could be therapeutic for CDKL5 deficiency disorder.

### Example 11—CDKL5/CDKL2 Double Knockout Mice Show Synergistic Impairment in Short-Term Memory

Methods: Adult mice were individually placed in a Y-shaped maze in low light conditions and recorded behaving for 10 minutes. Animals were used during the dark period of their light-dark cycle. Successful alternations in the maze were automatically calculated by Ethovision software.

Results: Mice are expected to explore unvisited areas more often than recently visited areas, thus spontaneous alternation of the travelled arm in the Y-maze is used as a measure of short-term working memory. Mice with good working memory will remember previously visited arms of the maze and prefer exploring new arms; less alteration implies poorer working memory.

In this experiment we compared wild type (C57BI/6) mice with CDKL5 knockout (KO) mice and CDKL5/2 knockout mice. We expected that if CDKL2 is working in parallel with CDKL5 in brain development, loss of CDKL5 and CDKL2 together would have a larger impact in brain function than CDKL5 loss alone. We found that in Y-maze, CDKL2/5 dual knockout had less % of successful alterations when compared to wild type mice. CDKL5 KO mice and wild type mice did not differ in their successful alterations significantly. These data show that CDKL2 and CDKL5 deletion together causes diminished short-term memory and provide further evidence that CDKL2 expression can compensate for loss of CDKL5 function. These results provide yet more evidence that increasing CDKL2 may be used as a therapeutic treatment option in CDKL5-deficiency disorder.

### Example 12—Increasing CDKL2 in CDKL5-KO Mice Increases Phosphorylation of CDKL5 Substrates

Methods: CDKL5-KO mice are treated with an agent that increases CDKL2 activity in brain, or a control agent. The agent is a nucleic acid molecule, a viral vector encoding CDKL2, a small molecule, a polypeptide encoding CDKL2, or an antisense oligonucleotide. Following treatment with the agent, mouse brains are harvested and pSer222 EB2 phosphorylation levels are assessed by Western blot or another method. Phosphorylation of other CDKL5 substrates including MAP1S and CACNA1E are also assessed in the same way.

Results: CDKL5-KO mice show reduced levels of EB2 phosphorylation relative to wild type. Treating CDKL5-KO mice with the CDKL2-increasing agent is expected to increase EB2 phosphorylation levels relative to CDKL5-KO mice treated with the control agent. Phosphorylation of other CDKL5 substrates including MAP1S and CACNA1E is also expected to increase in CDKL2-treated CDKL5-KO mice relative to CDKL5-KO mice treated with control.

### Example 13—Increasing CDKL2 in CDKL5-KO Mice Rescues Behavioural Defects

Methods: CDKL5-KO mice are treated with an agent that increases CDKL2 activity in brain, or a control agent. The agent is a nucleic acid molecule, a viral vector encoding CDKL2, a small molecule, a polypeptide encoding CDKL2, or an antisense oligonucleotide. Wild type mice, CDKL5-KO control-treated mice, and CDKL5-KO CDKL2 activity increasing agent-treated mice are assessed on behavioural assays including the Y maze, open field test, T maze, fear memory, light-dark exploration, object recognition test, radial arm maze, hindlimb clasping and Barnes maze.

Results: CDKL5-KO control-treated mice are expected to perform worse on behavioural assays relative to wild type mice. CDKL5-KO CDKL2 increasing agent-treated mice are expected to perform better than CDKL5-KO control-treated mice.

Summary of results: CDKL5-deficiency disorder (CDD) is caused by mutations in CDKL5 leading to loss of the protein or a reduction of its kinase activity and reduced phosphorylation of CDKL5 substrates. CDKL5 is X-linked. Due to X-chromosome inactivation, in cells expressing the CDKL5 gene that contains the loss of function mutation(s), increasing CDKL5 gene expression (via targeting CDKL5 at DNA or mRNA level) is not a viable treatment option for CDD (since this would only increase the expression of the faulty gene). As an alternative, the inventors have identified CDKL2 as a potential therapeutic target for CDD. The results described above demonstrate that CDKL2 and CDKL5 are expressed in the same brain cell types and act on the same substrates. In particular, both CDKL5 and CDKL2 phosphorylate EB2 in neuronal cells. Given this shared activity, and the close phylogenetic relationship of CDKL2 and CDKL5, increasing CDKL2 levels in CDD presents a viable treatment option to compensate for loss of CDKL5 function, restore normal neural activity in CDD brain, and rescue behavioural defects.

It will be apparent to those skilled in the art that various modifications and variations can be made to the compositions and processes of this invention. Thus, it is intended that the present invention cover such modifications and variations, provided they come within the scope of the appended claims and their equivalents.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Further embodiments of the present invention are described below.
1. A compound or composition for increasing cyclin dependent kinase like 2 (CDKL2) expression or activity.
2. The compound or composition according to embodiment 1, wherein the compound or composition comprises a nucleic acid molecule, for example a small interfering RNA (siRNA), a guide RNA (gRNA), or a short hairpin RNA (shRNA).
3. The compound or composition according to embodiment 1 or embodiment 2, wherein the compound or composition comprises a nucleic acid molecule encoding a CDKL2 polypeptide.
4. The compound or composition according to embodiment 3, wherein the nucleic acid molecule encoding CDKL2 encodes a polypeptide having at least 80%, at least 85%, at least 90%, at least 95% or at least 99% identity to a polypeptide of SEQ ID NO:1.
5. The compound or composition according to embodiment 3 or 4, wherein the nucleic acid molecule encoding CDKL2 encodes a polypeptide comprising the sequence of SEQ ID NO:1.
6. The compound or composition according to any one of embodiments 3 to 5, wherein the nucleic acid molecule encoding CDKL2 encodes a polypeptide consisting of the sequence of SEQ ID NO: 1.
7. The compound or composition according to any one of embodiments 3 to 6, wherein the nucleic acid molecule encoding CDKL2 comprises a nucleotide sequence having at least 80%, at least 85%, at least 90%, at least 95% or at least 99% identity to a nucleotide sequence selected from the list consisting of SEQ ID NO:2 and SEQ ID NO:3.
8. The compound or composition according to any one of embodiments 3 to 7, wherein the nucleic acid molecule encoding CDKL2 comprises a nucleotide sequence selected from the list consisting of SEQ ID NO:2 and SEQ ID NO:3.
9. The compound or composition according to any one of embodiments 3 to 8, wherein the nucleic acid molecule encoding CDKL2 consists of a nucleotide sequence selected from the list consisting of SEQ ID NO:2 and SEQ ID NO:3.
10. The compound or composition according to any one of embodiments 1 to 9, wherein the compound or composition comprises a viral vector.
11. The compound or composition according to embodiment 10, wherein the viral vector comprises one or more of an adenoviral vector, an adeno-associated viral vector, a lentiviral vector or a retroviral vector.
12. The compound or composition according to any one of embodiments 2 to 11, wherein the nucleic acid molecule further comprises a promoter.
13. The compound or composition according to any one of embodiments 1 or 2, wherein the compound or composition comprises one or more of Clustered Regularly Interspaced Short Palindromic Repeat (CRISPR) associated nucleases, Transcription activator-like effector nucleases (TALEN) or Zinc finger nucleases (ZNF).
14. The compound or composition according to any one of embodiments 1, 2 or 13, wherein the compound or composition comprises a CRISPR-associated protein (Cas protein) and a guide RNA (gRNA) that hybridizes within a CDKL2 genomic sequence.
15. The compound or composition according to embodiment 14, wherein the CDKL2 genomic sequence is defined by Ensembl reference ENSG00000138769 (SEQ ID NO:8).
16. The compound or composition according to embodiment 1, wherein the compound or composition comprises an isolated CDKL2 polypeptide.
17. The compound or composition according to embodiment 16, wherein the compound or composition comprises a polypeptide having at least 80%, at least 85%, at least 90%, at least 95% or at least 99% to a polypeptide of SEQ ID NO:1.
18. The compound or composition according to embodiment 16 or embodiment 17, wherein the compound or composition comprises a polypeptide of SEQ ID NO:1.
19. The compound or composition according to any one of embodiments 16 to 18, wherein the compound or composition consists of a polypeptide of SEQ ID NO:1.
20. The compound or composition according to any preceding embodiment, wherein the compound or composition comprises or further comprises a small molecule activator of CDKL2 expression or activity.
21. The compound or composition according to any preceding embodiment, wherein the compound or composition comprises a small molecule activator that specifically binds to the protein structure defined in PDB database reference 4BBM.
22. The compound or composition according to any preceding embodiment, wherein the compound or composition comprises or further comprises a polypeptide that increases CDKL2 expression.
23. The compound or composition according to embodiment 22, wherein the compound or composition comprises a transcription factor or an enhancer that increases CDKL2 expression.
24. The compound or composition according to any preceding embodiment, wherein the compound or composition comprises or further comprises an antibody.
25. The compound or composition according to any preceding embodiment, wherein the compound or composition comprises a fusion protein comprising a CDKL2 polypeptide operatively linked to a second polypeptide sequence.
26. The compound or composition according to embodiment 25, wherein the second polypeptide sequence comprises a nuclear localization signal (NLS), a nuclear export signal (NES), a leader signal polypeptide or a cell-penetrating polypeptide.
27. The compound or composition according to any preceding embodiment wherein the compound or composition increases CDKL2 activity by at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450% or at least 500% relative to the CDKL2 activity measured in the absence of the compound or composition.
28. The compound or composition according to embodiment 27, wherein CDKL2 activity is measured by the level of phosphorylation of a CDKL2 substrate and/or the level of CDKL2 autophosphorylation.
29. The compound or composition according to embodiment 27 or embodiment 28, wherein CDKL2 activity is measured by the level of phosphorylation of microtubule-associated protein RP/EB family member 2 (MAPRE2/EB2), microtubule-associated protein 1S (MAP1S), or voltage-dependent R-type calcium channel subunit alpha-1E (CACNA1E).
30. The compound or composition according to embodiment 29, wherein the microtubule-associated protein RP/EB family member 2 (MAPRE2/EB2) comprises or consists of the amino acid sequence of SEQ ID NO:5, the microtubule-associated protein 1S (MAP1S) comprises or consists of the amino acid sequence of SEQ ID NO:6, or the voltage-dependent R-type calcium channel subunit alpha-1E (CACNA1E) comprises or consists of the amino acid sequence of SEQ ID NO:7.
31. The compound or composition according to any one of embodiments 27 to 29, wherein CDKL2 activity is measured by the level of phosphorylation of microtubule-associated protein RP/EB family member 2 (MAPRE2/EB2), optionally wherein the microtubule-associated protein RP/EB family member 2 (MAPRE2/EB2) comprises or consists of the amino acid sequence of SEQ ID NO:5.
32. The compound or composition according to any preceding embodiment wherein the compound or composition increases CDKL2 expression by at least 10%, at least 20%, at least 30%, at least 40%, at least 50%, at least 60%, at least 70%, at least 80%, at least 90% or at least 100%, at least 150%, at least 200%, at least 250%, at least 300%, at least 350%, at least 400%, at least 450% or at least 500% relative to the CDKL2 expression measured in the absence of the compound or composition.
33. The compound or composition according to embodiment 32, wherein CDKL2 expression is measured by western blot, quantitative real-time PCR (qRT-PCR), advanced RNA sequencing, microarray, mass spectrometry, ELISA or Phos-tag gel electrophoresis.
34. The compound or composition according to any preceding embodiment further comprising a pharmaceutically acceptable carrier or excipient.
35. The compound or composition according to any preceding embodiment further comprising a second therapeutic agent useful for the treatment of CDKL5 deficiency disorder.
36. The compound or composition according to embodiment 35 wherein the second therapeutic agent is selected from the list consisting of: clobazam, topiramate, prednisolone/prednisone, adrenocorticotropic hormone and ganaxolone.
37. A method of increasing cyclin dependent kinase like 2 (CDKL2) expression or activity in a subject in need thereof comprising administering a compound or composition according to any preceding embodiment to the subject.
38. A method of treating or preventing a disease or disorder in a subject, the method comprising administering a compound or composition according to any one of embodiments 1 to 36 to the subject.
39. A method of treating or preventing a neuronal disease or disorder in a subject, the method comprising administering a compound or composition according to any one of embodiments 1 to 36 to the subject.
40. A method of treating or preventing a cyclin dependent kinase like 5 (CDKL5) deficiency disorder in a subject, the method comprising administering a compound or composition according to any one of embodiments 1 to 36 to the subject.
41. The compound or composition according to any one of embodiments 1 to 36 for use in a method of increasing cyclin dependent kinase like 2 (CDKL2) expression or activity in a subject in need thereof.
42. The compound or composition according to any one of embodiments 1 to 36 for use in a method of treating or preventing a disease or disorder in a subject.
43. The compound or composition according to any one of embodiments 1 to 36 for use in a method of treating or preventing a neuronal disease or disorder in a subject.
44. The compound or composition according to any one of embodiments 1 to 36 for use in a method of treating or preventing a cyclin dependent kinase like 5 (CDKL5) deficiency disorder in a subject.
45. Use of a compound or composition according to any one of embodiments 1 to 36 for increasing cyclin dependent kinase like 2 (CDKL2) expression or activity in a subject in need thereof.
46. Use of a compound or composition according to any one of embodiments 1 to 36 for treating or preventing a disease or disorder in a subject.
47. Use of a compound or composition according to any one of embodiments 1 to 36 for treating or preventing a neuronal disease or disorder in a subject.
48. Use of a compound or composition according to any one of embodiments 1 to 36 for treating or preventing a cyclin dependent kinase like 5 (CDKL5) deficiency disorder in a subject.
49. The method, compound or composition for use or use according to any one of embodiments 37 to 48, wherein the compound or composition is administered intrathecally, intravenously, intracistnerally, intracerebroventrically or intraparenchymally.
50. The method, compound or composition for use or use according to any one of embodiments 37 to 49, wherein the subject is a mammal.
51. The method, compound or composition for use or use according to any one of embodiments 37 to 50, wherein the subject is a human.
52. The method, compound or composition for use or use according to embodiment 50 or 51, wherein the compound or composition increases cyclin dependent kinase like 2 (CDKL2) expression or activity in the subject.
53. A compound or composition according to any one of embodiments 1 to 36 for increasing cyclin dependent kinase like 2 (CDKL2) expression or activity in a subject.
54. A composition comprising a cyclin dependent kinase like 2 (CDKL2) activator.
55. The composition according to embodiment 54 further comprising a pharmaceutically acceptable carrier or excipient.
56. The composition according to embodiment 54 or 55, wherein the composition increases CDKL2 activity by at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450% or at least 500% relative to the CDKL2 activity measured in the absence of the composition.
57. The composition according to embodiment 56, wherein CDKL2 activity is measured by the level of phosphorylation of a CDKL2 substrate and/or the level of CDKL2 autophosphorylation.
58. The composition according to embodiment 56 or embodiment 57, wherein CDKL2 activity is measured by the level of phosphorylation of microtubule-associated protein RP/EB family member 2 (MAPRE2/EB2), microtubule-associated protein 1S (MAP1S), or voltage-dependent R-type calcium channel subunit alpha-1E (CACNA1E).
59. The composition according to embodiment 58, wherein the microtubule-associated protein RP/EB family member 2 (MAPRE2/EB2) comprises or consists of the amino acid sequence of SEQ ID NO: 5, the microtubule-associated protein 1S (MAP1S) comprises or consists of the amino acid sequence of SEQ ID NO:6, or the voltage-dependent R-type calcium channel subunit alpha-1E (CACNA1E) comprises or consists of the amino acid sequence of SEQ ID NO:7.
60. The composition according to any one of embodiments 57 to 59, wherein CDKL2 activity is measured by the level of phosphorylation of microtubule-associated protein RP/EB family member 2 (MAPRE2/EB2), optionally wherein the microtubule-associated protein RP/EB family member 2 (MAPRE2/EB2) comprises or consists of the amino acid sequence of SEQ ID NO:5.
61. The composition according to any one of embodiments 54 to 60, wherein the composition increases CDKL2 expression by at least 10%, at least 20%, at least 30%, at least 40%, at least 50%, at least 60%, at least 70%, at least 80%, at least 90% or at least 100%, at least 150%, at least 200%, at least 250%, at least 300%, at least 350%, at least 400%, at least 450% or at least 500% relative to the CDKL2 expression measured in the absence of the composition.
62. The composition according to embodiment 61, wherein CDKL2 expression is measured by western blot, quantitative real-time PCR (qRT-PCR), advanced RNA sequencing, microarray, mass spectrometry, ELISA or Phos-tag gel electrophoresis.

### REFERENCES

[1] Parenti, I., L. G. Rabaneda, H. Schoen and G. Novarino (2020). “Neurodevelopmental Disorders: From Genetics to Functional Pathways.” Trends Neurosci 43 (8): 608-621.
[2] Gilissen, C., J. Y. Hehir-Kwa, D. T. Thung, M. van de Vorst, B. W. van Bon, M. H. Willemsen, M. Kwint, I. M. Janssen, A. Hoischen, A. Schenck, R. Leach, R. Klein, R. Tearle, T. Bo, R. Pfundt, H. G. Yntema, B. B. de Vries, T. Kleefstra, H. G. Brunner, L. E. Vissers and J. A. Veltman (2014). “Genome sequencing identifies major causes of severe intellectual disability.” Nature 511 (7509): 344-347.
[3] Hoy, S. M. (2019). “Onasemnogene Abeparvovec: First Global Approval.” Drugs 79 (11): 1255-1262.
[4] Evans, B. (2013). “How autism became autism: The radical transformation of a central concept of child development in Britain.” Hist Human Sci 26 (3): 3-31.
[5] Kalscheuer, V. M., J. Tao, A. Donnelly, G. Hollway, E. Schwinger, S. Kübart, C. Menzel, M. Hoeltzenbein, N. Tommerup, H. Eyre, M. Harbord, E. Haan, G. R. Sutherland, H. H. Ropers and J. Gécz (2003). “Disruption of the serine/threonine kinase 9 gene causes severe X-linked infantile spasms and mental retardation.” Am J Hum Genet 72 (6): 1401-1411.
[6] Hanefeld, F. (1985). “The clinical pattern of the Rett syndrome.” Brain Dev 7 (3): 320-325.
[7] Fehr, S., M. Wilson, J. Downs, S. Williams, A. Murgia, S. Sartori, M. Vecchi, G. Ho, R. Polli, S. Psoni, X. Bao, N. de Klerk, H. Leonard and J. Christodoulou (2013). “The CDKL5 disorder is an independent clinical entity associated with early-onset encephalopathy.” Eur J Hum Genet 21 (3): 266-273.
[8] Kothur, K., K. Holman, E. Farnsworth, G. Ho, M. Lorentzos, C. Troedson, S. Gupta, R. Webster, P. G. Procopis, M. P. Menezes, J. Antony, S. Ardern-Holmes, R. C. Dale, J. Christodoulou, D. Gill and B. Bennetts (2018). “Diagnostic yield of targeted massively parallel sequencing in children with epileptic encephalopathy.” Seizure 59: 132-140.
[9] Lindy, A. S., M. B. Stosser, E. Butler, C. Downtain-Pickersgill, A. Shanmugham, K. Retterer, T. Brandt, G. Richard and D. A. Mcknight (2018). “Diagnostic outcomes for genetic testing of 70 genes in 8565 patients with epilepsy and neurodevelopmental disorders.” Epilepsia 59 (5): 1062-1071.
[10] Olson, H. E., S. T. Demarest, E. M. Pestana-Knight, L. C. Swanson, S. Iqbal, D. Lal, H. Leonard, J. H. Cross, O. Devinsky and T. A. Benke (2019). “Cyclin-Dependent Kinase-Like 5 Deficiency Disorder: Clinical Review.” Pediatr Neurol 97: 18-25.
[11] Fehr, S., H. Leonard, G. Ho, S. Williams, N. de Klerk, D. Forbes, J. Christodoulou and J. Downs (2015). “There is variability in the attainment of developmental milestones in the CDKL5 disorder.” J Neurodev Disord 7 (1): 2.
[12] Fehr, S., K. Wong, R. Chin, S. Williams, N. de Klerk, D. Forbes, R. Krishnaraj, J. Christodoulou, J. Downs and H. Leonard (2016). “Seizure variables and their relationship to genotype and functional abilities in the CDKL5 disorder.” Neurology 87 (21): 2206-2213.
[13] Hagebeuk, E. E., C. L. Marcelis, M. Alders, A. Kaspers and A. W. de Weerd (2015). “Two Siblings With a CDKL5 Mutation: Genotype and Phenotype Evaluation.” J Child Neurol 30 (11): 1515-1519.
[14] Jakimiec, M., J. Paprocka and R. Smigiel (2020). “CDKL5 Deficiency Disorder-A Complex Epileptic Encephalopathy.” Brain Sci 10 (2).
[15] Bahi-Buisson, N., A. Kaminska, N. Boddaert, M. Rio, A. Afenjar, M. Gérard, F. Giuliano, J. Motte, D. Heron, M. A. Morel, P. Plouin, C. Richelme, V. des Portes, O. Dulac, C. Philippe, C. Chiron, R. Nabbout and T. Bienvenu (2008). “The three stages of epilepsy in patients with CDKL5 mutations.” Epilepsia 49 (6): 1027-1037.
[16] Hector, R. D., V. M. Kalscheuer, F. Hennig, H. Leonard, J. Downs, A. Clarke, T. A. Benke, J. Armstrong, M. Pineda, M. E. S. Bailey and S. R. Cobb (2017). “CDKL5 variants: Improving our understanding of a rare neurologic disorder.” Neurol Genet 3 (6): e200.
[17] Bahi-Buisson, N. and T. Bienvenu (2012). “CDKL5-Related Disorders: From Clinical Description to Molecular Genetics.” Mol Syndromol 2 (3-5): 137-152.
[18] Van Esch, H., A. Jansen, M. Bauters, G. Froyen and J. P. Fryns (2007). “Encephalopathy and bilateral cataract in a boy with an interstitial deletion of Xp22 comprising the CDKL5 and NHS genes.” Am J Med Genet A 143 (4): 364-369.
[19] Bahi-Buisson, N., B. Girard, A. Gautier, J. Nectoux, Y. Fichou, Y. Saillour, K. Poirier, J. Chelly and T. Bienvenu (2010). “Epileptic encephalopathy in a girl with an interstitial deletion of Xp22 comprising promoter and exon 1 of the CDKL5 gene.” Am J Med Genet B Neuropsychiatr Genet 153b (1): 202-207.
[20] Mei, D., C. Marini, F. Novara, B. D. Bernardina, T. Granata, E. Fontana, E. Parrini, A. R. Ferrari, A. Murgia, O. Zuffardi and R. Guerrini (2010). “Xp22.3 genomic deletions involving the CDKL5 gene in girls with early onset epileptic encephalopathy.” Epilepsia 51 (4): 647-654.
[21] Bartnik, M., K. Derwińska, M. Gos, E. Obersztyn, K. E. Kołodziejska, A. Erez, A. Szpecht-Potocka, P. Fang, I. Terczyńska, H. Mierzewska, N. J. Lohr, G. A. Bellus, T. Reimschisel, E. Bocian, T. Mazurczak, S. W. Cheung and P. Stankiewicz (2011). “Early-onset seizures due to mosaic exonic deletions of CDKL5 in a male and two females.” Genet Med 13 (5): 447-452.
[22] Szafranski, P., S. Golla, W. Jin, P. Fang, P. Hixson, R. Matalon, D. Kinney, H. G. Bock, W. Craigen, J. L. Smith, W. Bi, A. Patel, S. Wai Cheung, C. A. Bacino and P. Stankiewicz (2015). “Neurodevelopmental and neurobehavioral characteristics in males and females with CDKL5 duplications.” Eur J Hum Genet 23 (7): 915-921.
[23] Weaving, L. S., J. Christodoulou, S. L. Williamson, K. L. Friend, O. L. Mckenzie, H. Archer, J. Evans, A. Clarke, G. J. Pelka, P. P. Tam, C. Watson, H. Lahooti, C. J. Ellaway, B. Bennetts, H. Leonard and J. Gecz (2004). “Mutations of CDKL5 cause a severe neurodevelopmental disorder with infantile spasms and mental retardation.” Am J Hum Genet 75 (6): 1079-1093.
[24] https://www.rosb.org/structure/5BBM
[25] https://www.omim.org/entry/300672
[26] https://www.icd10data.com/ICD10CM/Codes/G00-G99/G40-G47/G40-/G40_42
[27] Bahi-Buisson, N., N. Villeneuve, E. Caietta, A. Jacquette, H. Maurey, G. Matthijs, H. Van Esch, A. Delahaye, A. Moncla, M. Milh, F. Zufferey, B. Diebold and T. Bienvenu (2012). “Recurrent mutations in the CDKL5 gene: genotype-phenotype relationships.” Am J Med Genet A 158a (7): 1612-1619.
[28] Stosser, M. B., A. S. Lindy, E. Butler, K. Retterer, C. M. Piccirillo-Stosser, G. Richard and D. A. Mcknight (2018). “High frequency of mosaic pathogenic variants in genes causing epilepsy-related neurodevelopmental disorders.” Genet Med 20 (4): 403-410.
[29] Baltussen, L. L., Negraes, P. D., Silvestre, M., Claxton, S., Moeskops, M., Christodoulou, E., Flynn, H. R., Snijders, A. P., Muotri, A. R. and Ultanir, S. K., 2018. Chemical genetic identification of CDKL5 substrates reveals its role in neuronal microtubule dynamics. The EMBO journal, 37 (24), p.e99763.
[30] Amendola E, Zhan Y, Mattucci C, Castroflorio E, Calcagno E, Fuchs C, Lonetti G, Silingardi D, Vyssotski A L, Farley D et al (2014) Mapping pathological phenotypes in a mouse model of CDKL5 disorder. PloS one 9: e91613

1. A compound or composition for increasing cyclin dependent kinase like 2 (CDKL2) expression or activity, for use in a method of treating or preventing a cyclin dependent kinase like 5 (CDKL5) deficiency disorder in a subject.

2. The compound or composition for use according to claim 1, wherein the compound or composition comprises a nucleic acid molecule, a small interfering RNA (siRNA), a guide RNA (gRNA), or a short hairpin RNA (shRNA).

3. The compound or composition for use according to claim 1 or claim 2, wherein the compound or composition comprises a nucleic acid molecule encoding a CDKL2 polypeptide.

4. The compound or composition for use according to claim 3, wherein the nucleic acid molecule encoding CDKL2 encodes a polypeptide having at least 80%, at least 85%, at least 90%, at least 95% or at least 99% identity to a polypeptide of SEQ ID NO:1.

5. The compound or composition for use according to claim 3 or 4, wherein the nucleic acid molecule encoding CDKL2 comprises a nucleotide sequence having at least 80%, at least 85%, at least 90%, at least 95% or at least 99% identity to a nucleotide sequence selected from the list consisting of SEQ ID NO:2 and SEQ ID NO:3.

6. The compound or composition for use according to any one of claims 3 to 5, wherein the nucleic acid molecule encoding CDKL2 comprises a nucleotide sequence selected from the list consisting of SEQ ID NO:2 and SEQ ID NO:3.

7. The compound or composition for use according to any one of claims 1 to 6, wherein the compound or composition comprises a viral vector.

8. The compound or composition for use according to claim 7, wherein the viral vector comprises one or more of an adenoviral vector, an adeno-associated viral vector, a lentiviral vector or a retroviral vector.

9. The compound or composition for use according to any one of claims 2 to 8, wherein the nucleic acid molecule further comprises a promoter.

10. The compound or composition for use according to any one of claim 1 or 2, wherein the compound or composition comprises one or more of Clustered Regularly Interspaced Short Palindromic Repeat (CRISPR) associated nucleases, Transcription activator-like effector nucleases (TALEN) or Zinc finger nucleases (ZNF).

11. The compound or composition for use according to any one of claim 1, 2 or 10, wherein the compound or composition comprises a CRISPR-associated protein (Cas protein) and a guide RNA (gRNA) that hybridizes within a CDKL2 genomic sequence.

12. The compound or composition for use according to claim 11, wherein the CDKL2 genomic sequence is defined by Ensembl reference ENSG00000138769 (SEQ ID NO:8).

13. The compound or composition for use according to claim 1, wherein the compound or composition comprises an isolated CDKL2 polypeptide.

14. The compound or composition for use according to claim 13, wherein the compound or composition comprises a polypeptide having at least 80%, at least 85%, at least 90%, at least 95% or at least 99% to a polypeptide of SEQ ID NO:1.

15. The compound or composition for use according to any preceding claim, wherein the compound or composition comprises or further comprises a small molecule activator of CDKL2 expression or activity.

16. The compound or composition for use according to any preceding claim, wherein the compound or composition comprises or further comprises a polypeptide that increases CDKL2 expression.

17. The compound or composition for use according to any preceding claim wherein the compound or composition increases CDKL2 activity by at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450% or at least 500% relative to the CDKL2 activity measured in the absence of the compound or composition.

18. The compound or composition for use according to claim 17, wherein CDKL2 activity is measured by the level of phosphorylation of a CDKL2 substrate and/or the level of CDKL2 autophosphorylation.

19. The compound or composition for use according to claim 17 or claim 18, wherein CDKL2 activity is measured by the level of phosphorylation of microtubule-associated protein RP/EB family member 2 (MAPRE2/EB2), microtubule-associated protein 1S (MAP1S), or voltage-dependent R-type calcium channel subunit alpha-1E (CACNA1E).

20. The compound or composition for use according to any preceding claim wherein the compound or composition increases CDKL2 expression by at least 10%, at least 20%, at least 30%, at least 40%, at least 50%, at least 60%, at least 70%, at least 80%, at least 90% or at least 100%, at least 150%, at least 200%, at least 250%, at least 300%, at least 350%, at least 400%, at least 450% or at least 500% relative to the CDKL2 expression measured in the absence of the compound or composition.

21. The compound or composition for use according to claim 20, wherein CDKL2 expression is measured by western blot, quantitative real-time PCR (qRT-PCR), advanced RNA sequencing, microarray, mass spectrometry, ELISA or Phos-tag gel electrophoresis.

22. The compound or composition for use according to any preceding claim further comprising a pharmaceutically acceptable carrier or excipient.

23. A compound or composition as defined in any of claims 1 to 22 for use in a method of treating or preventing a neuronal disease or disorder in a subject.

24. A compound or composition for increasing cyclin dependent kinase like 2 (CDKL2) expression or activity in the brain of a subject.

25. A recombinant adeno-associated virus (AAV) particle comprising an AAV capsid protein and a nucleic acid payload, wherein the nucleic acid payload encodes a polypeptide having at least 80%, at least 85%, at least 90%, at least 95% or at least 99% identity to a polypeptide of SEQ ID NO: 1, and wherein the recombinant AAV is capable of crossing the blood-brain barrier.

26. A recombinant adeno-associated virus (AAV) particle comprising an AAV capsid protein and a nucleic acid payload, wherein the nucleic acid payload encodes a polypeptide of SEQ ID NO: 1, and wherein the recombinant AAV is capable of crossing the blood-brain barrier.

27. A recombinant adeno-associated virus (AAV) particle comprising an AAV capsid protein and a nucleic acid payload, wherein the nucleic acid payload comprises a nucleotide sequence having at least 80%, at least 85%, at least 90%, at least 95% or at least 99% identity to a nucleotide sequence selected from the list consisting of SEQ ID NO:2 and SEQ ID NO:3, and wherein the recombinant AAV is capable of crossing the blood-brain barrier.

28. A recombinant adeno-associated virus (AAV) particle comprising an AAV capsid protein and a nucleic acid payload, wherein the nucleic acid payload comprises a nucleotide sequence selected from the list consisting of SEQ ID NO:2 and SEQ ID NO:3, and wherein the recombinant AAV is capable of crossing the blood-brain barrier.

29. A compound or composition for increasing cyclin dependent kinase like 2 (CDKL2) expression or activity, wherein the compound or composition is conjugated to a targeting agent.

30. The compound or composition of claim 29, wherein the targeting agent is an agent that targets the brain.

31. The compound or composition of claim 29 or 30, wherein the targeting agent is an agent that crosses the blood-brain barrier.

32. The compound or composition of any of claims 29-31, wherein the compound or composition is a peptide, a protein, or a small molecule.


SEQUENCE LISTING (26 sequences)

SEQ ID NO: 1 - protein, 493 aa, Homo sapiens
MEKYENLGLVGEGSYGMVMKCRNKDTGRIVAIKKFLESDDDKMVKKIAMREIKLLKQLRHENLVNLLEVCKKKKRWYLVFEFVDHTILDDLELFPNGLDYQVVQKYLFQIINGIGFCHSHNIIHRDIKPENILVSQSGVVKLCDFGFARTLAAPGEVYTDYVATRWYRAPELLVGDVKYGKAVDVWAIGCLVTEMFMGEPLFPGDSDIDQLYHIMMCLGNLIPRHQELFNKNPVFAGVRLPEIKEREPLERRYPKLSEVVIDLAKKCLHIDPDKRPFCAELLHHDFFQMDGFAERFSQELQLKVQKDARNVSLSKKSQNRKKEKEKDDSLVEERKTLVVQDTNADPKIKDYKLFKIKGSKIDGEKAEKGNRASNASCLHDSRTSHNKIVPSTSLKDCSNVSVDHTRNPSVAIPPLTHNLSAVAPSINSGMGTETIPIQGYRVDEKTKKCSIPFVKPNRHSPSGIYNINVTTLVSGPPLSDDSGADLPQMEHQH

SEQ ID NO: 2 - RNA, 4704 nt, Homo sapiens
  source 1..4704: mol_type = mRNA
cccttgcagctccgcggccaacgccttcgcccaggggtagttggagcggtgcaggttcccaggctccaggtactgggcgccttacgagctgggaggtggtgcctctcacccagctaattgctctctagcccttggccttcacaggtgttggtgcctgccgtgaacgcattctgacctgggccgtatctgtctcccaagactttgtgcctatggttggggacagagtgaggtcgttgccttgacgacgacagcatgcggcccgtggtcctcctaagtgtgagcttgcggcggaccgaggcccacctgcctccctgcctgcttcgccctggactcgtgactgcgtccgcagaagaaatcacaacagcgctggaattgctagtttgctaggcagcatcttttggacctgcgaaccatatgcatttcacctcaaatttgtttccaagttgaaaacctttgggtctttctatgcgaacggattgaagaaacgcaaaaagtttctacggactttaaattaaaatggaaaaatatgaaaacctgggtttggttggagaagggagttatggaatggtgatgaagtgtaggaataaagatactggaagaattgtggccataaagaagttcttagaaagtgacgatgacaaaatggttaaaaagattgcaatgcgagaaatcaagttactaaagcaacttaggcatgaaaacttggtgaatctcttggaagtgtgtaagaaaaaaaaacgatggtacctagtctttgaatttgttgaccacacaattcttgatgacttggagctctttccaaatggactagactaccaagtagttcaaaagtatttgtttcagattattaatggaattggattttgtcacagtcacaatatcatacacagagatataaagccagagaatatattagtctcccagtctggcgttgtcaagctatgcgattttggatttgcgcgaacattggcagctcctggggaggtttatactgattatgtggcaacccgatggtacagagctccagaactattggttggtgatgtcaagtatggcaaggctgttgatgtgtgggccattggttgtctggtaactgaaatgttcatgggggaacccctatttcctggagattctgatattgatcagctatatcatattatgatgtgtttaggtaatctaattccaaggcatcaggagctttttaataaaaatcctgtgtttgctggagtaaggttgcctgaaatcaaggaaagagaacctcttgaaagacgctatcctaagctctctgaagtggtgatagatttagcaaagaaatgcttacatattgaccccgacaaaagacccttctgtgctgagctcctacaccatgatttctttcaaatggatggatttgctgagaggttttcccaagaactacagttaaaagtacagaaagatgccagaaatgtttctttatctaaaaaatcccaaaacagaaagaaggaaaaagaaaaagatgattccttagttgaagaaagaaaaacacttgtggtacaggataccaatgctgatcccaaaattaaggattataaactatttaaaataaaaggctcaaaaattgatggagaaaaagctgaaaaaggcaatagagcttcaaatgccagctgtctccatgacagtaggacaagccacaacaaaatagtgccttcaacaagcctcaaagactgcagcaatgtcagcgtggaccacacaaggaatccaagcgtggcaattcccccacttacacacaatctttctgcagttgctcccagcattaattctggaatggggactgagactataccaattcagggttacagagtggatgagaaaactaagaagtgttctattccatttgttaaaccgaacagacattccccatcaggcatttataacattaatgtgaccacattagtatcaggacctcccctgtcagatgattcaggggctgatttgcctcaaatggaacaccagcactgagaaccattttggttctgaactggatgatgctcttgcacttgagatgacatcttcttgcagcaagagtgctgatatcccaagaggagagattcatggttttgatcatttccttctgaactgcctgcattttctgaggaaggccttctagaagaaggaaagacaaagacttccaaatgtttcaaaggaagattgaacaaatggccctccccaactgttatcccattacctttcacgtccaccgatgctatttcaagacatatccagtggaataacagtgatatggttcttgttacatgaatgtgtatttactgttaggagattgtatattttaagttaccatgattaaaagtgtgtaaaaaagggggacagagagaaatactataaaaggccatgttactcatgcattgaactttgtgtgttttctgtataaatgtgtacatttatttaggagtgggtttcttgggggtaggtgataaaaattaggatcagttgaagaaaattgaagaaactaggatcagtaagagaaactgtttgctttacctgaatttaactatgaaaacacacttaacaatcttacacgtttctagatattaagtgaatatgtaactcctgtccatgggtagatgtgtatctttgacttctgtaattatttttgatattctatcagtgtattatgagactctggcctccctgcagatcttctaagaaccacactaatgcaagcgtgacagagaaacctctttcgaatgacttactacaactctggcattggttagttccatgtattgtaagattctggtgctaatgctccatacagaattgatcaggactgattactcttctgtggaccaattgctattgaactacccagctgaagagggtttggggagagaacgttcattattatggactccacttttgtcccctggtagtttaagggtgatactagaatccagagaagttcctgtctccttgtggcctcaaacactcaggccgtctcaaaagcttttcaccaaggtgcaaaatcatttacctgcattccacacatcttttccaatgcatggatccaaaactttttaggtgggaggaataaactaagttggctacaggttcttgatttctgggtttctacacatgctctgcatttatcattttagagaatggttgtttagggaaccagaatttgtgagttttttttttttttttgccttaactaattaatagtgtgttgctttttttccaaagccattgactagaacttagctgaagaatttaactagaacttttctttattactctgaggagcttaatactgaaattctgaccttggatatttaagcctcaaaagagtaggacattcaagaggaggcaaataaggggtttcaatgtacaagtctataggtggtggttaaattaaaattggtcttgatttaaaagctctacttttaatctgtgaccagctgtttgacaacattagtatggataacatgctgaaaaaactagggtatcagcattaataggctgcatattaaaaagttactactttatttttactttgaaaaacactgccaatagattctgcagcatggcaaacaggtaatacttaaagccaaatggcgcttcggcttatttcaatactatttttatgtaaagatcagtgtgagaatatcagctcaatggttgaggttctaggatcaattgccttgcagtttttatactagcttttgatatcttttgcctattaaaacactttgtgaactttagaattaaggaactaaaaaatatttaccataagtccaagttatgacttgaggatcatatctgattagaactgtggcagaactactagaaaaagattctgcaaattggtttcagtgattcttgttgatgtaatcaacaagattacataatcaacaataatagatattgaaccaaaatgatcctttttttaagtttaaggttatttagtttctcttgcaaggatatgatttagtcacacactaatagggaatatgtgtatgtactggtggggagcagtgggcttgctgcatgtatgtatgttttaaaattaataaatattatcttactgggctccatagatggtttaaatttagatttatttaattaagaggctataaataatatatacactactgtatggtatttaacaaataagttttaaatctgttaattcttctggaaaactttggatgtagggatggcaagctaattttcttgtatagctggtgatataagcagcatggtcataaaataacatttcttataaagactaactggtaatagttgtgacagaacctggttttcctgactgagaatcccttgcttgcttagaatatctgacttcccattgaaatggaacatagaaaatgtgttttcttatgtgactctcattattggttcctttatcttagttgtgctacaatataaggctggacctgtaatacactatgtatcagactagactgaaaaatgtgatcgatttgttttctaagtcctcctctatataactgaaccatggtcaggggattagagaagcaacagagcactgctcctaaggctttaggctagtcttagtgaattggactgaatggatttttctctgttgttttttgcattaagtgtttttgcactagaagagggtgcttaacactcctctatctatatgaataaaaaattatgttctca

SEQ ID NO: 3 - RNA, 4935 nt, Homo sapiens
  source 1..4935: mol_type = mRNA
cccttgcagctccgcggccaacgccttcgcccaggggtagttggagcggtgcaggttcccaggctccaggtactgggcgccttacgagctgggaggtggtgcctctcacccagctaattgctctctagcccttggccttcacaggtgttggtgcctgccgtgaacgcattctgacctgggccgtatctgtctcccaagactttgtgcctatggttggggacagagtgaggtcgttgccttgacgacgacagcatgcggcccgtggtcctcctaagtgtgagcttgcggcggaccgaggcccacctgcctccctgcctgcttcgccctggactcgtgactgcgtccgcagaagaaatcacaacagcgctggaattgctagtttgctaggcagcatcttttggacctgcgaaccatatgcatttcacctcaaatttgtttccaagttgaaaacctttgggtctttctatgcgaacggattgaagaaacgcaaaaagtttctacggactttaaattaaaatggaaaaatatgaaaacctgggtttggttggagaagggagttatggaatggtgatgaagtgtaggaataaagatactggaagaattgtggccataaagaagttcttagaaagtgacgatgacaaaatggttaaaaagattgcaatgcgagaaatcaagttactaaagcaacttaggcatgaaaacttggtgaatctcttggaagtgtgtaagaaaaaaaaacgatggtacctagtctttgaatttgttgaccacacaattcttgatgacttggagctctttccaaatggactagactaccaagtagttcaaaagtatttgtttcagattattaatggaattggattttgtcacagtcacaatatcatacacagagatataaagccagagaatatattagtctcccagtctggcgttgtcaagctatgcgattttggatttgcgcgaacattggcagctcctggggaggtttatactgattatgtggcaacccgatggtacagagctccagaactattggttggtgatgtcaagtatggcaaggctgttgatgtgtgggccattggttgtctggtaactgaaatgttcatgggggaacccctatttcctggagattctgatattgatcagctatatcatattatgatgtgtttaggtaatctaattccaaggcatcaggagctttttaataaaaatcctgtgtttgctggagtaaggttgcctgaaatcaaggaaagagaacctcttgaaagacgctatcctaagctctctgaagtggtgatagatttagcaaagaaatgcttacatattgaccccgacaaaagacccttctgtgctgagctcctacaccatgatttctttcaaatggatggatttgctgagaggttttcccaagaactacagttaaaagtacagaaagatgccagaaatgtttctttatctaaaaaatcccaaaacagaaagaaggaaaaagaaaaagatgattccttagttgaagaaagaaaaacacttgtggtacaggataccaatgctgatcccaaaattaaggattataaactatttaaaataaaaggctcaaaaattgatggagaaaaagctgaaaaaggcaatagagcttcaaatgccagctgtctccatgacagtaggacaagccacaacaaaatagtgccttcaacaagcctcaaagactgcagcaatgtcagcgtggaccacacaaggaatccaagcgtggcaattcccccacttacacacaatctttctgcagttgctcccagcattaattctggaatggggactgagactataccaattcagggttacagagtggatgagaaaactaagaagtgttctattccatttgttaaaccgaacagacattccccatcaggcatttataacattaatgtgaccacattagtctctagtgaaaaaaacctcttttgggcaagtaagaaaagaagagaatactccaggacagatgtccgtttgcctgaactaaactataatcatctccctgaactaagagcactgggaggcatagctcgaaattccaggctaacaaagaaagagagcaaaattctttcagaatctcgaattccttctctggctgctattgacctgcacacccccagtattacattacatcaggtatcaggacctcccctgtcagatgattcaggggctgatttgcctcaaatggaacaccagcactgagaaccattttggttctgaactggatgatgctcttgcacttgagatgacatcttcttgcagcaagagtgctgatatcccaagaggagagattcatggttttgatcatttccttctgaactgcctgcattttctgaggaaggccttctagaagaaggaaagacaaagacttccaaatgtttcaaaggaagattgaacaaatggccctccccaactgttatcccattacctttcacgtccaccgatgctatttcaagacatatccagtggaataacagtgatatggttcttgttacatgaatgtgtatttactgttaggagattgtatattttaagttaccatgattaaaagtgtgtaaaaaagggggacagagagaaatactataaaaggccatgttactcatgcattgaactttgtgtgttttctgtataaatgtgtacatttatttaggagtgggtttcttgggggtaggtgataaaaattaggatcagttgaagaaaattgaagaaactaggatcagtaagagaaactgtttgctttacctgaatttaactatgaaaacacacttaacaatcttacacgtttctagatattaagtgaatatgtaactcctgtccatgggtagatgtgtatctttgacttctgtaattatttttgatattctatcagtgtattatgagactctggcctccctgcagatcttctaagaaccacactaatgcaagcgtgacagagaaacctctttcgaatgacttactacaactctggcattggttagttccatgtattgtaagattctggtgctaatgctccatacagaattgatcaggactgattactcttctgtggaccaattgctattgaactacccagctgaagagggtttggggagagaacgttcattattatggactccacttttgtcccctggtagtttaagggtgatactagaatccagagaagttcctgtctccttgtggcctcaaacactcaggccgtctcaaaagcttttcaccaaggtgcaaaatcatttacctgcattccacacatcttttccaatgcatggatccaaaactttttaggtgggaggaataaactaagttggctacaggttcttgatttctgggtttctacacatgctctgcatttatcattttagagaatggttgtttagggaaccagaatttgtgagttttttttttttttttgccttaactaattaatagtgtgttgctttttttccaaagccattgactagaacttagctgaagaatttaactagaacttttctttattactctgaggagcttaatactgaaattctgaccttggatatttaagcctcaaaagagtaggacattcaagaggaggcaaataaggggtttcaatgtacaagtctataggtggtggttaaattaaaattggtcttgatttaaaagctctacttttaatctgtgaccagctgtttgacaacattagtatggataacatgctgaaaaaactagggtatcagcattaataggctgcatattaaaaagttactactttatttttactttgaaaaacactgccaatagattctgcagcatggcaaacaggtaatacttaaagccaaatggcgcttcggcttatttcaatactatttttatgtaaagatcagtgtgagaatatcagctcaatggttgaggttctaggatcaattgccttgcagtttttatactagcttttgatatcttttgcctattaaaacactttgtgaactttagaattaaggaactaaaaaatatttaccataagtccaagttatgacttgaggatcatatctgattagaactgtggcagaactactagaaaaagattctgcaaattggtttcagtgattcttgttgatgtaatcaacaagattacataatcaacaataatagatattgaaccaaaatgatcctttttttaagtttaaggttatttagtttctcttgcaaggatatgatttagtcacacactaatagggaatatgtgtatgtactggtggggagcagtgggcttgctgcatgtatgtatgttttaaaattaataaatattatcttactgggctccatagatggtttaaatttagatttatttaattaagaggctataaataatatatacactactgtatggtatttaacaaataagttttaaatctgttaattcttctggaaaactttggatgtagggatggcaagctaattttcttgtatagctggtgatataagcagcatggtcataaaataacatttcttataaagactaactggtaatagttgtgacagaacctggttttcctgactgagaatcccttgcttgcttagaatatctgacttcccattgaaatggaacatagaaaatgtgttttcttatgtgactctcattattggttcctttatcttagttgtgctacaatataaggctggacctgtaatacactatgtatcagactagactgaaaaatgtgatcgatttgttttctaagtcctcctctatataactgaaccatggtcaggggattagagaagcaacagagcactgctcctaaggctttaggctagtcttagtgaattggactgaatggatttttctctgttgttttttgcattaagtgtttttgcactagaagagggtgcttaacactcctctatctatatgaataaaaaattatgttctca

SEQ ID NO: 4 - DNA, 180 bp, synthetic construct
  source 1..180: mol_type = other DNA
ttctgcttgcttctaggcgcggctaagtcaagtcctgcatctaagccaggatccacacctgctcgccccgcggctgccaaaagggcttcatccagtggctctgcatccagatctgacaaagatttagaaacgcaggtcatacagctcaacgagcaggtaattcatcgcctccacccttgt

SEQ ID NO: 5 - protein, 327 aa, Homo sapiens
MPGPTQTLSPNGENNNDIIQDNNGTIIPFRKHTVRGERSYSWGMAVNVYSTSITQETMSRHDIIAWVNDIVSLNYTKVEQLCSGAAYCQFMDMLFPGCISLKKVKFQAKLEHEYIHNFKLLQASFKRMNVDKVIPVEKLVKGRFQDNLDFIQWFKKFYDANYDGKEYDPVEARQGQDAIPPPDPGEQIFNLPKKSHHANSPTAGAAKSSPAAKPGSTPSRPSSAKRASSSGSASKSDKDLETQVIQLNEQVHSLKLALEGVEKERDFYFGKLREIELLCQEHGQENDDLVQRLMDILYASEEHEGHTEEPEAEEQAHEQQPPQQEEY

SEQ ID NO: 6 - protein, 1059 aa, Homo sapiens
MAAVAGSGAAAAPSSLLLVVGSEFGSPGLLTYVLEELERGIRSWDVDPGVCNLDEQLKVFVSRHSATFSSIVKGQRSLHHRGDNLETLVLLNPSDKSLYDELRNLLLDPASHKLLVLAGPCLEETGELLLQTGGFSPHHFLQVLKDREIRDILATTPPPVQPPILTITCPTFGDWAQLAPAVPGLQGALRLQLRLNPPAQLPNSEGLCEFLEYVAESLEPPSPFELLEPPTSGGFLRLGRPCCYIFPGGLGDAAFFAVNGFTVLVNGGSNPKSSFWKLVRHLDRVDAVLVTHPGADSLPGLNSLLRRKLAERSEVAAGGGSWDDRLRRLISPNLGVVFFNACEAASRLARGEDEAELALSLLAQLGITPLPLSRGPVPAKPTVLFEKMGVGRLDMYVLHPPSAGAERTLASVCALLVWHPAGPGEKVVRVLFPGCTPPACLLDGLVRLQHLRFLREPVVTPQDLEGPGRAESKESVGSRDSSKREGLLATHPRPGQERPGVARKEPARAEAPRKTEKEAKTPRELKKDPKPSVSRTQPREVRRAASSVPNLKKTNAQAAPKPRKAPSTSHSGFPPVANGPRSPPSLRCGEASPPSAACGSPASQLVATPSLELGPIPAGEEKALELPLAASSIPRPRTPSPESHRSPAEGSERLSLSPLRGGEAGPDASPTVTTPTVTTPSLPAEVGSPHSTEVDESLSVSFEQVLPPSAPTSEAGLSLPLRGPRARRSASPHDVDLCLVSPCEFEHRKAVPMAPAPASPGSSNDSSARSQERAGGLGAEETPPTSVSESLPTLSDSDPVPLAPGAADSDEDTEGFGVPRHDPLPDPLKVPPPLPDPSSICMVDPEMLPPKTARQTENVSRTRKPLARPNSRAAAPKATPVAAAKTKGLAGGDRASRPLSARSEPSEKGGRAPLSRKSSTPKTATRGPSGSASSRPGVSATPPKSPVYLDLAYLPSGSSAHLVDEEFFQRVRALCYVISGQDQRKEEGMRAVLDALLASKQHWDRDLQVTLIPTFDSVAMHTWYAETHARHQALGITVLGSNSMVSMQDDAFPACKVEF

SEQ ID NO: 7 - protein, 2313 aa, Homo sapiens
MARFGEAVVARPGSGDGDSDQSRNRQGTPVPASGQAAAYKQTKAQRARTMALYNPIPVRQNCFTVNRSLFIFGEDNIVRKYAKKLIDWPPFEYMILATIIANCIVLALEQHLPEDDKTPMSRRLEKTEPYFIGIFCFEAGIKIVALGFIFHKGSYLRNGWNVMDFIVVLSGILATAGTHFNTHVDLRTLRAVRVLRPLKLVSGIPSLQIVLKSIMKAMVPLLQIGLLLFFAILMFAIIGLEFYSGKLHRACFMNNSGILEGFDPPHPCGVQGCPAGYECKDWIGPNDGITQFDNILFAVLTVFQCITMEGWTTVLYNTNDALGATWNWLYFIPLIIIGSFFVLNLVLGVLSGEFAKERERVENRRAFMKLRRQQQIERELNGYRAWIDKAEEVMLAEENKNAGTSALEVLRRATIKRSRTEAMTRDSSDEHCVDISSVGTPLARASIKSAKVDGVSYFRHKERLLRISIRHMVKSQVFYWIVLSLVALNTACVAIVHHNQPQWLTHLLYYAEFLFLGLFLLEMSLKMYGMGPRLYFHSSFNCFDFGVTVGSIFEVVWAIFRPGTSFGISVLRALRLLRIFKITKYWASLRNLVVSLMSSMKSIISLLFLLFLFIVVFALLGMQLFGGRFNFNDGTPSANFDTFPAAIMTVFQILTGEDWNEVMYNGIRSQGGVSSGMWSAIYFIVLTLFGNYTLLNVFLAIAVDNLANAQELTKDEQEEEEAFNQKHALQKAKEVSPMSAPNMPSIERDRRRRHHMSMWEPRSSHLRERRRRHHMSVWEQRTSQLRKHMQMSSQEALNREEAPTMNPLNPLNPLSSLNPLNAHPSLYRRPRAIEGLALGLALEKFEEERISRGGSLKGDGGDRSSALDNQRTPLSLGQREPPWLARPCHGNCDPTQQEAGGGEAVVTFEDRARHRQSQRRSRHRRVRTEGKESSSASRSRSASQERSLDEAMPTEGEKDHELRGNHGAKEPTIQEERAQDLRRTNSLMVSRGSGLAGGLDEADTPLVLPHPELEVGKHVVLTEQEPEGSSEQALLGNVQLDMGRVISQSEPDLSCITANTDKATTESTSVTVAIPDVDPLVDSTVVHISNKTDGEASPLKEAEIREDEEEVEKKKQKKEKRETGKAMVPHSSMFIFSTTNPIRRACHYIVNLRYFEMCILLVIAASSIALAAEDPVLTNSERNKVLRYFDYVFTGVFTFEMVIKMIDQGLILQDGSYFRDLWNILDFVVVVGALVAFALANALGTNKGRDIKTIKSLRVLRVLRPLKTIKRLPKLKAVFDCVVTSLKNVFNILIVYKLFMFIFAVIAVQLFKGKFFYCTDSSKDTEKECIGNYVDHEKNKMEVKGREWKRHEFHYDNIIWALLTLFTVSTGEGWPQVLQHSVDVTEEDRGPSRSNRMEMSIFYVVYFVVFPFFFVNIFVALIIITFQEQGDKMMEECSLEKNERACIDFAISAKPLTRYMPQNRHTFQYRVWHFVVSPSFEYTIMAMIALNTVVLMMKYYSAPCTYELALKYLNIAFTMVFSLECVLKVIAFGFLNYFRDTWNIFDFITVIGSITEIILTDSKLVNTSGFNMSFLKLFRAARLIKLLRQGYTIRILLWTFVQSFKALPYVCLLIAMLFFIYAIIGMQVFGNIKLDEESHINRHNNFRSFFGSLMLLFRSATGEAWQEIMLSCLGEKGCEPDTTAPSGQNENERCGTDLAYVYFVSFIFFCSFLMLNLFVAVIMDNFEYLTRDSSILGPHHLDEFVRVWAEYDRAACGRIHYTEMYEMLTLMSPPLGLGKRCPSKVAYKRLVLMNMPVAEDMTVHFTSTLMALIRTALDIKIAKGGADRQQLDSELQKETLAIWPHLSQKMLDLLVPMPKASDLTVGKIYAAMMIMDYYKQSKVKKQRQQLEEQKNAPMFQRMEPSSLPQEIIANAKALPYLQQDPVSGLSGRSGYPSMSPLSPQDIFQLACMDPADDGQFQERQSLEPEVSELKSVQPSNHGIYLPSDTQEHAGSGRASSMPRLTVDPQVVTDPSSMRRSFSTIRDKRSNSSWLEEFSMERSSENTYKSRRRSYHSSLRLSAHRLNSDSGHKSDTHRSGGRERGRSKERKHLLSPDVSRCNSEERGTQADWESPERRQSRSPSEGRSQTPNRQGTGSLSESSIPSVSDTSTPRRSRRQLPPVPPKPRPLLSYSSLIRHAGSISPPADGSEEGSPLTSQALESNNACLTESSNSPHPQQSQHASPQRYISEPYLALHEDSHASDCGEEETLTFEAAVATSLGRSNTIGSAPPLRHSWQMPNGHYRRRRRGGPGPGMMCGAVNNLLSDTEEDDKC

SEQ ID NO: 8 - DNA, 55421 bp, Homo sapiens
  source 1..55421: mol_type = genomic DNA
aatggggtaagctctgttcctgtgtgatttaagcctgtgcatgattctgacctaccagtcttgctcagtcgtggattgagctgcatttttgcatttaaggcaagttttatcgcaagccctaactccaccccatttggctgccatacgcgggtgttgtgcgtgtcctccagatccccagccatcccagcgctttcccattccactcatgccctctcttccccttcctccatctcagtgttttccctcactttcaatggccagactggccttcccaaggtccctcctaacgtcaggtctgtcatcacctaccttcttccaccttgcctcgcctactaatcatcacctcctaaactgtcaccgttatctcctacctggctcacactggccccaccgcccagaacagaaaggaagcatggaatcccggtcaggccgcgcctacgcgactcagggcgcccggcgcccgcccctgccccagcggcgatgctatgagggaaaccgtatcgcattttgcatagtcttcgcagtcctacataaccgccacctttacccttcgcgtgggcatcacaatcgcctcctcccgctggggaaggcagaaaggcgcctcctgacgagaaccaaggcgtgtggggacgcagggcctctgcgtgtcagggagcagaacctgggccgagccctaggtgaaggggcggggtggttggccctgagccaatcatggctcgtgacgactcggctcggccaatcagaagaagggaggcctggcgctctcggggcgggtgagaaaccgcccccccttgcagctccgcggccaacgccttcgcccaggggtagttggagcggtgcaggttcccaggctccaggtactgggcgccttacgagctgggaggtggtgcctctcacccagctaattgctctctagcccttggccttcacaggtgttggtgcctgccgtgaacgcattctgacctgggccgtatctgtctcccaagactttgtgcctatggttggggacagagtgaggtcgttgccttgacgacgacagcatgcggcccgtggtcctcctaagtgtgagcttgcggcggaccgaggcccacctgcctccctgcctgcttcgccctggactcgtgactgcgtccgcagaagaaatcacaacagcgctggaattgctagtttgctaggcagcatcttttggacctgcgaaccatatgcatttcacctcaaatttgtttccaagttgaaaacctttgggtctttctatgcgaacggattgaagaaacggtaagtatagtacgttttaaagcttctattatctcgttctttgatctataaaagatttcaatcttttttttttttttttttttttttttttttttttttttttgagacggagtttcactcttgttgcccaggctggagtgcaatggcatggtctcggctcaccgcaacctctgcctcctgggttcaagcgattatcctgcctcagcctcccaagtagctgggattacaggcatcaccaccacgcccggctaagtttgtatttttagtagagacagggtttctccatgttggttaggctggtctcgaactcgcaacctcaggtgatccgcccgcctcggcctcccaaagtgttgggattacaggcgtgagccaccgctcccggccctttaatctgtcttaaacaaaataaaaaattagcattctccaaatatttttatcaaaattgttgcttttagaaccatccagaaataacaccttgcccattggccaggtttctttttcgtttgtattacctttccgttcaagagtagaaggcatgagaagttatagtttatgagtaactgtttcaggaaacaggaggctttgtaggctgtgatacttttaaagtatttgtggacagtgtcacttgaaaaagcaggtgtgttcaatgtggtgtgtccagatatgcaattggttgggagaataaacttgaagcagtcaacttgcattaatgttagcttgggatatgcttcagaacacctcagacatgcttaaagtcattttttttggttcactttgtgctttcaaatattgaggacatcaattacaaaatgttttaaaataatccaggttatagattcaagctatttttgtcattggtatgataaacctaaagcctagtgcttaataaaacatactctcttaatggaagagaaaaaaattaagaagggatagaattatatggagattttttttttcaaatacaaacagtttattaaaaaatagttaatggaagtggaatagaaggtaaaatgtcaaatgcatggaaagttagtgtctgggacactgctctgtaaaaagtttgtagtagaatctactatgaaaactggtagtaaaataataatttaagtcgatgttcattggtattgttttcatttcccacccctcaagatcaatcttctggttctccacatatagctacattaaaaatatatatattataaaatctcaaactattatatgtcaagtgtggaaaacgtattgtatacattggattagcatagtttgctagaacaaagagaatatttgttttgtctttgcatcactgttccaatagagaaataggaaaaacagttcacgatctgagtaaattttagattgccgtaagtatacaattaatacaacaggcaagaattatttcttgttaaaatctatctgtatttggactgaaataattttttatctattattactgaaataatagatagttaaaatctatctgtatttggactgaaataatttttttctgtacgaaagcattttgtccaatgattgctggaccttcgtcaatcattggtaaaacaagaaagattctcttaggattgcctttattcagaaaatgtcatataatatgagtaaaaaatttcaaggccgggcgcggtggctcacgcctgtaatcccagaactttcggaggccgaggcgggcagatcacgaggtcaggagatcgatacaatcctggctaacacggtgaaaccccgtctctactaaaaatacaaaaagttagctgggcgtggtggcaggcgcctgtagtcccagctactcgagaggttgaggcaggagaatggcgtggacccgggaggcggagtttgcagtgagccaagatcgcgccactgcactccagcctgggcgacagagcaagactccgtctcaaaaaaaaaaaaaaaatttcaaacacaaattcaaattatacttatagtgagtaatcattaacagggtttttgaaatacttttactgtacttggtactactaatatttaatttaatttaataattacattaaatttaataaaatttaatttaattttaaaaatctgcttatataccgatgtcattactcttattttctaagttcaatatagtaacagtaacatggcgaaaccccgtctctactaaaagtacaaaaattagctgggcgtggtagcggtgcctgtaaccctagctactggggaggcagaaacagggaatcgcttgaacctgggaggcagaggttgcagtgagccgagatcgcgccagtgcacgagactccgtttcaaaaaaaaaaaaaaaaaaaagaaaaaaaaagtaaagaaaaatattttatgaaagatgctctgtgttaatttgttatgtgatcaaatattttttccaattaagtggaaggtgcataagtatttccaacattgatctaaatccagtgtaacaaaaaagttgatctttattaaaactaaattcaggccgggcgctctggctcacgcctgcaatcccagcactttgggaggccaaggtgggtggatcacttgaggtcaggagtttgagatcagcctgggcaacatggcaaaaccccatctctatgaaaatacaaaacttacccgggtgtggtggctcacgcctataatcccagctactcagaggcggaggcaggagaattgcttgaacccaggaggtggaggttgccgtgagcctaaatctcaccactgcactccagcctgggcaacagagcaagactccgtcccaaacaaacaaacaaacaaacaaacaaaatcccccaccaaattcagttatacatctttgagtgaaaaggaattgtgcttttttttttttgagacagagtctcactctgtcacccaggctggagtgcaatagtgctatcttggctcactgcaacctccgcctcctgggttcaagggattctcctgccttagcctcccgagtagctgggactataggggtgcaccaccatgcctggctaatttttgtattttagtagagacagggtttcaccatgttggccaggctggtctcaaactcctgacctcaggtgatctgcccgcctccgccttctaaagtgctgggattacaggtgtgagccactgcacctggcctgtgcattttgtttatttgttttgttttgtttttgagacagattcttgctccgttgctcaggctggtgtgcagtgccatcacagctcactgcagcctcaacctcctgggctcaagcaattctcctgcctcagcctcccaagtagctgggaccacaggcatacaccaccacacccagctaatttaaaaatgtcttttgtagagacagagtctcagtatgttgcccaggctggaattgtgcttttttttttttttttgagatggagtctcactctgttgcccaggctggagtgcagtggtgtgatcttggctcactgcaagctccgcctcccgggttcacgccattctcctgcctcagcctcccgagtagctgggactacaggcgcctgccaccaagtccggctaatttttctgtatttttagtagagacggggtttcaccgtgttagccaggatggtctcaatctcccgaccttgtgatccgcccaccttggcctcccaaagtgttgggattacaggcgtgagccactgcgcccggtctggaattgtgctttttaaagtgagttgtaattgttatgtgtgatagctcaagtattgctttattggaattaaaaatatatggaatttcattaacatgacatttagtaatttaaagttaatggcttcaattccatttgcgttgtctattatgaaatgtgtgtgaccattttgaaaacatagcttaatgtgtgtgaccattttgaaacatagcttacatgtagaaacactaggaaattgtgaattaatacaggacagccatgaacacagtgactaatatggaaaacaagcagggaagaattaatttgctgggaaggcggaggaaattaacgtactcaactttgttaaatctatgttttggttttcacagcaaaaagtttctacggactttaaattaaaatggaaaaatatgaaaacctgggtttggttggagaagggagttatggaatggtgatgaagtgtaggaataaagatactggaagaattgtggccataaagaagttcttagaaagtgacgatgacaaaatggttaaaaagattgcaatgcgagaaatcaagttactaaaggtgagtaaatgcatatattgaatcaaaaaatatgagtataagtttcttttttggcaataagaagaaatacaatgattagatatgcaaattcatttatctggtgaatgtaaacctgattttaaaagtgtttgttccttctctctatatctttctgtgtatttatcaatcatctatctatagtctgtgttaggagtcccaaagaccaccccagatttgatgacttgttaggaggaatagcattaatttttgttctttattatttcattttttctacttatttgtatgggtttattttgcagcccttttccctaatttgttgagatagatgcttctctaatttccagacttttttcccctcaaatatgcatttaaggatatacattttcctgtaggaacagcttggtatgtatcctggaaattttgattgttaacaatattttctcatttctcttatatttttcttaatccctgagttatttaaaagtatatttctgtatttccaatcatggacattttctaggttattctttcctggttatttatttgtgagacaactgccctatgttctgagtatacaccatataagaatttatttctttgaaatttcttgatatttgctttttgttcagaatatagtcaactaaaaaaaaccctcttgttcttgaaacgtatttcatatatgttcatgttaattgtgttgttcaaatcttccaaattcttatttattttccttacatgcttgttttatccaacactgagaagaaatatgttaaagtcttcctttgtgattattgatttttctacttttcattgtagttcagttaatgtttatttttataaacagaaacagtattattagtgtgttatatttagaattattatatcttgctgttgagttaaatcttttgttattatgcaataaccctttttattatagtaatattttgtttcgttggttttttttgagacggagtctcactctgttaccaggctggagtgcaatggtgctatctcagttcactacaacctcctcctcccgggttcaagtgatgcccctgcctcagcctcccaagcagctgggactacaggtgtgcatcaccatgcctggataatttttttttttttttgtattttagtagagacggagttgcaccatgttgattgggatggtctcgatctcctgacctcatgatccgcccacctcagcctcccaaagtgctgggattacaggtgtgagccaccacacccggtcgtttcattttgtttttttgagacagggtcttgctctgtcctccaggctggagtgcagtggcatgatcatggctcactgcagccttgaactcctgggctcaagcaatcctacacctcagtctcccaagtagctgggattgcaggtgtgtgccgctatgcctggctaatttttttattttcagtagagataaggtctcactgtgttgctcaggctggtctctaactcctgagctaaagtaatcctcctacctcggcctttcaaagtgttcagattataggtgtgagccaccacacctggccagtattttgttttaagatctaatttatttgaaattaataagagtacatgcattagcttaattttggttagcgtttccatagtaaatcttttccatatttttacttctaatttttctgaattcttttttttttttttttgagatggagtttcacacttgtcatctaggctggagtgcagtggcgtaatcttggctcactgcaacctctgcctcctgggttcaagcaattctgcctcagcctcctgagtagctggtactataggcgcccgccaccacgcctggctaatttttgtatttttagtagggatggggtttcaccatgttggccaggctggtctcaaactcctgacctcaggttatccgcctgcctcagccttccaaagtgctgagattacaggtgtaagccaccgcgcctggccccctaatttttctgaattcttgtttagatgtatctcttgtgaaaaccgtatagttatattttgcttttttcctgtttgataatatttcccttttaattggagcatttattccatttacttgtaatataatatctgatgtattttgaatatatatatctaccaccttaatatgtgctatttgtattaccagttctctatttctttgtcttttgttacttggattgattacatttatcattcctttttttctaataacttggaaattttacattgtgtttctgtcttttagtggttacactagggatttgttaaagtctaattatctaatacttgttcttgatcttaataatggtgtttgacacagtcaaatcttatgacttttcccttatggctatccagttgtcccagaaacatttattgaaaataccatctttttccccactgttctgaatattccttttgccacacgttctcctatataggtaagattttattctgtcccattggtctatttgtttaagcttgcgccagtagcccacaatcatgtttattctagttagtaggtttttttttgagacagggttttgctgtatcacccgggctgcagtgcagtggcatgatcttgactcattgcaatctctgcctcccaggctcaagtgattctcccacttcagccttctgagtagctgggcctacaggcgtgtgccaccatgcttagctaatatttgtatttttttgggtaaagatagggtttttccatgttgcccaggctagtcttgagctgcttggctcaagcaatccgccctcctcagccttccaaagtgctgggattacaggtgtgagccactgcagctggcctagtttgtaagtttttatatctggtagaataaatccttctgttttgtccttcttctataagaccatcttccctatttttgtctgtttgcatttccatataaattttagaatcaatttaccaatgttcataaacactctaggaatttattgaatttatacaatcaatttgtggagaattgatgtgtttacaatattgcatccagttcatgaacattgtataatccttttgtttcttggtcttcttgtctgtttttttttttttttttttttttttttttgatggagtcttgctctgtcacccaggctggagtacagtggtgcaatctcgtctcgctgcaacttctacccgctgggttcaagcaattctcctgcctcagcctcccaagtagctgggattacaggtgcccgccaccacacctggctaatttttgtattttagtagagacggggtttcaccatgtcggccaggctggtctcaaactcctgacctcgtgatccagccactttggcttcccaaagtgttgggattacaggcatgagccaccaggcctggccagtcttcttgtcttaacatacacttcgtcattagtatgtttgcacttttgatcattatttatatgatatatactttttaatcctttctcctattaaccttctgtgtcgttagatgtaaagtatatctcttataagcagatacagttatttcttttgtttattcagtctgacaatctttgtattttacttggagtgtttggtctatttatatttaatataaaacttacatatttggatttatacttctcttcctagttgccttccatatatctaggttttttctttttaaaaattattttgttaaacattcttttgctggtgttttagagattgttctagatattttaatatacaaacttgacttattgatactcaaacaggtacttttacatgcaaaaatcttagaaattttaacttcagttaccccctcctttctcccctccacccatcagatatatgtgttattgctgctatgtaacattgttaaattctgtgtatagttaaatcccttttaaaattgttcacagtcagcagttattaggatatacctaatattaccctttctattactctttaattttccctacatttctgagtttccatctggggtcaatttccttatgcttaaagcacttcctttattttccttaaagcaaatctgatgataagaaattctattcatttttgttagtgtaaaaaaaagtatgtattctactttgtgtttaaaatactatttcaagctcggtgcgatagcgtgggcctatagtctttgctattcaggaggctgaggtgggaggatcacttgagcccaggagtttgagactgtggtgtgctatgatcatgcctgtgaatagccactgcactccagcctgggcactgtagtgagatctcatcgcttcaaaaaaaaagaaaaaaaggccgttttctcaggatatagcattcagggttgtcagttaattacattcagtatgttcaagatagaatcctactgttctgggctccgttgcttgtattgaaaaaatcagacatcagtcttactgtgttccttaataaccattttcttctagatacttttaaggttttctctttggttttcaggagtttacaataatatgcctaggtgtgggtttttttttttttttgtctgttaattttatggctcttgcttctatcaaataatgtcatttgtcattttaaaaaaatgctgggcatggtggctcatgtctgttaatctcagcactttgggaggccaaggtgggcggatcacttgaggtcaggggttcgagaccagcctggccaacgtggtgaaaccctgtccttactaaaaatacaaaaattagctgggcatgttggtgcgcgcctatagtcccagctactcaggaggctgaggcaggagaatctcttgaacctcgcaggcagaggttgaagtaaaccaagatcatgccactgcactccagccttggcaacagagtaagactccatctcaaaaaaaaaaaaaaatgctcagctataattgttgcttcacatactgctactgtcccatttaagaattcagcttcatatactaagattcatatcagctactactgatagcttgaggacttttagggctaacccaaatgaaaggatttcaccagcactgtactccttcttttcaccttcacggcccctgaacgtcaatcactgctaccttaggcctgcaaaatggtgaaaagtgctactgatcctcttagtcttccctctaaagtggcatatgtttcttgggttaaaaaaaatttggccctaattgcctggctcacttttctgggtttctgtcttcctagcccaggtattttttactatattatctttttcgttcctccaagtagttatgttatatattttgtccagcttttctacttgtattcagttggagaattgttacaaattaccaagtctactgttactgtgaacaaaagtaattatacttttctatcctagaatttttatttgggtccacatctgttatgttacttttcatggttttaagttccctccattttttgaggttggctttcttttttaaagagtagtagtccatatctgataattacaatatctgaagtcattctgtatctgtttcaatcgccgatatgttctggtatttcttacttatggtgtcttatttcattgtgtgcctgcttatcttcaattttgtgctgaaccttatctttgaaatactattattatcattattattattttttagacagggtcttgctctgtcacccaggctggagtgcagtggtgcaattatggctcacttcagcctcaatcttccagattcaagcagtcctcctgcctcagcttcctaagtagctttgaagcttggactacaggcatgtgccaccatgcccagctaatttttttaattttttgtggagacagggtctcagaatgttgcccagactagtctcaaactactgagctcaagcaatttttctgcctcaacctacccaaatgctagaatcataggcgggagccaccacgcctgtcctgaaagattactttttaaaaataatttgaggctaaggtaaaaatatttttctccacagacaatttttatttcattcttccatgttcatgggcactgtgccagtaatcaatttattgtctctcaactccaaatcactccttttttttacctactttgtgatattagaactggactctgtaaacatttcttttttattgcccgctgatttgattttaggttttgtcaacagatagagggcatggaaagacattgtcggtccttagcaagaggaagagacttttcttccagcttctggtgtgctttttttttttttttttttttttttatacagctgtgagcccatcaccatgtgggaggtctgttagctcacttcagcatctttcaagtaccagttctggcctacctgcacattctggtgagtttctctaccaactgctggccactctacaaaccatctctggcctgtgtgctctggtgaattttttcactattggcaatctttagatttttgtggctgctgggccatttccaatgaggactatgtgtctggtggttattgggggctcttctacatttttagttccttcttgtccctatgtattttccttcagccctaaaagtggggcttcctgtatttgctagtttaaacatatcccttacagtcttcttttaccccctttagtaattaactacccttcataatgaaaaatatttacattaaaattttcccagttctggtttctgtctgatacaagcacactaccagctcaggacctccttagtaagttcaaagttcttgcttatagtctctttttatcttgtgtttctcattatcctttagaattgaatgttgtttttgaaattttatttgtagaaatattttgaggctaaggtagggttttctttgtccagaggtgatattcatttgcttctgacaggtacatggagatgctcccagtccagtatgtgaatccatgttcaaggcttttagttccttgaacgtgatgttaaattaagtctagtgatgcctcgtttatttctgttcattttcactctggaggatgtagttttttgggtcctagctttaattggaggtggtttctcagatatcccacctttagaaggcttttggactttggcttttcccttttagccctggggagccacaaaaatcaaagctcatttccacacttgttacttgatgtcaaaattagctttgagtcccgaggttaactctctggattctcatgttcttgattttagcccagtaatcctcactgtcgttttagccttttatatgtttaagaagattttttgaaaattaatccaacttttaaagaattttcaatggctgggcgcggcaactcacgcttgtaatcccagcactttgggaggccaaggcgggcggatcacctaaggttgggagttccagaccagcctgaccaacacggagaaaccccgtctctactaaaaatacaaaattagctgggcatggtggcgcatgtctgtaatcccagctattcgggaggctgaggcaggagaattgcttgaacctggcaggcggaggaggttgcgatgagccgagatcgcgccattgcactccagcctgggcaacaagagcgaaactccgtctcaaaaaaaaaaaaaaaaaaaaaaaaaaaaagaattttcaattggaggcctggtctgagttactcagtctgccattaccaaagaaggtaaccatctgccattaaaaatataagcacaaagacagagaacctgtggagctgatagtctgtgctttttttttttttttttttgagacagagtctcgctctgttgcccaggttggagtgcagtggcacgatctcggctcactgcaacctccgcctcctgggttcaagcgattctcatgcctcagcctcctgagtagctgggactacaggcacctgccaccacgcctggctaatttttgtattttcagtagagacagggattcaccattttggccaggctgatcttgaactcctgacctcgtgatccgtccgccttggcatcccaaagtgctgggattacaggcatgagccaccatgcccggccgatagtctgttttaattatgattgcaggtttggagctactgtggaaatctttgcaagaaggacttgacatagagtagagaaagataaattatctggtgctatagtgtgaatttttaaagagtaaaaagttttagtgtaatgttttcttctattagaaatgtgccaaaacagcccaatggaacaagtgacattctagtattatttagatttcttaatttgataaaatagaacttgtcagaatttatagttccattaggatatgaaatttgtgggttttggggcatttttgttggtttgtttagtttttttggtttttttgagaaggtgttttaatcatgcctctaccagttattgctgtgtgaccttggacaagttccttgacctctgtgtgagttagttgcttcagctataaagcaggataataattgtacctatttcataaggcttttgtgcttagaatagtacctggaacatagtaagcattgtgtaagtattagttattattatgtataatatcatactcttttataacttttatttttttcttggcaacgcttcttttacattgtaaaatgtctgttttaagggaaggcagggtagaagtttgtgtttttttttaacttttaagttcaggggtacatgtgcaggatgtacaggtttgttacataggtaaacatgtgtcatggggtttgatgacttggagctctttccaaatggactagactaccaactagttcaaaagtacttgtttcagattattaataatggaattggattttgtcgcagtcacgatgtaagtcttgggtttaaacaattatttgtcagtcatttttcctggtcctctcccttctcccaccctttgccctccggtaggccccagtgtgtgttgttcccctctatgtgcactcatcatttagctcccacttataagtgacaacatgtggtatttggttttctgttcctattagtttgctgaggataatggcttctagctccatccatgtccctgcaaaggacatgatctcattcttttttatggctgcataatattctatgatgtataactctttaaaaaacatttttctatatgatatttgaattcctttttttttttttttttggagatggagtcttgctctgtcacccaggctggagtgcagtggtgcaatcttagctcactgcaacttccgcctcctgggttcaagcgattctcctgcctcagcctccttagaaactgggattacttcattgatattacttcattacttcatggtggtggccaggcatgccaccacgcctgggtaatttttttgtatttttggtagagatggggttttgccatgttggccaggttggtcttgaactcctgacctcaggtgatccacctgcctcagcctcccaaagtgctgggattacaggtgtgagccactgtgcccggcccctgaattccttatattatttttatatttatattggatatgtttccttggtaatcttttttcattttttaattttttaagagagtctcactctgccccacccaggctggaatgcagtggcatgatcatagcttattgcagcctacaattcctggcctcaagctatccctccacttcagcctcccaaagcgctgggattacaggcataagctaccatgcctagccatattgcttaccttgataactatagtgttaaataacacagttacttctttgtaaattaattattctatttctatttcattttctctcccactcacctaccatctgattttagaaaatatcttatttactttagtgttttgttttttcattttgtttcaagataaggtcttgctctgttgctcgagctgaagtgcagtggcatgatcatggctcaccacactcattgcagcctccaactcgtgggcttaagtgattctcccacattaacctcccaagtagctgggactagaggtgtgcccaaccacaccaggctaatttttaaaattttttgtagagatggggtctcaccatgcccaggctggtctcaaattcctggcctcaagtgattctcttgctttgtcctcccaagtcactgggattacaggtgtgagccactgcacctggtgtatatatacacttttatcttgtttacatcttctatatcatttgtgcattgttgcatttaaaatgttcactttctgtttcataaccatgatgcccacatttgtataagatttagtcctgccttaaatggattcaatgctcatgttgtttttacttgaatgctcattcatgcttttacttgaatgacagtttggttgggtataaagttcctggatcatacttggattttcttgaggattttgagagctgtattctgcattccattggtttctttccctttaaaggtggcaacatttttttctggtgagtgttaatctaccatttatgtttctttctcctttcttccccatttcttacatctttgaatagatccttgattggttcctttttgattgtttcttataataaagtctgagtctttggaggactatttgtaggaggttctggggagaggccatgactttgttccaggctagcaggaatttaccataattcacaatggtgttccttatcacatatgcctcagtgtgggatttaattctattaggtttaatttctctaaaacttgcagttgcaggcttgttcataatgaattgttttgccttctacactgcctcactgagagattgtttccagcaaatgtgtttctgtgtgattccttatccagcccttccccaatgctgtttcttggtgccattcagttctagggatgttttcactgctgtctatgagccttatttcatatatatatagtgtatatatatatatgaatatacatatatatatatactatatatatgtatacatagttttccattatattaaaggaggacttttttgtcttttcagggtatgtcactggcttcagagaactcttacgcttccagctttgtagccacagatgatcttttggtgccttcctatactcctgtttgaccgtgattagatttagtagtcccatctcatttgttgtgagtagggggtcatagatccctcttagttttagcaaagatggagttacacttatttttcttattagctttattttgttctctgagaggagaaaggagcaatatagtctctcaattgtagtttaaaaaccagaatatgcttttacaatttacactatgtatgcattttatttatatatttttcttacgctgagatctattttaaataattaattgctaataatttgatcttagtttatataaactattttgcatttttaaataacagctattttgcattttttaataacagcaacttaggcatgaaaacttggtgaatctcttggaagtgtgtaagaaaaaaaaacgatggtacctagtctttgaatttgttgaccacacaattcttgatgacttggagctctttccaaatggactagactaccaagtagttcaaaagtatttgtttcagattattaatggaattggattttgtcacagtcacaatgtaagtattgggtttaaataatttgctttatcacatatttattcattaatcctttaatctgtcttattgatttatgcatttgaaaataagttgcagacatcagtatactttaacactaaatacttcatctgtgcatctattaattggagttaaatatttgttttctgagttttttgtttgtttgttttgtttttttccgagacagagtctcgctctttcgcccaggctggagtgcagtggcaagatctcagctcactgcaacctctgccccccaggttcaagtgattctcttgcctcagcctcctgagtagctgggattacaggcatgcaccactacgcctggctaatttttgtattcttagtagagatggggtttcaccatgttggccaggctggtctcaaaactcctgacctcaggtgatctgcctgccttggcctcccaaagtgctgggattacagccatgagccaccgtgcctggctctattttctgttcttttttaaggtaatatttactaagcgaaatgcacaaattttaagtgatgaattttgagaaatgtatagacccaagggtctgtgtatttgttagtttgtgttctcttgcctaattacaaagtgtgaatgtttgaaaatttctacccttaccctgtagtctatcacaataattttatatactttgagtatcatataaaagcacttacaagaagaatgatacctataaagtataaatgaatagctcaacacatagattttgccagttttacaaaatattttaaatgacattctttgtgtgattataagagtaataaatattaatggtagaaaaatttaaaactaaagataattataaaagaaagaaaaaatcattttacatacctgccacctaaattgatatatttcttttcggttattattattattcactgtctctaattttttaactgtcttttattgtgtagttttgttatgttactgagttatagtgtgaagtttacatacataaaaaaattgctcagtagaggcagatacctagtttccactgggaaaaacactgcccttttttaggaggccttccatagtggcctcctggcaactttttaatgatccaggctcagaaaatctatttttttttgaaacggagtctccctctatcgcccaggctggagtgcagtggcgcgatctcggctcacggcaagctccgcctcccaggctcacgccattctccggcctcagcctccggagtagctgggactacaggcgcccgccaccacgcccggagaatttttttatatttttagtagagacggggtttcaccttgttagccaggatgatctcgatcttctgacctcgtgatccgcctgccttggcctcccaaagtactgggattacaggcgtgagccaccgctcccggcccagaaaatctattttgagaaacctaattttgagactatctgtctcttagggcagactcatattgaccaaactcaggagactgaatgccagtttagtaaattgcaaagagagataattgttacggggtgaaatatataaagtagaataacaataatgttggatttgcaggaaatcagatcagcatgaggtgttagtgaatcaaatacttaatgtatcagtttaaaagatactgattcatccttcttcataaaaatgatgatatcttcccaatgggcctgtaatatttggagttaggtggctgaatgccctacattcatctctgtactaaggataggcttgccagtggaaccagtaagcaaaaagaaccattgtctctctcacattagaagcgcatttgaatgtgtcctgcctacattgcttctgtgtccctttatttttatactttcatgatgaatcaacccatgtttaatataacacattctacctttagatgtacagatacatccaggcagtttagagtcaagtactgctggcatgagtgccattctgtgtttaaggaaagagattttaggcctggtgtggtggctcatgcatgtaatcctagcactttgtggggccgacgtggtctgatcacttgaggtcaggagttcgaagccagcctggccaacgtggtgaaaccccatctctactaaaaatataaaaaaattagccgggcatggtggcaggcacctgtaatcccagctgcttgggaggctgaggcaagagaattgcttggacccaggaggcggaggttgcagtgagctgagatcgtgccactgccctccagcctgggcaacatggcgagactctgtctcaaaaacaaaaaaaaagattttaaaatctagtgtttctccaggcacagtggctcacgcctgtaattccagcaatttgggaggccgaggcgggtggatcacctgaggtcgggagtttgagaccagcctggccaacatggtgaaaccccgtctctactaaaaatacaaaattagctgggcgtggtggcacatgcctgtaatcccagctactcggaaggctgaggcaggagaattgcttgaacctgggaggcagaggttgcggtaagctgagattgtgccattgcactccagcctgggcaacaagaacgaaactccatctcaaaaaaaaaaaaaaaaaaggtggttccctgctaagcttcagaacatttacatatctctttaaactatagaaaatgtgaatttttatgggtggagaactgatctcactctaacttatgaacacttgtctcaagggagcattagtgttattagtgttgcctggcatactactttttttccagtccatttactcttttttttttttttttttttttgagacagaatctcactctgtcacccatgctagagtgcagtggcacgatcttggctcactgcaacctctgcctcctgggttcaagcggttctcctgcctcagcctctcaagtagctgggattaccggtgtgcaccaccatgcctggctaatttttgtatttttagtagagatggggtttcgccatgttggccaggctggtcttgaacttttggcctcaagtgatcggcccacctcggcctcccaaagtgctgggattacaggcgtgtgccactgtgcccagcctccatttactcttaaactcttctagattgtgatttcttaccctctttcttctcaaaccctagtaccttttctccaatccttactcccagcccctagttaaatggagataataatagcacctatccttagggttattatgagaataaaataagttaaaacatgaaaaacatttagaatagtgcctgacacataagtattcaagtgtcagctattattaccattattattaaaggtatattttaaggttttgttttctgggcagttaacatttctctttttaatgtagacagataataacggtagtggctggaataaaacacacagatatatacatacatacatacatatatataaacacatatatgtacacataaggatgagatgcttgactttaagaaaatgagaacatttattctcaaatatattattcatttttaaatgtatctatatatgtgagcacttgaattttttaaaatataaatatgattttaatcctttatgagccagactgaataacatatttaaaaacaaatttaagattgctgcccattgaagtttgacccaaatagtacagaacattatgaggtttgtagaaccctaaacgactttttttactgaaattactaggtatcataatttatttaggttatatctagtattaaagacccatgtcagagcctacccttttcatagtactttctctggccaccccagtctgcaatgatggattttaggaaaaattcaactcatatcatcttggttatacttagaatctattgtgttacttaagttataaggtattagagttgggaagaaacatggtgatcatgcactttagccctctcatttgtgggccgagaggatagacttagaaagattgagttgtctatggctgtactgaaaaccagggactagacccaggtctcccgactcccagtttagctctttctttttttctttttctttttttttttgagatggaggctcgctctgtcgcccaggatggagtgcagtggctcgatttcggctcactgcaagctccgcctcccaggttcacgccattctccctgcctcagcctcccgagtagctgggactacaggcgcctgccaccacgcctggctaattttttgtatttttggtagagacggggtttcactatattagccaggatgtctcgatctcctgacctcgtgatccacccgcctcggtctcccaaagtgctgggattacaggtgtgagccaccgcacccggccccagtttagctctttctactacagcacaggtggtctgagactggacctgtgtcctcatcactgatatctagggtaataaagttggtagcctatacaacgtgctcagcaatggcagattgccgtttaggatgctggtcttccgaaagagttaaaacaaatctatcttttttagttttcaaaatacaatttgcttatattagagaattaggcctaataatgtcttttgcctagctgtagatttttaaatatatatatattatatatataatatatcatatatgatatatatctatataatattttttatatatatattttttgagatggagtctctgtcgcccaggctggagtgcaatggcgcaatctcggctcactgcaaccttccccctcctaggttcaagagattctcctgcctgagcctcccaagtagctgggattacaggtgcctgccatagtgcctggctaatttttgtattttgactagggacggggtttcaccatgttggccaggctggtctcaaactcctgacctcatgatccacccacctcaacctcccaaagtgctgggattacaggcgtgagccaccgcacctggccagatttttaaatattatctgaatatcagcatcctgttttattcttacaaagtctctcctttagaaattttaatcactaatgtttttgtttactctttacatttctaattgcctgatgaatgtctatacttgggtgctcagcattccacccaaaagtccaatgattaaaactaaactaattattccattaaaactaactttctctaggcttttctaattttgaaaatgaaaccaccattcttttaaattctcaggttcaaacaaaatatctacaaaatatctacgactcttctaaattctatttctttcttttctttttttttttttttcttgagatggagtctcgctctgttgcccaggctggaatacagtggtgcgatcttggctcactgcaacctccacctcctgggtacaagcaattctcctgcctcaaccactcaagtagctgggattacaagcgcgtgccagcacgcccagctacttttttgtattttagtaaagacaaggtttcaccaagttggccaggctggtcttgaactcctgacctcaagtgatctgcctgcctcggcctcccaaagtgctgggattacaggcttgagccactgcgtgcggcctattgatttttgatttattgtatttctaaactgcatttcctccttcccattcctctgagcaccaacctaggttaggcctttatcacctcatactggtcccactgctcttcttcctgcctctcgtctttctctctgatttagctcatttatggccaacatcaattttaaacaccacattgtctatcacttccatgggttcccagggtcttgccacacttcctagtatgtcagctgagatattccgcctccacttcacacaacctatactctagaaaaaacagttttctggccaggcgtggtggctcacgcctgtaatcccagcactttgggaggccgaggcaggtggatcacctgaggtcaggagtttgagaccatcctggccaacatggtgaaaccccgtctctactaaaagtacaaaaattagttgggcatggtggcaggcgcctgtaatctcagctactcgggaggctgagacagtagaatcgcttgaacccaggaggcggaggttgaagtgagccgaaatcgcgccattgcactccagcctgggcgacaagagcaagacgatgcctaaaaaaaaaaaaaaaaaaaaggtttttggcccgggcgcggtggctcacgcctgtaatctcagcacttcgggaggccgaggcgggcagatcacgaggaggtcaggagatcgagaccatcctggctaacacggtgaaacgccgtctgtactaaaaatacaaaaaattagccgggcgtggtggcaggcgccagtagtcccacctactcgggaggctgaggcaggagaatggcgtgaacccgggaggtggagcttgcagtgagccgagatcgcgccactgcactccatcctgggtgacagagcgagactccgtctcaaaaaaaaatttttttttccccacaatctcttatctatccccatttccattcttgctctccttgttacattcacttgggctgcccttctttctctttatttatctaggtcatacttagtattaaagacctatgttagagcccacccctttcataaaaatttcccctgactgcctcagcttcaatgatagctttactctcttagtcttgagagcagtacattaactgaacagttgatttagtaatttgaaaaaatatattttatccaatgtttttgattgtttaaataaaatttcaagtatagatgttctttcttgcccaactaggtcctaggttgttgagggctaggagcaactttctatgcttttgtttgtattctttatagggacaacatacaatgccctgcacatagcaggtgccccaaaataattaattttatttaacatccgtcacactctgcttcttgtctagatcatacacagagatataaagccagagaatatattagtctcccagtctggcgttgtcaagctatgcgattttggatttgcgcgaacattggcagctcctggggaggtttatactgattatgtggcaacccgatggtacagagctccagaactattggttggtgatgtcaagtatggcaagtaagacatcagtaatggggaggagtagatttttactcttgcttcttgctatagtggaatatgtttatgatatagtaaatgatccctctaatactgaaaggaaggcttgagtttcagggactatataaagtaaattgtaatcatttttggtgcttcccctttaaacattcctgtgaaacaagtatacttattttagaggtggggaaattaagtggtctattttatacagtgggttggaaatagatatatttatcacttgtaaagagaggtgaacattgattggttaaatgtgacattgttggaatggggaagatggagtaagcaagggtggaggcagaaatataattaagtaaaatgtttttaaaacataaattttcttcacaagggaattcttcaaagaagaaagggttgtaacttttactgcttttgattgaaatctttctaaaatattttctacaagttctgttgtcagagcacactaaacattatttattattttcttgattactgagttcttattttgctaccatgttataatttttgtgccattttataattcagcaataaccttaagcattttaaggtagttgttcctatgccaaatgatgcttgcatattctttgagttcttgtaactgatttttaagatagtttcaaaatttttatccctaggctgggtgcagcggctcatgcctgtaatcccagcactttgggaggcccaggcgggaggatcatgaggtcaggagttcaagaccagcctgaccaacatggtgaaaccccatctctactaaaaatacaaaaattagccaggcatggtggcatgcacctgtaatcccagctacttgggaggctggggcaggagaatcacttgaacctgggaggcagaggttgcagtgagctgagattacgcgactgcactccagcctgggcgacagagtgagattctgtctcaagaaaaaaaaaaaaattatccctatcatccctcagttctcttcattgttactgtatttatccttagcagccacctcctctttctggtatgctgttttcagtgtatgatggcatagcatagattggtcaagcttgctagagttatgcgtaaacaaagaatgaataggctctaaatggagcctgagagttttatttccagaataaagtatacataataactgttttggcttttcagaaggcatttcccttttatgtcacatgattgaagtttctggaaagctcacttatgtaaaattgggagttagaattttaatagggaaaaatcatttcatgggtaatttaaggcttactataggttgagtattccttatcctaaatgcttgggaccagaagtgttgcagatttcagatgggtttttttttttttttggattttgggatatttgcattataccattggagcatgccaaatctgagaatctgaaatctgaaatgctctggttggcatttcctttgagcatcacctcagtgctcaaaaagctttgggttttggagcatattaggatgccagattttacaccacattgcttttctttcagggctgttgatgtgtgggccattggttgtctggtaactgaaatgttcatgggggaacccctatttcctggagattctgatattgatcagctatatcatattatgatgtgtttaggtaaggttacacatctgcattttaaattttagagatttttctgcatatttgtttgtgcctgtgtgtttgtgattttttaagctgttgcattaaagattatatatgttttaagtgaaggtagggagcatataggaaaactctgtgtgtgtgtgtgtgtgtgtgtgttttgagatggagtttcgctctccttgcccaggctagagtgcaatggcgcaatctcggctcactgcaacctctgcctcctgggttcaagcgattctcctgcctcagcctcccgagtagctgggattacaggcatgtgccaccatgcccagctaatttcgtatttttagtagagatggcgtttctccatgttggtcagtctggactcgaactcctgacttcaggtgatctgcccaccttggcctcccaaagtgctaggattacaggcgtgagccaccgcgcccagccacgtgtgtgttttaatttattttttccttcagtctggcattggtctatctgatgcatgttcttttttattttttgagacggagtctcgttctgtcaccaggctggggtgcagtggcaatcttggctcactgcaaactccgactcccaggttcaagcgattctcctgcctcggcctcccaaaatgctgggattgcaggcgtgagccactgtggcccgccctggtgcatgttcttaatcacgctcacatcccctttcccctcaagagagaggaaaagagcagaggttgtgcatttctctgtacttttacataccgttaattacatgtagagaggatgcaatgatcatttattattcttgagtcagtaagcatttggtgtaatgtgactactgtccttactttcacattcaagaggagagacacagagatttccaaggttgatttaatcttggcacagatttggacttctgctattagtgcttttgacagaatttcaaagctggactatcaaaatcttacgtttgttgaaatatcaacttcatgtttgaagatggagcttagaaatggaaataatgggaaaatacttgggggtaatttaactatttgatttgaagttttcagattagaaattaaataagcagtacaatagtttcttacagtggaattttcagatatattctacattcaactagatattaaagttgagaaaaaagattaaagtgctcaaatctgactttaagataagcacaaaatcagttgaccaaatattaaattcttaaaggggagaactgatttatctagattagatcctgtccatatgactattaatatatttgttgttttattcagcactttatgctttctagttagaaacgaagtgctggtacttatatctggaaagttataaaatattttttacctgtccttttcctatgctccttaagactgccatataccaccacagtaacggcattgtctttggtcaattttgcatgttgagagagccccaaactccataagctgtgttccactatttcctctacttcacaccacctgtctcttcaccatttctatcatgttgtataataacagagatatgtgctgttttcacttccaggtaatctaattccaaggcatcaggagctttttaataaaaatcctgtgtttgctggagtaaggttgcctgaaatcaaggaaagagaacctcttgaaagacgctatcctaagctctctgaagtggtgatagatttagcaaaggtaatcatacttattgtttacactttatgacagggaatttatgcactatcaagaccttttttttttttttttacttgtctagttgatcttttttttttttttttgatggagtcttgctctgtcacacaggctggagtgctgtggcatgatctcggctcactgcaagcttcaccattcttccgcctcaacctctctagtagctgggactacaggcgctcgccaccatgcctggctaattttttgtatttttagtagaaacggggtttcactgtgttagccaggatggtctcaatctcctgacctcgtgatccgcccgcctcggcctcccaaagtgctgggattataggtgtgagccaccagccctggcctctagttgatcttttaggtcaacaatttcctctaataattttaatttttgtacatttagaaacaatttaaataatttgatgggggtaacttcagagaaaaaggactgaaaattttacctgttttaacactcattaagaaaccatttattggccgggcgcggtggctcacgcctgtaatcccagcactttgggaggccgaggcgggtggatcatgaggtcaggagatcgagaccatcctggctaacaaggtgaaaccccgtctctactaaaaaatacaaaaaattagccgggcgcggtggcgggcgcctgtagtcccagctactggggaggctgaggcaggagaatggcgtgaacccgggaagcggagcttgcagtgagccgagattgtgccactgcagtccgcagtccggcctgggcgacagagcgagactccgtctcaaaaaaaaaaaaaaaaaaaaaaagaaaccatttattgttatctagcattttgtaaatgtgaaaacatcctcactgaaaaacacattaaaaacataaaaacactgtatgatttttacgtttaacatttaaaatacacgccttgtttaaatggaaaagaaaataaaagtaggataagaaaataaaaagggtctgggcatggtggctcacacctgtaatcccagcactttgggaggctaaggccggtggatcacttgaagtcaggagttggagaccagcctggccaacatggcaaaaccccatctctattagaaatacaaaaaaattagctgggcatggtggtgcatgtctgtaatcccagctactcgggatgctgagacaggagaatcacttgaacccaggagacagaggttacagtgagctgagatcgcaccactgcactccatcctgggtgacagagtgaaactctctctttaaataaataaataaataaaggatagagaagtaggaaaatattaaaatgtagggcataggatggccgggcatggtggctcatgcctgtaatcccagcagtttgggcggccgaggcacgtggatcatgaggtcaggagttcaagaccatcctggccaagatggtgaaaccccatctctactaaaaatacaaaaattagctgggcgtggtggcacgtgcctgtaacccccgctactcgggaaggtgaggcagagaattgcttgaacctgggaggtggaggtttcagtgagctgagattacgccactgcactccagcctgggcaacagagcgagactccatgtcaaaaaaaacaaacaaacaacaacaacaaaaaaaaaacaaaatgtagggcataggatcatgatctgtatgatattgtcaaaggggtttttgcttagccactgtattggctgcagccagtttacaaatggccaatacttttttgttgtatttgattaaggaagatgttaggttttcattctatcaagtgagaatcaactctaaaatttgacctttttggggaatttttagctcaaaaagttctcatcagttaagattgtttaaatattgataagataggtacctctaaaacactaggctataagatttacccaatttttttgttttttaattctttaactcatgacgtttaataagcttataactaattaaacaagttcttagaataatacatttccagttttttacataatcttctaacagatgcaaatatttttaattggtacaagctttgtgatctatctgtaaggagatataaagggaataaaggtaaaactattgtccaacttgtgtaccttaatcataccttaaggaatcatataaaacttttaaaacattttattatggaaaatttcaaacggaaaagaaaatagaaagaatagttgtttaacaaaccttcatgtacttatcatctagtttccacaattatcaaccttttgataatcttgtttaatttatctacactctttaactttttttggagtattttgaagcaaatcccagatgtattattttacttataagtagcacagttatgcagagaaattttcttgttcatttaaagctacttattgagtcccttctatttttactggcatgttaggggttttaatgaatcagaacaagactttgcaataaaataaaataaaataaaataatctttgtaaagcatctagcatagtgataagcacaggataaatcctttcctttccattctgtaatagccttaagttttaaaaattccaaataatttcttactatcataaaatatctaattgatgtttaaagtttcattaattttcacaaaatttatttcatttcatagtttgaattaggatctaaatgaagactatagattgcaattggtcgatatatttttaaaactttatttataagcttctcttcatctctttcttttttgtttaaatcttgcaatttatttgttgaagaaactaagttgtttgtccttagaattttctacataatgcagtatacatgttcttctgtgtcctgtatttcttataaactcacagtagagctagagctcgatcagaatcaggttgattttttggcaaaactacttcataggaggtttgtgtatttccatatagagacactgaatgtttggtggtctttttgtgatactagcaaccactgacggtcattgtctagatttgtgctttattctttatccttgattattgatgtgtaaaattcattggcgggcaagtgtggtggcttgcgctggtaatcccagcattttgggaagctgaggagggtggatagcttgagctcagagttcgagaccagcctgggcaacatggcgaaacgctgtctctacaaaaaatacaaaaattagctgggtgtggtggtgtgcacctgtggtcccagctactcaggagactgagatggaaggatcacctgagcctagagaggtcaaggctgcggttaagtagtgatcacaccactgcactctagcctgggcaacagagtgagaccctgtcttaaaaaaaaaaatcgttgactataaagaggattttttttctatatttataaatttaaatgtaggtagtttttcttgatatgaactctaagtgttcttaatttttcagaaatgcttacatattgaccccgacaaaagacccttctgtgctgagctcctacaccatgatttctttcaaatggatggatttgctgagaggtatagtacttacccatgtttttcaggccataccaacctagggttaaagtcttccacaatagtcacaaatagcacttctcttatttctgttcccacctactttgcaggatctcagatatctaccactctacccccatattcccttagcaataatgcagttcctaggggtggtctcctacatttactaaattagtgctcttggttttcatgaagcggtcattttatgtcttctttctgtctccctgtggtgacgtctaccagtcagatcattgatttctatgttgctaccaactgtgtgatgccatcatcttagatgctgatgtagttacaaaagtgccatctcctattgctgccaaactcataccacaccaagtcctttgatactgctgacactgctgatgtgttggagaccttgatatgccaaacctgtgggtgccagagttgctggcatggtcttctgtgctgctcaagctgctggcaacagtgcagctgacgaaacaaagaatagtgagatgagtggcagtaacacgtagcttaataaaaggtagctaggttttcctatttgcttttgtactcaattagttgtgatatgttgtttttgtggaagtatatgaagaaactctggcctcacgtaagtagataattgtgggtattctttgattctataccaaaactctgataccatagcaaaacttgacagtggtagtttcttttttttttttttttttttttgctcttgttgcccaggctggagtgcagtggtgcaatcttggctcactgcaacctctacctcctgggttcaagcgattctcttgcctcagcctcccgagtagctgggattacaggcatgtgccaccacaactggctaattttatatttttagtagagatggggtttctctgtgttggtcaggctggtcttgaactcctgacctcaggtgatctgcccacctgggcctcctaaagtgctgggattacaggcatgagccaccgttcctggcccagtggtagtttcttaaaggctggttgcattgtggaatgtgaaaccatatcattgagtctttcttcctttgttaaattaaaatccatttgtctgtcttgcctttttaatggatcttttacccatgcatgcttttgtaatatcatgctttagtcatttggaaaatattagtttgctgagttaagcagatcttaaaaatattgatatatttcttgtacagcatcaaaaattacattctatctcataaaagttttgtgagttttgggaagctgctaagctcacagtggtggatacaagtttaacagaattgtaatttttgcataaaattttgaattttatcattaacacaattatttctgttttccttgaagtgacaagatcactttgtttatttgttaaaagaaaatgtttcccatagacccaaagtcttaataaccatagtttttctgtcagtctttctttcaagtaaaaatatctagtttagtttgtatcacaaataattatgcaagcatttttcttaagacaaccatagtgtttcggtttgtagcagaggtgctttacgtatattctcattttgtcacacagaccataaaaaaaggatgcgtttaagagtttggctttaatgaagtcaatacctttttgctgattcatcagagacattcttaagcaagactgccttttttttttttaaaaaaaaaaagcaactgagaatgtgtggttatcaagaaagtaatgactgctagtacattttggttttgctgcttttcttcttggtaaggtgtcagcagttttatccaccactgctttcatgttaacctggtgagaaagacaaacatctttatgttaaaataatagcattgactttgtgaaccttctgaatgttcttagggatccccaaagatctacacaccaccctttccaaactgctgatctaggccaggatttttctcatttttaataacatagactagtttaacattcaatttttaaaaaataaatatcttgaaagtcaatttttttgtaggtttatccatgtcttacaattttattttattatatttttatgtaggttttcccaagaactacagttaaaagtacagaaagatgccagaaatgtttctttatctaaaaaatcccaaaacagaaagaaggaaaaagaaaaagatgattccttagttgaagaaagaaaaacacttgtggtacaggtaaatgttcatgtttcacattttagatttaatatacttatttttttaaattttttgagaaaaaatatgattttgaaaaaagttttctgagcagtgagcaaattgttaacttttagtttagttgtaaaaatgtatcttctcagtagatattacctgtttagaaaacttaacatatcatgttttgagtacctgttatgtgccagactgtgcaacagtgcttaaatcacattcatccaagaacatctagatatgtgttttcatgaacacatatacacacagacacatggaacaaacaaccctcaccccacttatctttcttaaaggatatgagtgaaaaatattgtctgtagaaatttatatgaagtaaaaatttgaaaagtgagatttggatacttaacttaggtttgcttttcctggtgaactacccaggatctgttcttgataaggtaattcaaatccaccaccaatccacttatatcaagtacaccaaaagaagaaagagttatcagttttgtgttccatgactctgaatatgtaagaacaaaagtggaattcttgattcctcttccatgagtttttccactgatgtgtgtgtatgtgtgtgtgtgtgtgtctgagtgagctgtgtgaattgcatttccattctttttgtattttcccaataactatctgaataagaatctggctttaattttaatcccctaggaagaataaagaaaacagtttcaaaaaggtgatacttatgatattctgctctagttgacaagtactttataagaagaggtaaattttcattctcttcagatcattaaccttattaatatttttgttaatgatcaggataccaatgctgatcccaaaattaaggattataaactatttaaaataaaaggctcaaaaattgatggagaaaaagctgaaaaaggcaatagagcttcaaatgccagctgtctccatgacagtaggacaagccacaacaaaatagtgccttcaacaagcctcaaagactgcagcaatgtcagcgtggaccacacaaggaatccaagcgtggcaattcccccacttacacacaatctttctgcagttgctcccagcattaattctggaatggggactgagactataccaattcagggttacaggtatgaattagtaaaataagatcaaaaaagtgtttaaggacatatttgcaaattgcaagggtttgtcaattcatccacattgtttttattctcagaattctggatgctattcattcttgtcaggtagctgtttcaaaagactttttggctgtgtgatgaagattattagcatttgatcaaaacaattcaccaaagtcctgagaaagtagggttttattgcttaaggtgaagagaatctacagctatatagtctaataatgtgaaagcaggttgtcatactaaaagagataatttttgagtggctgctgctgcatggtgtgactgtgagcctatagtagtataaggaagctccatttgggaattttgctgaaaggagggtaatgtatttggtaggagttgggggctgctgggaggcatgagggtagtgttaaagaatagaatgtttcaccagatttatgtgcattgtatatttgagaatagacattacactctggttaaactgcttgttagttggtgctgtgtcatttagttttgtgcttgaaaaaataattattgctggttctaacttaaaaacatttttttttgtcgttacagagtggatgagaaaactaagaagtgttctattccatttgttaaaccgaacagacattccccatcaggcatttataacattaatgtgaccacattagtaagtaagcagttctcagtagtagaagagcaaacaccagtcagctctagacttttattaaaattaaatatgatagtgaagtattgagaattttgtttggatacacataaagtgaaaccttaattgtgcatatattatcccaacccatcatagtcataaacgtgaaattctgaaagtttaagttttatgagtctaaaaactccttccttcctttcttccttccttccttccttccttccttccttccttccttccttccttccttccttctttccttccttccttccttcctctctttctttttcttttcttttttttttcatggagtcttgctctgttgcccaggctacagtgcagtggtgagatcttggctcactgtaacctccacctcctgggttcaagtgattctcctgcctcagcctcctgagtagctgggattacaggtgcctgccaccgcacccagctaattttttatttttagtagagacagggtttcaccatcttggccaggctggtcttgaactcctgacctcgtgatccacctgccttggcctcccaaagtgctgggattacaggtgtgagccatctgtgcctggcttgttttattttttctttagtaacatagtaaattttctcaacttgatgattgctggtcagttcttttttagagtagatgaaaagataaattgtcttatctcacccacaccagatttttggtggctgtattctattggccagggacagtagcacacatctataatcccagcactttgggaggccgaggtgggaggatagtttgagcccaggagtttgagacaagcttgagcaacatagcaagatcctgtctctacaaaaaattttttaaaaagttagctgggtatggtggcacatgactgtagtcctagctacttgggaggctaagatgggtgggttgcttgagcctggaaagttgaggctacagtggtaagctgtggctgtaattgtgccccagcacctcagcctgggtgacagaccaagaccctgtctcaaaaaaaaaaaaaaaagaagaaagaaacgattctatttatttatataataaatatttatcaatcgttatgtgtgagacactggaaataaataagtcaatctttcttttcaaggacttcatgatctagaggatcagcaaattaaagcatagtatcatattgtatttagacagagcacaggaaggaccattattattctcagcatggcctccagaatgtttctcttttgaagttaggtgaaaccctcatcattcttctgctaaacttcctccattagcttcttatcactgaggaaaggccaaagtccttatagggtctataagtgcctgcaggattgactccaaccaaccctctatcccatctccacctactctttctccatcaatctgttctaccacactgatttccttgttattcctggaacatagtcagacatactcccacctttgggcttttgcacttgctatttcttttgctttggaacattcttcccccagataacccaatggatttgctttttctcttcttagctcacatgaaaccttactgtgaagtcttgtaactcattatgcctccctttatgttcgatgccttctccttgcttatttttttttccattgcatttacctgacatcgtctatatactcttatttatatattgtctgtctcccccagctagaacgtaagattccaaggcagagatttgtgcttttgttttcttcactgtacttttccttcctatgcaagtagaaaaaataattttttttttttttcgagtcagaatctcgctctgtcgcccaggctggagtgcagtggcatgatctcggctcactgcaacctccgcctcccagatttgagcgattctcctgcctcaacctcccgagtagctgggattacaggcacctgccaccacacccagctaatttttgtatttttagtagaggcggggtttctccatgtcggccaggctggtttcgaactcctgacctcaggtgatctgcccaccttggcctcccaaaatgctgggattacaggcgtgagccaccagggctggcctaaaatttaaaaatgagttcaaactaccagattaatatattcaaagagtgaaaataaaacacaaaataaatatttataataatacatttattcacaccttagaatgggggtataaaacatgggagagaggtattgtcctttaacctacaagttgaagaaagtctaggcttatctctgcttgcattaattttcattaaggaaagactccatactgaatgaatgaatgcttttcacaacaaagggaggtatggatgtattcccaaaggaatattccttgtgtgtgtgtgtttgtgtgtgtgtgagacaaatttccctctctatttctctcactatcttcagagctgaaaaatacagccttttcatctctctcatgatttggacattgtatttgtttttcttttaattattcttagtttataatttctgtgccaatagtactttcttttttggtttctttgtggatatttgtgttttaaaatcctgatttgctttggaagattattttaatcaggaaggcttttcaaaatagctggttcaataccattttgccagaactgatatcaacctgctttcaggtattgcataatcaatgctggctgataattttagatctgatttattcttctttggaagaggtaatttattcttctttggaagaggtaatcaagagaggaggtgaagattaggagggtgaagacccagtggagtctttccttaacgaaaattaatgcaagcagagctaagcctagactttcttcaacttgtatgttaaagatcaatacctctctcccatgctttatgcccccattctgatgtgtgaataaatatattatattatatattatatatgttttattttcactctttgaatatgttaatctggtagtttaaactcatttttaaattttattttttgtacttgcatatagttttctttaactattttattttataaccaacttgaaccaatagccttagcgcttgttctatgaaaagagtaagcagaaacatccaaaagaaagaaactcaaaagctcactgcaggactcttgaagactttctggtagccagaatggcataattcacagaatattaagtttgaggcatcaaggaacctccataacttaatgtcattatacatatgtttgtgtgtatgtagctcattatacatacatatgaatacaggctttctgtgattctcgaatgaaaaatattggaaaagtgtcaaattcataaggatgcagaattgaagtgctgctgctattcctcactattttttctgactttatcaactgtgaaataatagtactaagaaagatcaggaaaataattgaagttataattctttctgtgtcttaataaattgtattcctgggaagatagtattatattctaactctgaggagcaagacaaggacatatatagaaaaatgaacaacaaaattatattgggaaagtgaaaaataagctagggggagatatgatatacatgtgaagataaactgttcaaaatttatgaagacaacagcaactatttgctgaaaagcctaggaagtgtaaggcccgaccttgctggcctgaagaaccagcgcatcaatgcccatcttttaaccttttactacttgctgggacttacacactgataattggcaaaggaagaaggagaaactcatagcactcaagttatctacttgttagcaattttaatgaatttaatgaggaagacatttgaaataattggatgaattttttttgtattattaatgtccttcaaattttttattatagaccttcaatttcacaaattttaggatatgaacataggagttacagaagaaggaaagcctagctaaccttgaaactaattcttttttgttgactatgttgatgatattaggtctctagtgaaaaaaacctcttttgggcaagtaagaaaagaagagaatactccaggacagatgtccgtttgcctgaactaaactataatcatctccctgaactaagagcactgggaggcataggtgtgttgataatgcttttactttgtgtagtctgtttagattatttaaaatgtatgttagacagaatttaaaaataaaaattcatactaactttacatatttaagtatgtaaatacctgttataataagtacatactgaggaacacgtagagaatgtgaaaaactaaaaactaacaaaatgtctaatattttcactgtgtttatggtcatttctatctagctcgaaattccaggctaacaaagaaagagagcaaaattctttcagaatctcgaattccttctctggctgctattgacctgcacacccccagtattacattacatcaggtacagaaatactctttataggatggacagaactcgggtctctctcttacttactagtaaagtattttagggagagtttgctcttaagaattgtgcaggtttattggtgggatctataacattttgcttgcaaatgttgtttgattcattgcacttagtacctttattacatatatatttacataaacatttcctaacttcccataatactttctttttttagagacatggtctcactctggtgcccaggctggagtgcagtaacatgatcccagctcactgcagcttcaaacccttgagcttaagcaatccaactacctcagcctcccaagtagctgagattacaggcatgcaccaccatatctaattaatttttaaattttttgtagagatgaggccttgctatgttgcctgggctggtctcaaaactcctggcctcaagcgatccttctgctttagcttcccaaagtgttggaattacagatgtgagctatggctcctggccagtaatccattttttataggttaaaaatgtgtgtgtctgggtatgcagcctagctacagtttttgttagctactggggttttttttatatttcgtagtatttcactacattaactccaaagtaataaacaacctacctttcaaagggagaaactctggaattgtttttattcttccagaatagattatctatttgacatattttcaccaagacaatccattttatccatgttaaagatttgctattccatatagtcatttttttctatcattgctcttaacttttcagaatatatgtgtttctattacctttatatcaacagccatatcactcatttttacactgaaaaaactggcaaacttaaatctccttagccaaccataactagctacaaactttggctatggacattgtactttccttttactgcttcagacagatttttcacctttgcttgaagctaggcaacttttgttttagaattaattctccatcctcattattaacacatttccatttcatagagatttcctgtatgttgttctttatccacggtgatgctttttccactgttaactctttttttatttttattttttgagacagagtctcactctgtcgcccaggctggagtgcagtggcacaatctcagctcactgcaacctttgcctcccaggttcaagtgattcttgtgcctcagcctcccaatcagctgggattacaggtgcatgccaccacacccagctaatttttgtatttttattagaggcagggtttcaccatgttggccaggctggtcttgaactcctgacctcaagtgatctgcctgcctcagcctcctgaagtgctgggattataggcatgaggcattgcccccagcctcactgttaactcttcaatagttctgtagagctctacaaaggagtagtgttactgtgaggataacaaattatctagagatgctgatgaaagggtttattgccgtgattgctttgccttatctcttgccaacaatttctatagcaacatttaattaaaactgaacaaattcctgtcatgtgaagagataatcactgtcacagattccacatttaggaaccatagagtatcaatacatttcttatatacttctttacttcttttttttgagacagggttttgctgtgtcatccaggctggagtgcagtggcaacatcatggctcactacagccttgatcccctgggcttaggcaatcttcccacctcagcttcctgagtagctgggaccacgggagtgcaccaacacacctggctatgagccactgtacctggcctctttttttttttttttggacacatggtcttgctctgttgcccaggctggagtgcagtggcatgctcatagctcactgcagccttgaactcctgggcttaagccatctttctacctcagtctcccaaggagctgggactacaggtgcaggctaccatgcccagctaacacaattttttttttttttagagatggagtctatttatctgtttatccattttccagctgagggacagttgagttgtttgcagtttttgacaattaaacgtgaagtgttatataaacatttgtaaatgtgttttttgtgtgtgaatatacagtttcacatttagttggtaaatacctgaaaatggatttgctgggtcacttggtaagtatatgtttataagaaactatcggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcaggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaaaaaagaaactatcaaatttttccaaaatgactacactattttgtatttcaaccagcaatgcataataatttcagttgtgccttatcctcaccaatacttggtattgtcaatttaattttagcctttctaataggtgtgaagtggcatctcattgtggttttaatttgtatttccctaacgacaaagatgttgagaatcttttcatgtgcttatttgcacatgagagagctgtatatcttctttgatgaaatatctgctcaaatcttttgcccatttttaatggttgtttgttttcacattgttgagtcattatttattttgatgttcaaattagtccagtttttctagtgggatccccttcaaggtagcttctgtctccttttgacaagtcctcatcattctttgaggaattcctttgaggaattcctttgaggaattccttgctatctggcacaacagataatgcagatggatttgtaccagccagaaatcagccatctctctaaggatccctaatttcctttagtagaaaatcatatttagaaattaagatctgggctctaggtgtgtccactgatttggggggagtcattgttccaaggccctcttagtggacagagctaggtacatagacatatgtatatacatacacatttactgtgagtttatttctatttctacctatggttactgaaatctacaaagatacctctaattgtgaatccaacacccaggtttggcctagtttactattctcttctttaacagtaagaaatttagctgtatgttattccatgtataatttaatatttgtacttatttgattaattattttatatatatctaaactcctattactgctactatgatttcccttttttaaaaaagatatatttgtcatgttttaacattaaggttttgttagcttcataaaacaagttgagcagtggtctctctttatttcctgaaaaagtttaataagttggtgttatttgttccttgaatattaatggaattcactggtgaagccatctgtctcaagagctttattctggggaaagttttggataattttttttttttttttttgagactgagttttgctcttgttgcccaggctggagtgcaatggtgcgatctcggctcacggcaacctccgcctcctgggttcaagtgattctcctgcctcagcctcctgagtagctgggattacaggcgtgcaccaccacgcccggctaattttgtatttttagtagagaaggtgtttctccatgttggtcaggctggtcttgaactcccgacctcaggtgatccgcctgccttggcctcccaaagtgctgggattacaggtgtgagccaccgcgcccagcctgatttcagtttcttttttttttttttttgagacagaatcttgatctgtcgcccaggctggagtgcaatggctcgatctcggctcactgcaagctctgcctcccaggttcatgccatcctcctgcctcagcctcccaagtagctgggactacaggcgcccgccactatgcccggctaatttttttgtatttttagtagagatggggtttcaccgtgttagccaggatggtcttgatctcctgatctcgtgatccgcccgtctcggcctcccaaagtgctgggagtgtgaaccactgcgcccggcctctcaatttctttaaaatatacagtgctattcagattttctgtttcttcttgtgtctcttttggtaggttgtattttttaatccttttgttcgtgtcatacaatttttaaaatttgttggcatgacgttgttcatagcattctcttcttgtccttttgttttgtttttgcttttgagacggagtcttgctctgtcacccagactggagtacagtggcacaatctcaactcgctgcaacctctacctcctgggattaagcgattctcctgcctcagcctcccgagttactgggattacaggcatgagccactacactcggctaatttttgtatttttagtagagacggggtttcaccatgttggccaggctggtctcaaacttctaacctccagtgatacacccacctcagcctcccgaagtgctgggattacaggcgtgagccactgcacccggcctcttattgtccttttaatatctgtaaggactgtgatgattattccttatttatttttactttattatgttcactttctttttcttgatcagtatagttaggagtttgtcaattttgttgtgtttttcaacaaaatcaagcctttggcattgttaatttcctctgttatctgtcagttccctattttattgatgtctcctcttagttttccccttttacttactttgagtttattctctcaatttttctaggttcttaaggaaacccatcacttggtcacttagttttttgttttttgttttgttttgttttgtaatgtaagcatgtaaagctacacattttcctctaagcactgctttaccttcatcccacagcttttgatatgttgtattttcatttctatgcttactgaaatctacaaagatacctttaattctaatccaacatcacagagtttggtgtagtttgctatcctcttctttgagagtgagaaaattagctgtatgttattctatgtataatttaatatatttacttatttgattaattattttgtatatatctaacttcctattactgctactataatttcctttttttaaaaaagatatctttgtcatgttttaacattaaggctagtccaggctgggcgcagtggctcacgcctgtaatcccagcactttgggaggctgaggtgggcagatcacaaggtcaggagatcgagaccttactggctagctaacatggtgaaaccccatctctactaaaaatacaaaaaaattagctgggcgtggtggcaggcgcctgtagtcccagctactcgggaggctgaggcaggagattgatgtgaacctgggaggcagagcttgcagtgagccaagatggtgccactgcactccagcctgggtgacagatcaagactccatcttaaaaaaaaaaaaaaaagaaaggttagtccaaacgttttctaatttcccttgtaatttcttctctcacctatgaattatttagaaatgttgtttaatagccaggtatttgaggtttccctagatatcttattactgattaatttgtaatttaattccattgtagtcagagaatacactcagtgtgatcttgatttttttagattcaatgagacatgttttatggtctacggtctatcttggtgattgacttcattcccttgaaaaaatgtatattctttgttgttggattaattgttctataaatgtgaaattagatcaaggtggttaatagtacagtattcttcaggtcatctatgctcttaattttttttgtttaggtctatcagttcttgaaagagtgctgttaaaatcttttattatgttgtagaattgcctatttctctcttatatctgtttgttttaatttaatttaattaattaatttatttcaagacagagtctcgctttgttgcccacgctggtcttgacctcctgaactcaagtgatccacccacatcggcctcccaaagtgctgggattacaggcatgagccaccatacctggcctgtatctatttatttttgcttcatgtattttattattatttttttagaaatggggtctcactctgttgcccaggctggagtgcagcggcatgatcatagctcactgcaaccttgaattcctaggatcaagcgatcttcccccatcaacctcctgagtaactaagactacaggcacagctcaccacacccagctaattttttcttttttgttttgagagatggggtcttgctctgatgttgtggctggccttgctgtgttgctcaaactcctgacccaaactcctgacctcctcctgagcctcttgagtagctgggattacaggagtgagccaccatgcctggctgctttatgtattttaaatctctattattaggtatttgcacataattgtcttgtctttctgatacattgtaccttttctcattatgaaatgcctctctttatctctgtagtggtctgtgctttgaagtctattttatctgatattaaaatagccattccaaacttcttatacgtatgtttgcatagtaaatcttgttgatttcatttactttcaccctatctgttttttatatttatatttaaagtgtttctttcacagacagcatataaataggatttttcctttgtatacattctgtcagtctttgccttttaattggaatgtgtattcgtttattatttctgctaccttagagatgtggtagtccccccttatctatagttttgctttctgcactttcagttacctatggtcaactgtggtctgaaaatgttagtggaaagttccagaaataaacagttcttaagttttaaattgcacaccattttgaatagcctgatgaaatcttgagctgtctggctctgttctgctcaggacatgaattgtcccattgtcccgtgtatccatgtatgctacctacccttttattgcttagtagctgtctaggctttcagattaaaaaaaacacaatactgtatatttagggcttggtactatctgaggtttcaggcatccgctgggggccttggaacatatccctcatggatcggggggactacttaacacactaaattaatatctatttattttctcacagtttctgggtcaggagctagggcacagctgcttctctgaatgagtctcacaggtggaaatcaaggtgtctgcagggctgcattattttctggaggcattggggagaaaccatttccaggttcacttgggttgttgggctaatttagttccatatggttgtagggcagaagtccctatttccttgctggctaacagctgggggttgttctcaccttctagatgccactcacattccttggctcatggcccacttcctctgccttcaaaaccagcaacagtggtcaaatctctcttacacttgaatctctctgaccactccttctgccacatctctgacttactctgatgctaagagctcacatgattacactcggcccacgaagataatctatcctaattagtaacatttaacagctaattagtaacaattaacagctaattagtaacatttattccatgcacaaagtcccttcatatcggtactgaaattagtttgattaactagctagggacaggaatcttaagggcatagctttacaatcctgctgatcacagaatgtttagaccattaacatttaatgtgataattgatatttttgaatctagtcccatctttttattatttgttttctgtttgctcctctgtttttcctttccagttttcttttgtttttgaatagtttcagtattttttcggcaatatctttttatattttttttagtgattgctcgaggaattacaatatacatcactaatttttcgtagtctacattgtaccccttgaaataaaaagtacgaattgcccatccatttctccatcatccaggaaagacgttggttttgaggttgacatacctataaaggacagtaactaccatggcttccaccgctttggcaaaacctcagatgtgtggtcttctggccaaatgtctgcaatttcatattgttggagcctttattgtatccctgggggttgcagctgtctgtaagattgctgtggctgaaccaagaaagaagacatatgcagatttctacagaaattatgattccgtgaaagatttggaggagatggggaaggctggtatctttcagaatacaaagtgattttggaatgcaaaggatttctttgggttgaattacctagaagtttgtcacttacctgtgttcctgagctatgaaacatgaatatgtggtctaagaaatagtttctcttgataaataattaacaacaaaaaaagtaggaacattgctaacatattcccttcttcccagtttgcactttaagttagaacttcatatgtactacatttacatatctccagaaccccaatacgttatatgttttgttttaaacagtcataaatagtttaaacaatttatgagaaaaagttgtcttttatatttacctagctctttaccatttctgtcggtctctcttcttccctagattttcctctggtattattttccttcagcctgaaaatcttcttgttagcatttcttgcagagcaggtctgacggtgacaaatgttcttagtttttcttcatttgagaatgactttattttactttaattcttgaagtatattttattggatatggaatttgggttgacatatttttgtttttgtttttcctcccagcactataaagatgggattcttctgtcttctgatctctgtaatttctggtgggaaattctcaggcatttgaattgtttccctatatgtaatgtttcattttcctctagctgctttcaagatttaatttttatcttttcttttcattagcttaaatatgatgtatctagacatagttttctttgagtctgtcctgtttggggtttgccagccttcttgaatctttaaacttatatattttaccagatttggggagttgttggtcatgttttttgaaatttttttttctgccataatctctcacctctccttctgagagtctaatgatatgtatggtagaccatttagtattttctcataggttcccgagactctgttcactttttcttaatcatttttctttctgttcttcagattgggccacttctattcatctatcctcaagttctctgactttttactttatcatcttcattctgctattaagtccatccagtgagattttaaaatttcagatactgtgctttttagtcccaaaatatacatttggttcccttttatagcatctgtttctctgctaagaattcctatcccttcattcatttcaagtatgttttcctttacttcatgtagcagagttataagggctgcttcaaagtctttatatgatggttccaacaggtttctgtttttgtatttttgtaataagtagaagagatgggctgtagtgggcttacactgccttggcagatcagaagtatcttttaacatatgcataaactaagatcaaagaataacatccactcagcagtcatgtttgggactaaagaaaaggaaaagaacaatgtccccttacttcattcttttccatgtaggcaacttgagctatgaatagttaattataaacatctaatatttgaacaaatagaattgaaattttggggaagtggaacttttgcaattactgagaacctatgatgctctattattaatttataggtatcaggacctcccctgtcagatgattcaggggctgatttgcctcaaatggaacaccagcactgagaaccattttggttctgaactgggtaggtgtgatttcccatacttaaagctgtgcagcctttcacaggtactattttgtggcttgctgaataccaatttttcagctgaaaccgtctccagtgattgattccatgagaacttctgatagagaaagagttgtgccacttagaaaaccagaagtagtatttatctctgagatgaaatcccttttcgcagagcgtacatctcttgctcttaaactaatctcctgcattttattggatatagtaatgtgaatttagggtgacaagtgctcaagaaagatatatacctatctcccttatcagtcatttgccctgtgggcctgtaaagcaagattgattgattataatgatgcctcggttaatgttctgaaagatgcatccttaggtgatttcactgttatgctaacatcacagtttacttacacaaacctagatgatataacctactatactcctaggctatatggtatagcctattcctcctaggctacaagcctgcacagcatgttactgtactgaatactgtaagcaattgtaacacaatagtaaatattcatatatcaaaacatagaaaaggtaatgcattgcactatgatgttactagctacaatgtcactaggcaataagaattttcagcttcatgataatctatgggactaccattgtatatgcagctcattgttgacctaaacattgttatacagtgcatgattatacttggcaatatttttcccaataaagagagatcagtgctataccaaattattgacttccttttaaatataacttttttagtattaaaaaagccaaggctgggcgcggtggctcacgcctgtaatcccagcactttgggaggctgaggcgggcggatcacgaggtcaggagatcgagaccatcctggctaacacggtgaaaccccgtctctactaaaaatacaaaaaattagccgggtgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaagaaaaaaaaaaaaaagccaaaacaaaaatcagacattttttttgagatgggatcttactgtattgtccaggctggagtgcagtggcgtgatctcggctcactgcaatctctgcctcctaggctgaagcgatcctcccaccccagcctcctgagtagctggggccacacgtgtgtgccacaaagcccggctaattttttgtatttttggtagagatggggtttcaccatgttgcccagggtggcctcaaactcctgagctcaaatgatctgcccgtcttggcctccctaagtgctggaattagaggtgtgagccaccatgtctggccaatcagaccattttgaatgataatttatttgctcaagcaagtcattcaatgtttaatatctcattatgtaaaatattatgatgataaattcccacattctcacaactgtatttatttatttttttgagacggtgtcttgctctgtctcccaggctggagtgcagtggcacaatctcagcttactgcaacctccaagtgatcctcatgcctcagcctcctgagtagctgggatcacaggtgtgtgccaccacgcccagctaatttttgtatatttagtagagatggggtttcactatgttggctaggctggtcttgaactcctgacctcaggtgatctgcccaccttggcctcacaaagtgctgggattacagtcatgagtcaccatgcccagcccctaactgtattattggaattaatctgttaacctttacaaagcattttgagcatctgtgacatcaaaggtgaatgtactgtgaagtgaacaaagcttaagttttgatctttcatttgcacaggtcctttcaaggccgtgggagagtctttgcaatgcattcatttgagttaattcatgttgagcaatgaacaactaattcatacaaaccgtgtaaactacagggtctgcaaaacttggatgcattttgtcaaaattagtcattatttgcataaaaggagatgtgtgttacacatgatagtttttcttttttttgagacggagtttcgctcttgttgcccaggctggagtgcaatggcgtgatctctgctcactgcaacctccacctcccgggttcaagtgattctcctgcctcagcctcctgagtagctgggattacaagcatgggccaccatgccgggctaatttttttgtatttttagtagagatggggtttctccatgttggtcaggctggtcttgaactcccaacctcaggtgatctgcccgcctcggcctcccaaagtgttgggattacaggcgtgagccactgcacccagccatacgcaagatagttttgattggtaattattctaaagattgtcttaaaaatttgcttttgatacatctaattgaaatctaaggttttcttaaactacatagtacccaccaatgttatcaatatatacgatgtgagaaagcaagtgagatagaaattaatttttagaaagattttttgtaaaatagttggtggtatatcctcctgtgcttttagatgatgctcttgcacttgagatgacatcttcttgcagcaagagtgctgatatcccaagaggagagattcatggttttgatcatttccttctgaactgcctgcattttctgaggaaggccttctagaagaaggaaagacaaagacttccaaatgtttcaaaggaagattgaacaaatggccctccccaactgttatcccattacctttcacgtccaccgatgctatttcaagacatatccagtggaataacagtgatatggttcttgttacatgaatgtgtatttactgttaggagattgtatattttaagttaccatgattaaaagtgtgtaaaaaagggggacagagagaaatactataaaaggccatgttactcatgcattgaactttgtgtgttttctgtataaatgtgtacatttatttaggagtgggtttcttgggggtaggtgataaaaattaggatcagttgaagaaaattgaagaaactaggatcagtaagagaaactgtttgctttacctgaatttaactatgaaaacacacttaacaatcttacacgtttctagatattaagtgaatatgtaactcctgtccatgggtagatgtgtatctttgacttctgtaattatttttgatattctatcagtgtattatgagactctggcctccctgcagatcttctaagaaccacactaatgcaagcgtgacagagaaacctctttcgaatgacttactacaactctggcattggttagttccatgtattgtaagattctggtgctaatgctccatacagaattgatcaggactgattactcttctgtggaccaattgctattgaactacccagctgaagagggtttggggagagaacgttcattattatggactccacttttgtcccctggtagtttaagggtgatactagaatccagagaagttcctgtctccttgtggcctcaaacactcaggccgtctcaaaagcttttcaccaaggtgcaaaatcatttacctgcattccacacatcttttccaatgcatggatccaaaactttttaggtgggaggaataaactaagttggctacaggttcttgatttctgggtttctacacatgctctgcatttatcattttagagaatggttgtttagggaaccagaatttgtgagttttttttttttttttgccttaactaattaatagtgtgttgctttttttccaaagccattgactagaacttagctgaagaatttaactagaacttttctttattactctgaggagcttaatactgaaattctgaccttggatatttaagcctcaaaagagtaggacattcaagaggaggcaaataaggggtttcaatgtacaagtctataggtggtggttaaattaaaattggtcttgatttaaaagctctacttttaatctgtgaccagctgtttgacaacattagtatggataacatgctgaaaaaactagggtatcagcattaataggctgcatattaaaaagttactactttatttttactttgaaaaacactgccaatagattctgcagcatggcaaacaggtaatacttaaagccaaatggcgcttcggcttatttcaatactatttttatgtaaagatcagtgtgagaatatcagctcaatggttgaggttctaggatcaattgccttgcagtttttatactagcttttgatatcttttgcctattaaaacactttgtgaactttagaattaaggaactaaaaaatatttaccataagtccaagttatgacttgaggatcatatctgattagaactgtggcagaactactagaaaaagattctgcaaattggtttcagtgattcttgttgatgtaatcaacaagattacataatcaacaataatagatattgaaccaaaatgatcctttttttaagtttaaggttatttagtttctcttgcaaggatatgatttagtcacacactaatagggaatatgtgtatgtactggtggggagcagtgggcttgctgcatgtatgtatgttttaaaattaataaatattatcttactgggctccatagatggtttaaatttagatttatttaattaagaggctataaataatatatacactactgtatggtatttaacaaataagttttaaatctgttaattcttctggaaaactttggatgtagggatggcaagctaattttcttgtatagctggtgatataagcagcatggtcataaaataacatttcttataaagactaactggtaatagttgtgacagaacctggttttcctgactgagaatcccttgcttgcttagaatatctgacttcccattgaaatggaacatagaaaatgtgttttcttatgtgactctcattattggttcctttatcttagttgtgctacaatataaggctggacctgtaatacactatgtatcagactagactgaaaaatgtgatcgatttgttttctaagtcctcctctatataactgaaccatggtcaggggattagagaagcaacagagcactgctcctaaggctttaggctagtcttagtgaattggactgaatggatttttctctgttgttttttgcattaagtgtttttgcactagaagagggtgcttaacactcctctatctatatgaataaaaaattatgttctcacattgttttctcaagggtcttattttttctcttgacagcttcagacatgaaataactagaaaatgtaattaatgtacagcatatttttttcttaacatgccctctgagaagtaagtaccttatgttatcactcgtgggtaacttgtgatttagagctttgtgtttatagatatttgtggatatctgatgttgaatatattctgagaaattttctttaaaaccaaaacccatccacgagacagcacttttctttgttttcagtgaaaatttagttttgcccattgcacattcttaatttctatattctctactttggcactcagggccatctcctgtagaagtgaagtttctggtaacattttggaaaatgtattgtcgattaatatgaacatataagaaaggttcagggaaatgaaaacaacatatctcaattcagcatgttttcctatccctaagctagggtaggttctcctatcagtctggagaacaggctttcatatcagtttacctccatccagctatcccacctgctctagacctggaaagtgatcttcattaatcttgctgctaggatttaaaagtatcccctgaccaccccca

SEQ ID NO: 9 - RNA, 24 nt, synthetic construct
  source 1..24: mol_type = other RNA
agggtgaagcccttttggcagacg

SEQ ID NO: 10 - RNA, 24 nt, synthetic construct
  source 1..24: mol_type = other RNA
aaaccgtctgccaaaagggcttca

SEQ ID NO: 11 - RNA, 24 nt, synthetic construct
  source 1..24: mol_type = other RNA
agggagacgaggggcgagaaggtg

SEQ ID NO: 12 - RNA, 24 nt, synthetic construct
  source 1..24: mol_type = other RNA
aaaccaccttctcgcccctcgtct

SEQ ID NO: 13 - DNA, 33 bp, synthetic construct
  source 1..33: mol_type = other DNA
tcgtcggcagcgtcagatgtgtataagagacag

SEQ ID NO: 14 - DNA, 34 bp, synthetic construct
  source 1..34: mol_type = other DNA
gtctcgtgggctcggagatgtgtataagagacag

SEQ ID NO: 15 - RNA, 23 nt, synthetic construct
  source 1..23: mol_type = other RNA
cacatagcttgagagcgtgcagg

SEQ ID NO: 16 - RNA, 23 nt, synthetic construct
  source 1..23: mol_type = other RNA
acatagcttgagagcgtgcaggg

SEQ ID NO: 17 - RNA, 23 nt, synthetic construct
  source 1..23: mol_type = other RNA
acgtcaccgaccagcagttctgg

SEQ ID NO: 18 - RNA, 23 nt, synthetic construct
  source 1..23: mol_type = other RNA
aagtatggcaagtaagcggcagg

SEQ ID NO: 19 - protein, 281 aa, Homo sapiens
FEILGVVGEGAYGVVLKCRHKETHEIVAIKKFKDSEENEEVKETTLRELKMLRTLKQENIVELKEAFRRRGKLYLVFEYVEKNMLELLEEMPNGVPPEKVKSYIYQLIKAIHWCHKNDIVHRDIKPENLLISHNDVLKLCDFGFARNLSEGNNANYTEYVATRWYRSPELLLGAPYGKSVDMWSVGCILGELSDGQPLFPGESEIDQLFTIQKVLGPLPSEQMKLFYSNPRFHGLRFPAVNHPQSLERRYLGILNSVLLDLMKNLLKLDPADRYLTEQCLN

SEQ ID NO: 20 - protein, 277 aa, Homo sapiens
YTTIRQLGDGTYGSVLLGRSIESGELIAIKKMKRKFYSWEECMNLREVKSLKKLNHANVVKLKEVIRENDHLYFIFEYMKENLYQLIKERNKLFPESAIRNIMYQILQGLAFIHKHGFFHRDLKPENLLCMGPELVKIADFGLAREIRSKPPYTDYVSTRWYRAPEVLLRSTNYSSPIDVWAVGCIMAEVYTLRPLFPGASEIDTIFKICQVLGTPKKTDWPEGYQLSSAMNFRWPQCVPNNLKTLIPNASSEAVQLLRDMLQWDPKKRPTASQALR

SEQ ID NO: 21 - protein, 280 aa, Homo sapiens
YENLGLVGEGSYGMVMKCRNKDTGRIVAIKKFLESDDDKMVKKIAMREIKLLKQLRHENLVNLLEVCKKKKRWYLVFEFVDHTILDDLELFPNGLDYQVVQKYLFQIINGIGFCHSHNIIHRDIKPENILVSQSGVVKLCDFGFARTLAAPGEVYTDYVATRWYRAPELLVGDVKYGKAVDVWAIGCLVTEMFMGEPLFPGDSDIDQLYHIMMCLGNLIPRHQELFNKNPVFAGVRLPEIKEREPLERRYPKLSEVVIDLAKKCLHIDPDKRPFCAELLH

SEQ ID NO: 22 - protein, 277 aa, Homo sapiens
YTTMRQLGDGTYGSVLMGKSNESGELVAIKRMKRKFYSWDECMNLREVKSLKKLNHANVIKLKEVIRENDHLYFIFEYMKENLYQLMKDRNKLFPESVIRNIMYQILQGLAFIHKHGFFHRDMKPENLLCMGPELVKIADFGLARELRSQPPYTDYVSTRWYRAPEVLLRSSVYSSPIDVWAVGSIMAELYMLRPLFPGTSEVDEIFKICQVLGTPKKSDWPEGYQLASSMNFRFPQCVPINLKTLIPNASNEAIQLMTEMLNWDPKKRPTASQALK

SEQ ID NO: 23 - protein, 278 aa, Homo sapiens
YKAIGKIGEGTFSEVMKMQSLRDGNYYACKQMKQRFESIEQVNNLREIQALRRLNPHPNILMLHEVVFDRKSGSLALICELMDMNIYELIRGRRYPLSEKKIMHYMYQLCKSLDHIHRNGIFHRDVKPENILIKQDVLKLGDFGSCRSVYSKQPYTEYISTRWYRAPECLLTDGFYTYKMDLWSAGCVFYEIASLQPLFPGVNELDQISKIHDVIGTPAQKILTKFKQSRAMNFDFPFKKGSGIPLLTTNLSPQCLSLLHAMVAYDPDERIAAHQALQ

SEQ ID NO: 24 - protein, 280 aa, Homo sapiens
YEKIGKIGEGSYGVVFKCRNRDTGQIVAIKKFLESEDDPVIKKIALREIRMLKQLKHPNLVNLLEVFRRKRRLHLVFEYCDHTVLHELDRYQRGVPEHLVKSITWQTLQAVNFCHKHNCIHRDVKPENILITKHSVIKLCDFGFARLLTGPSDYYTDYVATRWYRSPELLVGDTQYGPPVDVWAIGCVFAELLSGVPLWPGKSDVDQLYLIRKTLGDLIPRHQQVFSTNQYFSGVKIPDPEDMEPLELKFPNISYPALGLLKGCLHMDPTQRLTCEQLLH

SEQ ID NO: 25 - protein, 279 aa, Homo sapiens
YETLGKVGEGSYGTVMKCKHKNTGQIVAIKIFYERPEQSVNKIAMREIKFLKQFHHENLVNLIEVFRQKKKIHLVFEFIDHTVLDELQHYCHGLESKRLRKYLFQILRAIDYLHSNNIIHRDIKPENILVSQSGITKLCDFGFARTLAAPGDIYTDYVATRWYRAPELVLKDTSYGKPVDIWALGCMIIEMATGNPYLPSSSDLDLLHKIVLKVGNLSPHLQNIFSKSPIFAGVVLPQVQHPKNARKKYPKLNGLLADIVHACLQIDPADRISSSDLLH

SEQ ID NO: 26 - protein, 279 aa, Homo sapiens
YEKLAKTGEGSYGVVFKCRNKTSGQVVAVKKFVESEDDPVVKKIALREIRMLKQLKHPNLVNLIEVFRRKRKMHLVFEYCDHTLLNELERNPNGVADGVIKSVLWQTLQALNFCHIHNCIHRDIKPENILITKQGIIKICDFGFAQILIPGDAYTDYVATRWYRAPELLVGDTQYGSSVDIWAIGCVFAELLTGQPLWPGKSDVDQLYLIIRTLGKLIPRHQSIFKSNGFFHGISIPEPEDMETLEEKFSDVHPVALNFMKGCLKMNPDDRLTCSQLLE